(19) United States
(12) Patent Application Publication
(10) Pub. No.: US 2018/0199611 A1
(43) Pub. Date: Jul. 19, 2018

(54) PROPIONIBACTERIUM FRUEDENREICHII AS A PROBIOTIC FOR INFANTS

(71) Applicant: UNIVERSITY OF FLORIDA RESEARCH FOUNDATION, INC., GAINESVILLE, FL (US)

(72) Inventors: MANSOUR MOHAMADZADEH, GAINESVILLE, FL (US); BIKASH SAHAY, GAINESVILLE, FL (US)

(21) Appl. No.: 15/573,502

(22) PCT Filed: May 12, 2016
(86) PCT No.: PCT/US2016/032096

§ 371 Date: Nov. 13, 2017

Related U.S. Application Data
(60) Provisional application No. 62/160,698, filed on May 13, 2015.

(51) Int. Cl. A23L 33/00; A23L 33/135; A61K 35/741; A61K 39/39

(52) U.S. Cl. CPC A23L 33/40; A23L 33/135; A61K 2035/115; A61K 39/39; A61K 35/741

(57) ABSTRACT

The invention pertains to the benefits of Propionibacterium freudenreichii supplementation in infant foods, for example, preterm infant foods such as human donor milk or infant milk formula. Accordingly, the invention provides a method of administering an infant food comprising a pharmaceutically effective amount of P. freudenreichii to an infant, for example, a preterm infant. The invention also provides infant food compositions comprising a pharmaceutically effective amount of P. freudenreichii. Further, the invention pertains to methods of determining a pharmaceutically effective amount of P. freudenreichii which provides beneficial effects to a subject, for example, induction of regulatory immunity and/or induction of protective immunity. Also provided are compositions capable of modulating the immune response of humans and non-human mammals and methods of modulating the immune response of a subject using said compositions.

### CROSS-REFERENCE TO RELATED APPLICATION

This application claims the benefit of U.S. Provisional Application Ser. No. 62/160,698, filed May 13, 2015, the disclosure of which is hereby incorporated by reference in its entirety, including all figures, tables and amino acid or nucleic acid sequences.

The Sequence Listing for this application is labeled “SeqList.txt” which was created on May 11, 2016 and is 17 KB. The entire content of the sequence listing is incorporated herein by reference in its entirety.

### BACKGROUND OF THE INVENTION

The complexity and stability of the human intestinal microbiota is established with age. A wide range of factors influences the intestinal microbiota, including feeding mode. Human breast milk is preferred for both term and preterm infants. This is not possible in all preterm infants, thus formula or pasteurized donor milk is commonly substituted. Consequently, intestinal colonization, due to the absence of microbes that these infants would be receiving from their own mothers' milk, will be significantly altered.

The nutritional components of human milk significantly influence both the developmental processes and health of newborn infants. Human milk provides both nourishment and a large amount of bioactive compounds that significantly affect the optimal regulation of local and systemic immunity, cognitive development, protection against microbes and their toxins, and perhaps most importantly, influence the establishment of the gut microbiota. This microbiota and its corresponding macro- and micronutrients play a critical role in determining the outcome of various signaling events in host cells in order to maintain healthy intestinal homeostasis. Gut bacteria, or even a single bacterial species, can significantly impact the commitment and/or maintenance of various intestinal T cell subsets, including Th17 and regulatory T cells (Tregs). Gut bacteria can also determine the regulation of protective immune responses against disease progression.

In both scenarios, innate cells, including dendritic cells (DCs) expressing pattern recognition receptors such as C-type lectins (CLECs), are the initial targets of the microbial products to sustain physiological homeostasis. Deterioration of this healthy homeostasis may result in devastating inflammatory diseases, including IBD and colon cancer, all of which are closely linked to chronic intestinal inflammation. Experimental murine models have already shown that reprogramming elicited proinflammation is protective in chronic T cell-induced colitis and colonic polyposis. Unfortunately, efficacious interventions for reshaping such intestinal inflammation to block proinflammatory factors (TNF, IL-1β) have not been forthcoming, as a large number of patients either fail to respond or experience relapse with such a therapy. Hence, deterioration of gut microbiota, their products (e.g., butyrate) and their balanced nutrients are linked to the pathology of various diseases, including colitis and colon cancer.

### BRIEF SUMMARY OF THE INVENTION

The invention provides a safe and efficient method of restoring the advantages afforded by beneficial bacteria received from a mothers' milk so that feeding preterm infants with human donor milk or infant formula milk produces the same outcome as being fed the mother's milk. As such, the invention provides a method to enhance the benefits of human donor milk or infant formula milk.

In one embodiment, the method comprises oral administration to an infant, for example, a preterm infant, an effective amount of the probiotic microorganism, for example, Propionibacterium freudenreichii, to promote development of intestinal microbiota and/or induce protective and regulatory immunity. Accordingly, the invention provides a composition comprising P. freudenreichii and an infant food, for example, human donor milk or infant formula.

The invention also provides a method to determine the effective amount of P. freudenreichii needed to supplement an infant food, wherein the effective amount of P. freudenreichii promotes development of intestinal microbiota and/or induces protective and regulatory immunity.

In addition, the invention presents a method of enhancing protective and regulatory immune responses in infants whose mothers are able to breast feed through the oral administration to the mother of an effective amount of the probiotic microorganism, for example, Propionibacterium freudenreichii.

The invention also provides a safe and efficient method of restoring the advantages afforded by beneficial bacteria received from a mothers' milk so that feeding offspring with donor milk or formula milk produces the same outcome as being fed the mother's milk. As such, the invention provides a method to enhance the benefits of donor milk or formula milk.

In one embodiment, the method comprises oral administration to an offspring, for example, a preterm non-human mammal or a full term non-human mammal, an effective amount of the probiotic microorganism, for example, Propionibacterium freudenreichii, to promote development of intestinal microbiota and/or induce protective and regulatory immunity in the nursed offspring. Accordingly, the invention provides a composition comprising P. freudenreichii and a food, for example, donor milk or formula.

In addition, the invention presents a method of enhancing protective and regulatory immune responses in offspring whose mothers are able to breast feed through the oral administration to the mother of an effective amount of the probiotic microorganism, for example, Propionibacterium freudenreichii.

### BRIEF DESCRIPTION OF THE DRAWINGS

The patent or application file contains at least one drawing executed in color. Copies of this patent or patent application publication, with color drawing(s), will be provided by the Office upon request and payment of the necessary fee.

FIGS. 1A-1B. Graphs showing differences in the microbiota composition of breast milk-fed (BMF) and formula-fed (FMF) infants. A. Reduced richness and diversity in the fecal microbiota composition of formula-fed infants. B. Among the differences observed, formula-fed infants demonstrated an absence of the genus Propionibacterium. * denotes statistical significance p<0.05.

FIGS. 2A-2B. Graphs showing the immune changes induced in germ-free (GF) mice transplanted with BMF or FMF fecal microbiota. GF mice were transplanted with BMF- or FMF-derived microbiota by oral gavage. Two weeks after fecal microbiota transplantation, mice were sacrificed and induced changes were noted. Reconstitution of GF mice with infant-derived fecal microbiota results in a reduction of pro-inflammatory cytokines in the sera, irrespective of the donor group (A); however, only BMF-transplanted GF mice demonstrated lower levels of IL-1β expression by colonic dendritic cells (DCs) (B). * denotes statistical significance p<0.05.

FIG. 3. Graphs showing the immunologic benefits of oral gavage with P. freudenreichii in the colons of antibiotic (Abx)-treated mice transplanted with formula-fed (FMF) microbiota. Conventional B6 mice were depleted of their intestinal microbiota with antibiotic treatment for four consecutive weeks. After depletion, the mice were transplanted with BMF- or FMF-derived microbiota by oral gavage and groups of FMF-transplanted mice were supplemented with 10⁹ colony-forming units (CFU) of P. freudenreichii for the specified number of times (1× or 3×). Two weeks after fecal transplantation, mice were sacrificed and immune changes induced in the colon were noted. FMF-transplanted mice showed an increase in IL-1β production by colonic DCs and a decrease in IL-10. Oral treatment with P. freudenreichii significantly reversed these DC phenotypes. * denotes statistical significance p<0.05, **p<0.01.

FIGS. 4A-4B. Graphs demonstrating the immunologic benefits in the colon of supplementing pasteurized human donor milk with P. freudenreichii. Conventional B6 mice were fed human donor milk every day for a week. One group of mice received donor milk supplemented with 10⁹ CFU of P. freudenreichii on days 0 and 3 (2×) and un-supplemented donor milk the remaining days. Two weeks after the initial gavage, mice were sacrificed and induced immune changes in the colons were noted. Donor milk-fed mice showed a decrease in pro-inflammatory IL-1β and IL-12 production by colonic DCs (A) and a decrease in pro-inflammatory IFNγ expression in CD4⁺ T cells (B). Oral treatment with P. freudenreichii significantly enhanced these anti-inflammatory responses (A and B). * denotes statistical significance p<0.05, **p<0.01, ***p<0.001.

FIGS. 5A-5B. Graphs demonstrating the immunologic benefits in the mesenteric lymph nodes (MLNs) of supplementing pasteurized human donor milk with P. freudenreichii. Conventional B6 mice were fed human donor milk every day for a week. One group of mice received donor milk supplemented with 10⁹ CFU of P. freudenreichii on days 0 and 3 (2×) and un-supplemented donor milk the remaining days. Two weeks after the initial gavage, mice were sacrificed and induced immune changes in the MLNs were noted. Donor milk-fed mice showed no differences in the frequency of FoxP3⁺ regulatory T cells (Tregs) and a slight decrease in pro-inflammatory IFNγ expression in CD4⁺ T cells (A); a slight decrease in the expression of IFNγ in CD8⁺ T cells was also noted in donor milk-fed mice. Oral treatment with P. freudenreichii promoted the induction of FoxP3⁺ Tregs and a significant decrease in the number of both CD4⁺ and CD8⁺ T cells expressing IFNγ (A and B). * denotes statistical significance p<0.05, **p<0.01.

FIGS. 6A-6B. Graphs demonstrating the immunologic benefits in the spleen of supplementing pasteurized human donor milk with P. freudenreichii. Conventional B6 mice were fed human donor milk every day for a week. One group of mice received donor milk supplemented with 10⁹ CFU of P. freudenreichii on days 0 and 3 (2×) and un-supplemented donor milk the remaining days. Two weeks after the initial gavage, mice were sacrificed and immune changes induced in the spleens were noted. Donor milk-fed mice showed no differences in the frequency of FoxP3⁺ regulatory T cells (Tregs) and a slight decrease in pro-inflammatory IFNγ expression in CD4⁺ T cells (A); a slight decrease in the expression of IFNγ in CD8⁺ T cells was also noted in donor milk-fed mice. Oral treatment with P. freudenreichii did not significantly induce splenic FoxP3⁺ Tregs, but promoted a significant decrease in the number of both CD4⁺ and CD8⁺ T cells expressing IFNγ (A and B). * denotes statistical significance p<0.05, **p<0.01.

FIGS. 7A-7B. Graphs showing that P. freudenreichii can induce both regulatory and protective immune responses in healthy hosts. Conventional FoxP3-GFP (B6) mice were either fed 10⁹ CFU of P. freudenreichii on days 0 and 3 (2×), or fed 10⁹ CFU of P. freudenreichii on days 0, 3, 6, 9 (4×). One week (2×) or two weeks (4×) after the initial gavage, mice were sacrificed and immune changes induced in the colon were analyzed. Mice gavaged four times with P. freudenreichii showed increased numbers of IL-10⁺ colonic DCs (A), and higher regulatory (FoxP3-GFP⁺) and Th17 (IL-17A⁺) immune responses in the colon (B). * denotes statistical significance p<0.05, **p<0.01.

FIG. 8. Graphs demonstrating that the benefits afforded by P. freudenreichii can be transferred through the mother's milk. Pregnant B6 mice were fed 10⁹ CFU of P. freudenreichii once a week during gestation and while breastfeeding (5-6×). Pups were weaned at 3 weeks of age, and the litters of these dams were then split into pups continuing to receive oral gavages of P. freudenreichii (10⁹ CFU) or PBS for 3 weeks. Another group of pregnant B6 were given PBS during the gestation period and their pups were divided and treated as described above. After the 3 weeks of P. freudenreichii treatment or PBS, the resulting 6-week old B6 mice were sacrificed and immune responses in the colon analyzed. P. freudenreichii promoted an expansion of colonic FoxP3⁺ Tregs in mice that were fed P. freudenreichii and in mice whose mothers were given P. freudenreichii while pregnant and breastfeeding. Similarly, Th17 responses were induced in mice that were fed P. freudenreichii and in mice whose mothers were given P. freudenreichii while pregnant and breastfeeding. However, the greatest increase in Th17 immunity was observed in mice with mothers receiving P. freudenreichii while they were in utero and while breastfeeding (last column). * denotes statistical significance p<0.05, **p<0.01.

FIGS. 9A-9B. Graph showing that protective immunity induced by P. freudenreichii is antigen-dependent. B6 WT or MHC II^{-/-} mice were fed 10⁹ CFU of P. freudenreichii or P. freudenreichii Slp⁻ on days 0, 3, 6, 9 (4×). Two weeks after the initial gavage, mice were sacrificed and induced immune responses analyzed in the colon. A. WT B6 mice gavaged with P. freudenreichii demonstrated higher Th17 immunity. These responses were not observed in MHC II^{-/-} mice. B. Mice gavaged with P. freudenreichii, but not P. freudenreichii Slp⁻, showed increased numbers of colonic Th17 (IL-17A⁺) cells. n=5 mice/group. * denotes statistical significance p<0.05, **p<0.01.

FIGS. 10A-10B. Graphs depicting the protection induced by P. freudenreichii in an infectious colitis model. B6 mice were infected with 10⁹ CFU of C. rodentium, a murine model of infectious colitis. Groups of mice were fed 10⁹ CFU of P. freudenreichii on days −1, 2, 4, and 6 (4×), with and without infection. Mice were monitored for disease onset and progression. On day 13, at the peak of disease, mice were sacrificed and immune changes induced in the colon were analyzed. Infected mice that were gavaged four times with P. freudenreichii showed better clinical outcomes as measured by decreased weight loss and significantly reduced inflammation in the colon and MLNs when compared to phosphate buffered saline (PBS)-treated infected mice (A). Protection from C. rodentium-induced colitis was associated with protective Th17 responses in the colon (B). * denotes statistical significance p<0.05, ***p<0.001.

FIGS. 11A-11D. Graphs depicting the protection afforded by P. freudenreichii in a chemically-induced model of colitis. B6 mice were given 3% dextran sulfate sodium (DSS) in the drinking water for 5 days water to induce colitis. A group of mice was fed 10⁹ CFU of P. freudenreichii on days −3, −1, 1, and 3 (4×). Mice were monitored for disease onset and progression. On day 10, mice were sacrificed and immune changes induced in the MLNs were analyzed. DSS-treated mice that were gavaged four times with P. freudenreichii showed better clinical outcomes as measured by decreased weight loss, reduced diarrhea scores and decreased fecal occult blood (FOB) (A). P. freudenreichii-treated mice also demonstrated reduced inflammation in the colons and MLNs when compared to DSS only-treated mice (B). Decreased severity of colitis was associated with reduced neutrophilic infiltration in the MLNs (C) and lower IL-1β expression in MLN neutrophils and DCs (C and D). * denotes statistical significance p<0.05, **p<0.01, ***p<0.001, ****p<0.0001.

FIG. 12. Graph depicting the binding of P. freudenreichii to C-type lectin specific intercellular adhesion molecule-3-grabbing nonintegrin-related 1 (SIGNR1). Bacteria were washed with PBS and then allowed to bind to either SIGNR1-Fc or Ctrl-Fc chimeric protein for 1 hour, before washing and incubating with Fc-specific 2° antibody for another hour. The washed bacteria were run on FACS Canto II. Data were analyzed using FlowJo software.

FIGS. 13A-13B. Graph depicting the upregulation of SIGNR1 expression induced by P. freudenreichii in vivo and in vitro. A. Mice were fed 10⁹ CFU of P. freudenreichii on days 0, 3, 6, 9 (4×). Two weeks after the initial gavage, mice were sacrificed and induced immune changes in the colon analyzed. SIGNR1 expression in colonic DCs is increased in P. freudenreichii-treated mice. B. RAW264.7 cells were either stimulated with 100 MOI of P. freudenreichii for 24 hours or left untreated. Cells were harvested, washed and stained with SIGNR1-APC conjugate antibody and analyzed by FACS. SIGNR1 expression on RAW264.7 cells is induced upon P. freudenreichii treatment.

FIG. 14. Graph depicting the binding of P. freudenreichii to a panel of lectins. Bacteria were washed with PBS and subsequently allowed to bind with the specified fluorescent-labeled lectins for 1 h. The bacteria were washed twice with FACS wash and run on FACS CantoII. Data were analyzed using FlowJo software.

FIGS. 15A-15B. P. freudenreichii used in our study was sequenced on Pacific Biosciences platform. The sequences generated were converted into one contig and compared with only known genome sequence available at NCBI on RAST platform. (A) The data show the similarity and dissimilarity between known sequence and our sequence. (B) The genome sequence was annotated on the RAST platform showing genes related to different metabolic pathways of the bacterium.

FIG. 16. Six week old C57BL/6 mice were orally gavaged with PBS, P. freudenreichii (10⁹ CFU/mouse), or the surface layer isolated from P. freundenreichii (Slp^{P. freudenreichii}) (250 μg/mouse) 4 times at 3 day intervals. Slp^{P. feudenreichii} was obtained after dissolving the surface layer proteins (Slp) in Guanidine hydrochloride (4M) for 1 hour. The solubilized surface proteins were dialysed extensively to remove salts before use in gavaging mice. Mice were sacrificed 1 week after the final gavage, and immune response in the colon was analyzed. The that mice received the Slp^{P. feudenreichii} show induction of Th17 along with a comparable increase in Tregs and a subsequent decrease in IFNγ producing T cells. This data demonstrate that the protective effects of the P. freundenreichii lie within its surface layers.

FIGS. 17A-17E. Abundance of Propionibacterium in the fecal samples of human breast milk-fed (HBMF) preterm infants. (A-E) Microbial composition of HBMF (n=20) and formula-fed (FF) preterm infants (n=20). (A) Summary box-plots of Chao Richness, Shannon diversity, and Pielous evenness indices derived from analyses of 16S ribosomal DNA from fecal samples by day±13; HBMF: blue and FF: red. (B) Phylum structure of the abundant bacteria in fecal samples by day±21. (C) Differential taxonomic cladogram of HBMF versus FF preterm infant bacterial fecal samples by day±21 (blue: HBMF-enriched taxa; red: FF-enriched taxa). (D) Percentage of OTUs of Propionibacterium genus analyzed from the fecal samples by day±13 and day±21. (E) Relative abundance of different Propionibacterium species (e.g., P. freudenreichii). The P value in A, D and E was determined by a two-tailed unpaired t-test. Error bars, S.E.M; *P<0.05, **P<0.01.

FIGS. 18A-18B. First draft genome-sequence analyses of P. UF1. (A) Genome sequence comparison of P. UF1 with known P. freudenreichii spp. Shermanii CIRM-BIA1 (red) and P. freudenreichii spp. freudenreichii DSM20271^{T} (blue). Prophage was identified by PHAST (Phage Search Tool) (green). Similarities and differences between the known sequence of P. freudenreichii strains and the sequence of P. UF1 are shown. (B) Correlation of the annotated genome sequence on the RAST platform with different metabolic pathways of P. UF1.

FIGS. 19A-19B. Abrogation of proinflammatory responses by fecal microbiota of HBMF preterm infants. (A-B) Germfree (GF) mice were transfaunated with HBMF (blue), FF (red) microbiota, FF microbiota treated four times (4×) with P. UF1 (green), or left untransfaunated (black); colonic immune responses were then analyzed. (A) Data analyses of DC responses. (B) Data analyses of T cell subset responses. A-B. Combined data from two independent experiments (n=3-7 mice/group). The P value was determined by a one-way ANOVA t-test followed by Turkey post-test. Error bars, S.E.M; *P<0.05, **P<0.01, ***P<0.001, ****P<0.0001.

FIGS. 20A-20D. Induction of immune responses and metabolic changes by P. UF1. GF mice were monoassociated with P. UF1 (green) or treated with PBS (white), and induced colonic immune responses were analyzed. (A) Analyses of DC responses. (B) Analyses of Th17 cell and Treg responses. (C-D) Metabolomic analyses of GF mice gavaged with P. UF1 or PBS (n=4-5 mice/group). (C) Heatmap of selected metabolites differentiating fecal samples of PBS-treated from P. UF1-treated GF mice. (D) Significant metabolic pathways of fecal samples from PBS-versus P. UF1-treated GF mice. Red dashed line shows permutation of P=0.05. A-B. Combined data from three independent experiments (n=14 mice/group). The P value in A and B was determined by a two-tailed unpaired t-test. Error bars, S.E.M; **P<0.01, ***P<0.001, ****P<0.0001.

FIGS. 21-21C. DlaT-specific Th17 cell differentiation. (A) Genetic organization of the dlaT locus from P. UF1 and ΔdlaT P. UF1 (left), PCR amplification of dlaT with primers P1/P2 (middle), and qRT-PCR analyses of the mRNA abundance of dlaT using primers P3/P4 (right) for identifying the ΔdlaT P. UF1. (B-C) GF mice were treated with P. UF1 (green), ΔdlaT P. UF1 (blue), or PBS (white); colonic immune responses were analyzed two weeks later. (B) Data analyses of DC responses. (C) Data analyses of Th17 cell and Treg responses. A. Representative data from three independent experiments (n=3 samples/group), B-C. Representative data from two independent experiments (n=6 mice/group). The P value in A was determined by a two-tailed unpaired t-test and in B-C by a one-way ANOVA t-test with Turkey post-test. Error bars, S.E.M; *P<0.05, **P<0.01, ***P<0.001.

FIGS. 22A-22F. Mitigation of proinflammation by P. UF1 during ΔactA L. m infection. (A-E) C57BL/6, RORγt^{-/-}, or Signr1^{-/-} mice were treated with P. UF1 (green) or with ΔdlaT P. UF1 (blue), and orally infected with either ΔactA L. m (red) or ΔactA L. m^{3pep} (grey). (A) Analyses of T cell subsets in C57BL/6 mice. (B) Analyses of tetramer DlaT²⁴⁵⁺ CD4⁺ T cells. (C) Analyses of T cell subsets in Signr1^{-/-} mice. (D-E) Microbiota analyses. (D) Taxonomic cladogram analyses of the experimental groups (P<0.05 by Kruskal-Wallis test). (E) Effect size of differentially abundant taxons in D; taxa enriched in ΔactA L. m (red), in P. UF1-treated+ΔactA L. m (green), in ΔdlaT P. UF1-treated+ΔactA L. m (blue), and ΔactA L. m^{3pep} (grey). (F) Heatmap of selected metabolites (P<0.05, t-test). A-B. Combined data from two independent experiments (n=7 mice/group); C. Representative data from two independent experiments (n=4-5 mice/group); D-F. Representative data from two independent experiments (n=3 mice/group). The P value in A and C was determined by one-way ANOVA t-test with Turkey post-test and in B by a two-tailed unpaired t-test. Error bars, S.E.M; *P<0.05, **P<0.01, ***P<0.001, ****P<0.0001.

FIG. 23. Microbiota analyses of two distinct cohorts of HBMF or FF preterm infants. Linear Discriminant Analysis Effect Size (LEfSe) represents the most differentially abundant taxons between HBMF and FF preterm infants' microbiota by day±13. Taxa enriched in HBMF preterm infant's microbiota have a negative score (blue), and taxa enriched in FF preterm infant's microbiota have a positive score (red). Only taxa with an absolute value of LDA score >2 are shown (n=20 samples/groups).

FIG. 24. Transient colonization of P. UF1 in C57BL/6 mice. Detection of P. UF1 in the fecal (black line) and the cecal samples (grey line) from C57BL/6 mice (n=2 mice/group) gavaged once with P. UF1 (10⁹ CFU/mouse). Representative data from two independent experiments. The P value was determined by a two-tailed unpaired t-test compared to day 0. Error bars, S.E.M; *P<0.05, **P<0.01, ***P<0.001.

FIGS. 25A-25E. Microbiota analyses of GF mice transfaunated with preterm infant fecal samples. (A-E) GF mice (n=3 mice/group) were orally transfaunated with HBMF or FF preterm infant fecal samples. A group of GF mice transfaunated with FF fecal samples was also treated with P. UF1 (10⁹ CFU/mouse). The composition of microbiota was analyzed 14 days later. (A) Summary box-plots of Chao Richness, Shannon diversity, and Pielou evenness indices derived from the results of 16S-based microbiota analyses. HBMF fecal samples (blue); FF fecal samples (red); and FF fecal samples+P. UF1 (green). (B) Mean total microbiota phyla composition of HBMF fecal samples, FF fecal samples, or FF fecal samples+P. UF1. (C) Taxonomic cladogram differences between the mouse groups. The significant taxons shown were filtered by LEfSe analyses (P<0.05 by Kruskal-Wallis test); blue: HBMF-enriched taxa, red: FF-enriched taxa, and green: FF+P. UF1-enriched taxa. (D) Three-dimensional unweighted PCA analyses of fecal microbiota. (E) LEfSe analyses represent the most differentially abundant taxons between mouse groups. Representative data from two independent experiments.

FIGS. 26A-26C. P. UF1 promotes DC and Th17 cell responses. (A-C) C57BL/6 mice (n=7 mice/group) were fed P. UF1 (10⁹ CFU/mouse) on days 0, 3, 6, 9 (4×). Two weeks later, mice were sacrificed and colonic cell responses were analyzed. (A) Representative data of DC responses. (B) Representative data of Th17 cell responses. (C) Representative data of Treg responses. A-C. Representative data from four independent experiments. The P value was determined by a two-tailed unpaired t-test. Error bars, S.E.M; *P<0.05, **P<0.01, ***P<0.001.

FIGS. 27A-27D. P. UF1 protects mice from Citrobacter rodentium infection. (A-D) C57BL/6 mice were orally infected with C. rodentium (10⁹ CFU/mouse, white dashed bars). A group of mice was gavaged with P. UF1 (10⁹ CFU/mouse, green) on days −1, 2, 5, and 8 (4×), with and without C. rodentium infection. Mice were sacrificed on day 14, and the colonic immune responses were analyzed. (A) Schematic schedule of P. UF1 treatment and representation of the initial weight loss percentage. (B) Representative data of DC responses. (C) Representative data of IL-17A⁺ or IFNγ⁺ CD4⁺ T cells, and Tregs. (D) Detection of C. rodentium in the fecal samples from days 4, 8, and 13 post-infection. A-D. Representative data from two independent experiments (n=5mice/group). Statistical significance for differences in weight loss in A was calculated using multiple unpaired t-tests correcting for multiple comparisons with the Holm-Sidak method. The P value in B-D was determined by a two-tailed unpaired t-test. Error bars, S.E.M; *P<0.05, **P<0.01, ***P<0.001.

FIGS. 28A-28D: Antigen-dependent Th17 cells by P. UF1. (A) CD4⁺ splenic cells from C57BL/6 mice gavaged with P. UF1 were transferred into H2-Ab1^{-/-} recipient mice. H2-Ab1^{-/-} mice were treated with P. UF1 (10⁹ CFU/mouse), or with PBS. Representative data of Th17 cell responses. (B) Mice were gavaged with P. UF1 or S-layer⁻ P. UF1. Representative data of Th17 cell and DC responses. (C) Splenic CD4⁺ T cells were magnetically isolated and labeled with CFSE. T cells were co-cultured with antigen-pulsed BMDCs (10:1) for five days. Cell proliferation was determined by CFSE dye dilution after five days. Representative histogram plots of CFSE of CD4⁺ IL-17A⁺ T cells. (D) C57BL/6 mice were gavaged with P. UF1. Splenic and MLN tetramer DlaT²⁴⁵⁺ cells were enriched with PE-beads. Representative plots of the percentage of DlaT²⁴⁵⁺ tetramer CD4⁺ T cells with a majority of IL-17A⁺ cells. A. Representative data from two independent experiments (n=8 mice/group). B. Representative data from three independent experiments (n=7 mice/group). C-D. Representative data from three independent experiments. The P value in A and B was determined by one-way ANOVA t-test with Turkey post-test. Error bars, S.E.M; *P<0.05, **P<0.01, ***P<0.001.

FIGS. 29A-29C. Transcriptome profiling. (A-C) Transcriptome profiling of P. UF1 (n=3) versus ΔdlaT P. UF1 cultured bacteria (n=3). (A) Unsupervised hierarchical clustering analyses on whole transcriptome of P. UF1 and ΔdlaT P. UF1 strains. (B) Heatmap presentation of selected genes associated with several pathways (filtered by P<0.05, NB model, Fold change >1.5). (C) Diagram of gene expression associated with tricarboxylic acid (TCA) cycle and pyruvate metabolism. Genes significantly upregulated in either condition (P<0.05, NB model, Fold change >1.5) are indicated with asterisk (*).

FIGS. 30A-30B. Metabolome profiling. (A-B) Metabolome profiling of P. UF1 (n=5) versus ΔdlaT P. UF1 (n=4) bacterial cultures. (A) Heatmap presentation of selected significant metabolites (P<0.005, t-test) differentially enriched in P. UF1 versus ΔdlaT P. UF1. (B) Pathway analyses using Mummichog 1.0.5. Significant pathways (permutation P<0.05) compared between P. UF1 and ΔdlaT P. UF1 strains are shown as bar chart.

FIGS. 31A-31C. Complementation of ΔdlaT P. UF1 with dlaT (P. UF1-1), the three dlaT peptides (P. UF1-2), or dlaT minus three DlaT peptides (P. UF1-3). (A) Schematic representation of P. UF1 strains: P. UF1-1 (orange), P. UF1-2 (purple), P. UF1-3 (black). (B) Detection by PCR of the dlaT gene or the three dlaT peptides in ΔdlaT P. UF1 after complementation with the aforementioned genes. (C) Relative mRNA abundance of the three dlaT peptides by qRT-PCR. B-C. Representative data from three independent experiments (n=3 samples/group). The P value was determined by a two-tailed unpaired t-test. Error bars, S.E.M; *P<0.05, **P<0.01, ***P<0.001.

FIGS. 32A-32B. Complementation of ΔdlaT P. UF1 with dlaT or the three dlaT peptides restores DC and T cell responses. (A-B) GF mice were gavaged with P. UF1-1 (10⁹ CFU/mouse) (orange), P. UF1-2 (purple), P. UF1-3 (black), or with PBS (white) on days 0, 3, 6, 9 (4×). Two weeks later, induced colonic immune responses were analyzed. (A) Representative data analyses of DC responses. (B). Representative data analyses of Th17 cells and Tregs. A-B. Combined data from two independent experiments (n=3-7 mice/group). The P value was determined by a one-way ANOVA t-test with Dunnett post-test, PBS-treated mice served as controls. Error bars, S.E.M; *P<0.05, **P<0.01, ***P<0.001, ****P<0.0001.

FIGS. 33A-33B. Mitigation of proinflammation by P. UF1 during L. m infection. C57BL/6 mice were gavaged with P. UF1 (green), or with ΔdlaT P. UF1 (blue) (10⁹ CFU) four times (4×). Mice were orally infected with L. m (10⁹ CFU) (red) or left uninfected (PBS) (white). Splenic immune responses were analyzed on day 7. (A) Representative data analyses of DC responses. (B) Representative data analyses of T cell subset responses. A-B. Combined data from two independent experiments (n=7-8 mice/group). The P value in A and B was determined by a one-way ANOVA t-test with Turkey post-test. Error bars, S.E.M; *P<0.05, **P<0.01, ***P<0.001.

FIGS. 34A-34D: Analyses of microbiota during L. m infection. (A) Summary box-plots of Chao Richness, Shannon diversity, and Pielou evenness indices derived from results of 16S microbiota analyses. L. m (red); P. UF1+L. m (green); ΔdlaT P. UF1+L. m (blue); and PBS (white). (B) Mean total microbiota phyla composition of L. m, P. UF1+L. m, ΔdlaT P. UF1+L. m, and PBS. (C) Taxonomic cladogram demonstrating taxonomic differences among mouse groups. (D) Effect size of the most differentially abundant taxons shown in graph C.

FIGS. 35A-35D. P. UF1 binds to SIGNR1. (A) Western blot of the purified SIGNR1-hFc fusion protein. (B) P. UF1 only binds to the extracellular domain of SIGNR1 (blue) but not to SIGNR3 (red), control fusion protein (yellow), or to secondary antibody (2^{nd} Ab) alone (green). (C) Relative expression of Signr1 and Signr3 in the distal colon from C57BL/6 mice gavaged with P. UF1 or PBS. (D) Representative data analyses of SIGNR1⁺ DCs in C57BL/6 mice or Signr1^{-/-} mice gavaged with P. UF1 or PBS. C-D. Representative data from three independent experiments (C: n=5 colonic tissue samples/group; D: n=8 mice/group). The P value in C and D was determined by a two-tailed unpaired t-test. Error bars, S.E.M; *P<0.05, **P<0.01.

FIGS. 36A-36B. SIGNR1 deficiency prevents the mitigation of proinflammation by P. UF1 during L. m infection. (A-B) Signr1^{-/-} mice were treated with P. UF1 (green) or with ΔdlaT P. UF1 (blue) four times (4×). Mice were orally infected with L. m (red) and 7 days later, splenic immune responses were analyzed. (A) Representative data analyses of DC responses. (B) Representative data analyses of T cell subsets. A-B. Combined data from two independent experiments (n=4-9 mice/group). The P value in A and B was determined by a one-way ANOVA t-test with Dunnett post-test, PBS-treated mice served as controls. Error bars, S.E.M; *P<0.05, **P<0.01.

FIGS. 37A-37H. L. m expressing DlaT peptides induces Th17 cell responses. (A) Schematic of L. m expressing the three DlaT peptides, L. m^{3pep}. (B) Confirmation of the 3 DlaT peptides secretion by L. m by Western blot. (C-F) C57BL/6 or Signr1^{-/-} mice were orally infected with 10⁹ CFU of L. m (red) or with L. m^{3pep} (grey) and 7 days post-infection, splenic immune responses were analyzed. (C and E) Representative data analyses of DC responses: C57BL/6 (C) or Signr1^{-/-} (E) mice. (D and F) Representative data analyses of T cell subset responses in C57BL/6 (D) or Signr1^{-/-} mice (F). (G-H) CFU of L. m recovered from the fecal samples or the liver, as indicated, of C57BL/6 mice (G) or Signr1^{-/-} mice (H) collected 7 days after infection. C-D. Combined data of two independent experiments (n=8 mice/group). E-F. Representative data from two independent experiments (n=4 mice/group). G-H. Representative data from two independent experiments (n=4 mice/group). The P value in C-F was determined by a two-tailed unpaired t-test and in G and H by a one-way ANOVA t-test with Turkey post test. Error bars, S.E.M; *P<0.05, **P<0.01, ***P<0.001.

FIGS. 38A-38D. Immune regulation by P. UF1 during ΔactA L. m infection. (A). Generation of ΔactA L. m expressing DlaT peptides, ΔactA L. m^{3pep}. (B) Confirmation of secretion of the 3 DlaT peptides by ΔactA L. m by Western blot. (C-D) C57BL/6 or Signr1^{-/-} (dashed bars) mice were treated with P. UF1 (green) or ΔdlaT P. UF1 (blue). Mice were orally infected with either 10⁹ CFU of ΔactA L. m (red) or with ΔactA L. m^{3pep} (grey). Colonic immune responses were analyzed 7 days later. (C) Representative data analyses of DC responses in C57BL/6 mice. (D) Representative data analyses of tetramer DlaT²⁴⁵⁺ CD4⁺ T cells and their positivity for IL-17A. C. Combined data from two independent experiments (n=7 mice/group). D. Combined data from two independent experiments (n=5mice/group). The P value in C was determined by a one-way ANOVA t-test with Turkey post-test and in D by a two-tailed unpaired t-test. Error bars, S.E.M; *P<0.05, **P<0.01, ***P<0.001.

FIGS. 39A-39B. Mitigation of proinflammation by P. UF1 during ΔactA L. m infection. (A-B) C57BL/6 or Signr1^{-/-} mice were treated four times (4×) with P. UF1 (green) or with ΔdlaT P. UF1 (blue), and orally infected with either ΔactA L. m (red) or ΔactA L. m^{3pep} (grey). (A) Representative dot plots of the bar graphs shown in the FIG. 22A. (B) Representative dot plots of the bar graphs shown in the FIG. 22C.

FIGS. 40A-40C. Immune regulation by P. UF1 during ΔactA L. m infection. (A-C) C57BL/6 or Signr1^{-/-} (dashed bars) mice were treated four times (4×) with P. UF1 (green) or ΔdlaT P. UF1 (blue). Mice were orally infected with either 10⁹ CFU of ΔactA L. m (red) or with ΔactA L. m^{3pep} (grey). Colonic immune responses were analyzed 7 days later. (A) Representative data analyses of DC responses in Signr1^{-/-} mice. (B-C) Determination of ΔactA L. m in fecal samples of C57BL/6 (B) or Signr1^{-/-} mice (C). A and C. Representative data from two independent experiments (n=4-5 mice/group). B. Combined data from two independent experiments (n=7 mice/group). The P value in A-C were determined by a one-way ANOVA t-test with Turkey post-test. Error bars, S.E.M; *P<0.05, **P<0.01, ***P<0.001.

### DETAILED DISCLOSURE OF THE INVENTION

The term “about” is used in this patent application to describe some quantitative aspects of the invention, for example, number of bacteria. It should be understood that absolute accuracy is not required with respect to those aspects for the invention to operate. When the term “about” is used to describe a quantitative aspect of the invention the relevant aspect may be varied by +10% (for example, 1%, 2%, 3%, 4%, 5%, 6%, 7%, 8%, 9% or 10%).

Potential benefit of P. freudenreichii supplementation of human donor milk or infant formula milk for feeding infants, for example, preterm infants, is provided. Significant differences between the microbiota composition of mother's milk-fed and formula milk-fed infants are shown. Among the differentially present bacteria, P. freudenreichii is more abundant in the mother's milk-fed group. Efficacy of P. freudenreichii supplementation in promoting favorable protective and regulatory immune responses is also demonstrated in experimental models of colitis. These same effects can be observed non-human mammals and nursed offspring of the non-human mammals.

Accordingly, the invention relates to the benefits provided to infants, for example, preterm infants, by feeding the infants human donor milk or infant formula milk supplemented with a single probiotic bacterium, for example, P. freudenreichii. The invention provides a method to improve the clinical outcome of infants, for example, preterm infants, by feeding them human donor breast milk or infant formula milk supplemented with a probiotic bacterium. The invention also provides a method to enhance the development of protective immune responses in infants that are able to breastfeed by feeding the mothers or a lactating female the probiotic bacterium during pregnancy and/or while breastfeeding. In various embodiments, the milk obtained from the mother or lactating female can be provided to an infant for bottle feeding or via breastfeeding by the mother or lactating female. Breast milk or milk obtained from a mother or lactating female to whom the composition disclosed herein has been administered may, optionally, be lyophilized or freeze-dried (or otherwise dehydrated) so as to form a powdered composition. The powdered composition can then be rehydrated using water or a buffer and administered to an infant as disclosed herein.

The invention also relates to benefits provided to human or non-human mammal offspring by feeding the offspring human donor milk or formula milk supplemented with a single probiotic bacterium, for example, P. freudenreichii. The invention also provides a method to enhance the development of protective immune responses in offspring of human and non-human mammals that are able to nurse by feeding the mothers or a lactating female the probiotic bacterium during pregnancy and/or while nursing. In various embodiments, the milk obtained from the mother or lactating female can be provided to an offspring for bottle feeding or via nursing by a mother or lactating female. Milk obtained from a mother or lactating female to whom the composition disclosed herein has been administered may, optionally, be lyophilized or freeze-dried (or otherwise dehydrated) so as to form a powdered composition. The powdered composition can then be rehydrated using water or a buffer and administered to an infant as disclosed herein.

In one embodiment, the probiotic bacterium is P. freudenreichii. Accordingly, the invention provides a method of feeding infants or non-human mammalian offspring, for example, preterm human infants, with compositions comprising P. freudenreichii. In one embodiment, the composition comprising P. freudenreichii is an infant food or food fed to newborn non-human mammalian offspring. Non-limiting examples of human infant foods include human donor milk and infant formula milk. Examples of milk or formula that can be administered to non-human offspring are also known in the art and can be supplemented with the probiotic bacterium is P. freudenreichii. Additional examples of infant foods of both human and non-human offspring are well known in the art and such embodiments are within the purview of the invention. Typically, P. freudenreichii supplemented milk or formula administered to a human or non-human mammalian offspring is suitable for the offspring and milk administered to an offspring is obtained from a lactating or nursing female of the same species.

In a further embodiment, P. freudenreichii is administered in a buffered aqueous solution, phosphate buffered saline (PBS). Other formulations of the invention suitable for oral administration of P. freudenreichii can be in the form of capsules, pills, tablets, powders, granules, or as a solution or a suspension in an aqueous or non-aqueous liquid, or as an elixir or syrup, etc., each containing a pharmaceutically effective amount of P. freudenreichii.

In one embodiment, the infant is a preterm infant. For the purpose of this invention, a preterm infant is an infant born before 37 completed weeks of gestation. A preterm infant can be born in the 23^{rd} to the 36^{th} week of gestation. Particularly, a preterm infant is born in the 23^{rd}, 24^{th}, 25^{th}, 26^{th}, 27^{th}, 28^{th}, 29^{th}, 30^{th}, 31^{st}, 32^{nd}, 33^{rd}, 34^{th}, 35^{th} or 36^{th} week of gestation. In other embodiments, the infant or non-human mammalian offspring nurses from the milk of a mother or surrogate providing milk to the offspring.

Various methods can be implemented in administering the compositions of the current invention to an infant or non-human mammalian offspring. For infants or offspring that are able to suck and/or swallow, the compositions can be fed to the infants or offspring. For infants or offspring that are not able to suck and/or swallow, the compositions can be administered by oral gavage, for example, via an orogastric tube.

The term “supplement”, “supplemented” or other related terms as used herein refer to adding a component (e.g. P. freudenreichii) to a composition. For example, supplementing an infant milk formula with P. freudenreichii indicates that the infant milk formula contains P. freudenreichii, for example, in a pharmaceutically effective amount in the infant milk formula. A further embodiment of the invention provides, a composition comprising a pharmaceutically effective amount of a probiotic bacterium, for example, P. freudenreichii. As discussed above, P. freudenreichii can be used as a supplement for the milk of any mammalian species that nurses offspring.

In one embodiment, the composition is an infant food, milk or formula used to nurse both human or non-human mammalian offspring. Non-limiting examples of human infant food are a donor breast milk and infant formula milk. Additional examples of infant foods are well known to a person of ordinary skill in the art and such embodiments are within the purview of the claimed invention. In another embodiment, the composition comprising the probiotic bacterium is a buffered aqueous solution, capsule, pill, powder, granule, solution or suspension in an aqueous or non-aqueous liquid, elixir or syrup.

The pharmaceutically effective amount of a bacterium varies with the subject to which the composition is administered. The pharmaceutically effective amount can be expressed as an absolute number, for example, colony forming units (CFU), or as a body weight based dosage, for example CFU/Kg of body weight of the subject. Typically, the pharmaceutically effective amount of the bacterium is about 10⁴ to about 10¹² CFU, about 10⁵ to about 10¹¹ CFU, about 10⁶ to about 10¹⁰ CFU, about 10⁸ to about 10¹⁰ CFU or about 10⁸ to about 10¹² CFU. In a specific embodiment, the pharmaceutically effective amount is about 10⁴ to about 10¹² CFU/Kg, about 10⁵ to about 10¹¹ CFU/Kg, about 10⁶ to about 10¹⁰ CFU/Kg, about 10⁸ to about 10¹⁰ CFU/Kg or about 10⁸ to about 10¹² CFU/Kg of the body weight of the subject to which the composition is administered. In one embodiment, the pharmaceutically effective amount is about 10¹² to about 10¹³ CFU/Kg of the body weight of the subject to which the composition is administered.

For the purposes of the invention “a pharmaceutically effective amount” refers to the amount of a bacterium, for example, P. freudenreichii, which is effective in producing beneficial effects in a subject ingesting the bacterium compared to an untreated subject. The beneficial effect can be the induction of regulatory and/or protective immune responses. In certain embodiments, the induced regulatory immune response includes, but is not limited to, reduced levels of pro-inflammatory cytokine, for example, IL-1β, TNF-α, IL-6, INF-γ; decreased frequency of IL-1β⁺ DCs; increased number of regulatory dendritic cells such as IL-10⁺ DCs; reduced IFNγ in T cells; increased Tregs in the MLNs and/or in the spleens (reduced IFNγ in T cells); increased FoxP3⁺ Tregs and decreased CD4⁺ and/or CD8⁺ T cells expressing IFNγ. The protective immune response includes, but is not limited to, immune responses which clear potential invading pathogens. In an even further embodiment, the beneficial effect is protection from colitis, for example, a dextran sulfate sodium (DSS)-induced colitis or a pathogen-induced colitis.

An effective amount of a bacterium is determined based on the intended goal. The term “unit dose” refers to a physically discrete unit suitable for use in a subject, each unit containing a predetermined quantity of the therapeutic composition calculated to produce the beneficial effect in association with its administration, for example, the appropriate route and treatment regimen. The quantity to be administered, both according to number of treatments and unit dose, depends on the subject to be treated, the state of the subject and the protection desired. Precise amounts of the therapeutic composition also depend on the judgment of the practitioner and are peculiar to each individual. Generally, the dosage of a bacterium will vary depending upon such factors as the patient's age, weight, height, sex, general medical condition and medical history. In specific embodiments, it may be desirable to administer the bacterium in the range of about 10⁴ to about 10¹² CFU/Kg, about 10⁵ to about 10¹¹ CFU/Kg, about 10⁶ to about 10¹⁰ CFU/Kg, about 10⁸ to about 10¹⁰ CFU/Kg or about 10⁸ to about 10¹² CFU/Kg of the subject's body weight. In one embodiment, the pharmaceutically effective amount is about 10¹² to about 10¹³ CFU/Kg of the subject's body weight.

In some embodiments of the invention, a pharmaceutically effective amount comprises administration of multiple doses of a bacterium. The pharmaceutically effective amount may comprise 1, 2, 3, 4, 5, 6, 7, 8, 9, 10, 15, 20, 25, 30, 35, 40, or more doses of a composition comprising a bacterium. In some embodiments, doses are administered over the course of 1 day, 2 days, 3 days, 4 days, 5 days, 6 days, 7 days, 10 days, 14 days, 21 days, 30 days, or more than 30 days. Moreover, treatment of a subject with a therapeutically effective amount of a bacterium can include a single treatment or can include a series of treatments. It will also be appreciated that the effective dosage of a bacterium used for treatment may increase or decrease over the course of a particular treatment.

Accordingly, a further embodiment of the invention provides a method of estimating a pharmaceutically effective amount of P. freudenreichii. The method comprises the steps of:
a. administering various amounts of P. freudenreichii to various test groups of subjects,
b. optionally, administering no P. freudenreichii or various amounts of a control bacterium to various control groups of subjects, and
c. determining the beneficial effect of various amounts of P. freudenreichii on the test groups of subject and determining the beneficial effect of no P. freudenreichii or various amounts of control bacterium on the control groups of subjects, and
d. comparing the beneficial effect of various amounts of P. freudenreichii within the test groups of subjects or with the beneficial effect of no P. freudenreichii or various amounts of control bacterium on the control groups of subjects, and
e. determining the amount of P. freudenreichii which produces the beneficial effect in the test groups of subjects as the pharmaceutically effective amount of P. freudenreichii.

In one embodiment the beneficial effect is development of beneficial microbiota in the intestines of the subjects. In another embodiment, the beneficial effect is induction of regulatory and/or protective immune responses. In certain embodiments, the induced regulatory immune response includes, but is not limited to, reduced levels of pro-inflammatory cytokine, for example, IL-1β, TNF-α, IL-6, INF-γ; decreased frequency of IL-1β⁺ DCs; increased number of regulatory dendritic cells such as IL-10⁺ DCs; reduced IFNγ in T cells; increased Tregs in the MLNs and/or in the spleens (reduced IFNγ in T cells); increased FoxP3⁺ Tregs and decreased CD4⁺ and/or CD8⁺ T cells expressing IFNγ. Where alterations in the number of cells is associated with a beneficial effect, standard techniques for quantifying cells can be used (e.g., fluorescent activated cell sorting (FACS), cytometry by time-of-flight (CyTOF) and confocal/two-photon imaging types). In a further embodiment, the protective immune response includes, but is not limited to, immune responses which clear potential invading pathogens.

The subject invention provides methods having both human and veterinary utility. The terms “individual” or “subject” includes mammals (i.e., humans and non-human mammals) that nurse newborns or juveniles with maternal milk, milk from surrogates or milk from other sources (e.g., formula). The term “offspring” is intended to encompass juvenile mammals that are nursed by a mother, surrogate or is formula fed and includes both human and non-human mammals that nurse and are nursed until the offspring is weaned from nursing. Non-human mammalian species which benefit from the disclosed methods include, and are not limited to, apes, chimpanzees, orangutans, monkeys; domesticated animals (pets) such as dogs, cats, guinea pigs, hamsters, Vietnamese pot-bellied pigs, rabbits, and ferrets; domesticated farm animals such as cows, buffalo, bison, horses, donkey, swine, sheep, and goats; exotic animals typically found in zoos, such as bear, lions, tigers, panthers, elephants, hippopotamus, rhinoceros, giraffes, antelopes, sloth, gazelles, zebras, wildebeests, prairie dogs, koala bears, kangaroo, opossums, raccoons, pandas, giant pandas, hyena, seals, sea lions, and elephant seals. Cytokine levels can be determined from a blood sample from a subject being tested, e.g., a serum or plasma sample, from a subject treated with a composition as disclosed herein. In various aspects of the invention, samples of the same type should be taken from both a control group (subjects not administered a composition as disclosed herein) and a treatment group (subjects receiving a composition as disclosed herein). In other aspects of the invention, a first sample is obtained from a subject before a composition, as disclosed herein, is administered to the subject and a second sample, of the same type as the first sample, is obtained from the same subject after the composition, as disclosed herein, is administered. The second sample can be obtained from the subject any period of time after administration of the composition to the subject (e.g., 3 days, 4 days, 5 days, 6 days, 7 days, 10 days, 14 days, 21 days, 30 days, or more than 30 days after the composition was first administered to the subject or after cessation of treatment of the subject with the composition).

For the purpose of monitoring cytokine levels in subjects treated as disclosed herein, a subject's blood samples may be taken at different time points before, during, and after the course of the therapy, such that the level of one or more cytokine can be measured to provide information indicating the effects of the treatment on the subject. Such monitoring may be performed repeatedly during several time periods (e.g., every 3 months, 6 months, 9 months, or every 12 months).

The serum or plasma of a blood sample from a subject is suitable for the present invention and can be obtained by well-known methods. For example, a blood sample can be placed in a tube containing EDTA or a specialized commercial product such as Vacutainer SST (Becton Dickinson, Franklin Lakes, N.J.) to prevent blood clotting, and plasma can then be obtained from whole blood through centrifugation. On the other hand, serum is obtained through centrifugation following blood clotting. Centrifugation is typically conducted at an appropriate speed, e.g., 1,500-3,000×g, in a chilled environment, e.g., at a temperature of about 4-10° C. Plasma or serum may be subject to additional centrifugation steps before being transferred to a fresh tube for measuring the level of a particular cytokine in the amount of protein. In some cases, the amount of mRNA may also be used to indicate the presence and quantity of a cytokine protein in the patient's blood using standard PCR techniques. In certain applications of this invention, plasma or serum may be the preferred sample types. In other applications of the present invention, whole blood may be preferable.

A cytokine, such as IL-1β, TNF-α, IL-6, IL-17A or INF-γ can be detected using a variety of immunological assays. In some embodiments, a sandwich assay can be performed by capturing the cytokine from a test sample with an antibody having specific binding affinity for the cytokine. The cytokine then can be detected with a labeled antibody having specific binding affinity for it. Such immunological assays can be carried out using microfluidic devices such as microarray protein chips. Cytokines can also be detected by gel electrophoresis (such as 2-dimensional gel electrophoresis) and Western blot analysis using specific antibodies. Alternatively, standard immunohistochemical techniques can be used to detect a cytokine protein, using the appropriate antibodies. Both monoclonal and polyclonal antibodies (including antibody fragment with desired binding specificity) can be used for specific detection of cytokine proteins. Such antibodies and their binding fragments with specific binding affinity to a particular cytokine can be generated by known techniques or obtained from commercial sources.

Other methods may also be employed for measuring cytokine level in practicing the present invention. For instance, a variety of methods have been developed based on the mass spectrometry technology to rapidly and accurately quantify target proteins even in a large number of samples. These methods involve highly sophisticated equipment such as the triple quadrupole (triple Q) instrument using the multiple reaction monitoring (MRM) technique, matrix assisted laser desorption/ionization time-of-flight tandem mass spectrometer (MALDI TOF/TOF), an ion trap instrument using selective ion monitoring SIM) mode, and the electrospray ionization (ESI) based QTOP mass spectrometer. See, e.g., Pan et al., J Proteome Res. 2009, 8(2):787-797.

In order to establish a control value of cytokine levels, a first group of subjects is selected (a control group). These individuals may optionally have the same gender, same or similar age, biological features (e.g., ethnic background), and/or medical history to be matched with the study (treated) group (subjects treated with a composition as disclosed herein). Furthermore, the number of subjects in the control group should be of a reasonable size, such that the average level of a cytokine obtained from the group can be reasonably regarded as representative of the average level of this cytokine among subjects that have not been treated with a composition as disclosed herein. Preferably, the control group includes at least 10 subjects. Typically, an average level of a given cytokine (e.g., average cytokine control value [the average cytokine levels from a control group]) is established for each distinct type of sample (e.g., serum sample, plasma, whole blood, etc.). Once an average cytokine control value is established for the level of a cytokine based on the individual values found in each individual of the control group, this value is considered a standard for the cytokine level for this type of sample. For instance, a cytokine level found in a plasma sample from a treated individual or treatment group should be compared with an average cytokine control value obtained from plasma samples.

In an even further embodiment, the beneficial effect is protection from colitis, for example, a DSS induced colitis or a pathogen induced colitis. In one embodiment, colitis is induced by C. rodentum. In certain embodiments, the pathogen causing colitis belongs to phyla: Firmucutes, Bacteroidetes, Actinobacteria or Proteobacteria. Non-limiting examples of colitis causing bacteria include Enterobacteriacea spp., Bacteroides fragilis, Pseudomonas aeruginosa, Bacteroides distasonis, B. vulgatus, Fuscobacterium varium and Clostridium spp. such as C. difficile. Additional examples of pathogens which cause colitis and which could be used in the methods of the current invention are well known to a person of ordinary skill in the art and such embodiments are within the purview of the invention.

In certain embodiments, various amounts of a bacterium administered to test and/or control subjects are about 10⁴ to about 10¹² CFU, 10⁵ to 10¹¹ CFU, 10⁶ to 10¹⁰ CFU, 10⁸ to 10¹⁰ CFU or 10⁸ to 10¹² CFU. The various amounts can be administered in multiple doses, for example, 1, 2, 3, 4, 5, 6, 7, 8, 9, 10, 15, 20, 25, 30, 35, 40, or more doses of a composition comprising a bacterium. In some embodiments, various amounts are administered over the course of 1 day, 2 days, 3 days, 4 days, 5 days, 6 days, 7 days, 10 days, 14 days, 21 days, 30 days, or more than 30 days.

An embodiment of the invention provides that P. freudenreichii useful for the methods described herein is P-UF1 as described in Example 6 below. The invention demonstrates that peptides of SEQ ID NOs: 1, 2, or 3 induce protective and/or regulatory immunity in a human or non-human mammalian offspring. A peptide comprising the sequences of SEQ ID NOs: 1, 2 and 3 also induces protective and/or regulatory immunity in a human or non-human mammalian offspring. SEQ ID NOs: 42-47 provide peptides comprising the sequences of SEQ ID NOs: 1, 2 and 3 arranged in six different ways.

Accordingly, a further embodiment provides a method of inducing protective and/or regulatory immunity in a human or non-human mammalian offspring, the method comprising administering a composition comprising one or more peptides selected from SEQ ID NO: 1, SEQ ID NO: 2, SEQ ID NO: 3, SEQ ID NOs: 42-47 or a combination thereof to the human or non-human mammalian offspring. The human offspring can be a preterm human infant and the composition can be an infant food, for example, donor milk or formula milk. In an embodiment the donor milk administered to a human or non-human mammalian offspring is obtained from a lactating female of the same species as the human or non-human mammalian offspring. Alternately, the composition comprising the peptides can be a buffered aqueous solution, capsule, pill, powder, granule, a solution or a suspension in an aqueous or non-aqueous liquid, an elixir or a syrup.

The composition comprising the peptides can be fed or gavaged to the human or non-human mammalian offspring.

Accordingly, a further embodiment provides a composition comprising an infant food and a pharmaceutically effective amount of one or more peptides selected from SEQ ID NO: 1, SEQ ID NO: 2, SEQ ID NO: 3, SEQ ID NO: 42-47 or a combination thereof, wherein the pharmaceutically effective amount of the one or more peptides induce protective and/or regulatory immunity in a human or non-human mammalian offspring. The composition of the peptides can be an infant food, a donor milk, a maternal milk. The infant food can be a solid infant food or infant formula.

Alternately, the composition of peptides can be a buffered aqueous solution, capsule, pill, powder, granule, a solution or a suspension in an aqueous or non-aqueous liquid, an elixir or a syrup.

Yet other embodiments a method of promoting development of intestinal microbiota and/or inducing protective and regulatory immunity comprising administering a composition comprising a pharmaceutically effective amount of a probiotic bacterium to a human or non-human. The probiotic bacterium can be P. freudenreichii, P-UF1 or a combination thereof. In various specific embodiments, the composition is administered to a preterm human infant, for example, a preterm human infant that has necrotizing entercolitis (NEC). In other embodiments, the composition is administered to a human having an inflammatory disease, such as inflammatory bowel disease, Crohn's Disease or ulcerative colitis. Yet other embodiments administer the disclosed composition to a human having an infection, such as a gastrointestinal infection. Another embodiment provides for the administration of the composition to an immunocompromised human (for example, a human infected with human immunodeficiency virus). The disclosed composition can also be administered to a human having cancer or humans over 50 years of age. As discussed above in relation to infants/offspring, the compositions can be administered in the form of a food in any of these embodiments.

This disclosure also provides methods of promoting development of intestinal microbiota and/or inducing protective and regulatory immunity comprising administering a composition comprising a pharmaceutically effective amount of one or more peptides selected from SEQ ID NO: 1, SEQ ID NO: 2, SEQ ID NO: 3, SEQ ID NOs: 42-48 or a combination thereof to a human or non-human. In various specific embodiments, the composition is administered to a preterm human infant, for example, a preterm human infant that has necrotizing entercolitis (NEC). In other embodiments, the composition is administered to a human having an inflammatory disease, such as inflammatory bowel disease, Crohn's Disease or ulcerative colitis. Yet other embodiments administer the disclosed composition to a human having an infection, such as a gastrointestinal infection. Another embodiment provides for the administration of the composition to an immunocompromised human (for example, a human infected with human immunodeficiency virus). The disclosed composition can also be administered to a human having cancer or humans over 50 years of age. As discussed above, the composition comprising a pharmaceutically effective amount of one or more peptides selected from SEQ ID NO: 1, SEQ ID NO: 2, SEQ ID NO: 3, SEQ ID NOs: 42-48 or a combination can be administered in the form of a food in any of these embodiments.

Another embodiment of the disclosed invention provides a recombinant bacterial cell comprising a vector, said vector comprising DNA operably linked to a promoter, said DNA encoding SEQ ID NO: 1, SEQ ID NO: 2, SEQ ID NO: 3, SEQ ID NOs: 42-47, SEQ ID NO: 48 or fragments of SEQ ID NO: 48. The recombinant bacterial cell can be a Propionibacterium strain, a Lactobacillus strain or Streptococcus thermophilus. In certain embodiments, the Lactobacillus strain is L. bulgaricus or L. lactis and the Propionibacterium strain is P. freudenreichii.

The disclosure also provides methods of promoting development of intestinal microbiota and/or inducing protective and regulatory immunity comprising administering a composition comprising a recombinant bacterial cell comprising a vector, said vector comprising DNA operably linked to a promoter, said DNA encoding SEQ ID NO: 1, SEQ ID NO: 2, SEQ ID NO: 3, SEQ ID NOs: 42-47, SEQ ID NO: 48 or fragments of SEQ ID NO: 48. As discussed above, the recombinant bacterial cell can be a Propionibacterium strain, a Lactobacillus strain or Streptococcus thermophilus. In certain embodiments, the Lactobacillus strain is L. bulgaricus or L. lactis and the Propionibacterium strain is P. freudenreichii. In various specific embodiments, the composition is administered to a preterm human infant, for example, a preterm human infant that has necrotizing entercolitis (NEC). In other embodiments, the composition is administered to a human having an inflammatory disease, such as inflammatory bowel disease, Crohn's Disease or ulcerative colitis. Yet other embodiments administer the disclosed composition to a human having an infection, such as a gastrointestinal infection. Another embodiment provides for the administration of the composition to an immunocompromised human (for example, a human infected with human immunodeficiency virus). The disclosed composition can also be administered to a human having cancer, humans over 50 years of age or humans, in general.

### Materials and Methods

Subjects and Fecal Sample Collection

Preterm infants with gestational ages less than or equal to 32 completed weeks and birth weights less than or equal to 1,800 grams were eligible for the study. Infants with major congenital anomalies or malformations were excluded. Stool samples were collected weekly from the study infants starting with meconium and continued until discharge. The samples were immediately stored at −80° C. The analyzed samples were derived from control infants who did not develop necrotizing enterocolitis (NEC) or culture positive for sepsis (Table 1). Two groups, each with 20 subjects, were selected and either received a human breast milk fed (HBMF) diet or a formula fed (FF) diet. For each group, subject samples from two different time points were analyzed: the first group of samples was collected at 13±2-3 days after birth with 11±3 days of feeding, and the second group of samples was collected at 21±3 days after birth with 19±4 days of feeding.

TABLE 1
Selected preterm infant cohorts.

| | Breast milk | Formula |
|---|---|---|
| Gestational Age (weeks) | 29 ± 2 | 30 ± 2 |
| Birth Weight (grams) | 1198 ± 383 | 1360 ± 436 |
| Caucasian | 80% | 35% |
| Vaginal Delivery | 25% | 45% |
| Chorioamnionitis | 20% | 60% |
| Male | 70% | 50% |
| Maternal Antibiotics | 90% | 85% |
| Infant Antibiotics | 80% | 85% |

Animals

C57BL/6 and H2-Ab1^{-/-} (B6.129S2-H2^{dlAb1-Ea}/J) mice were obtained from Jackson Laboratory. Signr1^{-/-} mice and RORγt^{-/-} mice were kindly provided. C57BL/6 germfree (GF) mice were maintained until the experiments were performed. Mice were maintained under specific pathogen-free, Helicobacter-free conditions and used at 6-8 weeks of age.

Preterm Infant Microbiota Analyses and Fecal Microbiota Transfer into GF Mice

Microbiota analyses were performed on the Illumina Miseq (Illumina, Inc., San Diego, Calif.). Sequence analyses were performed using QIIME v.1.9.0. Briefly, after checking quality of sequenced reads, 8 nucleotide (nt) barcodes were extracted from both forward and reverse reads to generate a barcode library. Forward and reverse reads were then joined. Sequence libraries were filtered, and split based on their corresponding barcodes. An open reference operational taxonomic unit (OTU) picking strategy was used to select OTU (with 97% identity threshold). Taxonomy was assigned based on the Greengenes reference database. After single alpha rarefaction based on rarefaction curve, alpha diversity (e.g., Chao diversity, Shannon diversity, Evenness index) was measured. Subsequently, a taxonomic table for each taxonomic level was generated based on the OTU table. The phylum level taxonomic table was the basis for the phylum pie chart depiction. Differentially significant features at each level were identified using linear discriminant analysis (LDA) along with effect size measurements (LEfSe). The significant taxons were filtered by LDA Effect Size (LEfSe) analyses with default criteria (p<0.05 by Kruskal-Wallis test; LDA score >2). The LEfSe data were further used for cladogram depiction using Graphlan. For the studies involving transfaunation of GF mice, pooled fecal samples collected from preterm HBMF or FF infants were dissolved in reduced sterile PBS, and animals subsequently gavaged with 100 μL of the fecal slurry on day 0. A group of GF mice was gavaged every third day with P. UF1 (10⁹ CFU/mouse) on days −1, 2, 5 and 8.

First Draft Genome Analyses of P. UF1

Fecal samples of HBMF preterm infants (100 mg) were dissolved in sterile lectin-binding buffer and biotinylated Concanavalin A (ConA, 100 μg) (Vector Laboratories, Burlingame, Calif.) was added to the suspension. The fecal contents were incubated at 4° C. for 3 hr and subsequently washed with lectin-binding buffer three times. The Streptavidin-ferrous beads (Thermo Fisher Scientific, Waltham, Mass.) were then added to the washed fecal contents allowing the ConA-bacteria complex to bind to the beads. The ferrous beads were washed six times with lectin-binding buffer in the presence of a magnet. The enriched bacteria, via bead complex, were spread over several de Man, Rogosa, and Sharpe (MRS)-lactate agar plates (MRS agar with 1% sodium lactate; w/v) to grow the separated bacteria for three days. Bacterial DNA of the colonies was isolated, and bacterial identity was determined by the amplification of 16S rRNA and sequencing analyses. One of the isolates was designated as P. UF1. Genomic DNA of P. UF1 was isolated, purified and subjected to PACBIO RS II for whole genome sequencing. The sequenced contigs by PACBIO RS II were assembled into one unique contig, by using SMRT Portal, which was then compared with known sequences of P. freudenreichii subsp. shermanii CIRM-BIA1 and of P. freudenreichii subsp. freudenreichii DSM20271T. A distinguishable prophage identified by PHAST (Phage Search Tool) was also shown in the comparison to demonstrate the key bacterial strain differences and the missing gaps visualized in a circular genome map. The circular map was generated using Blast Ring Image Generator (BRIG). The genome of the P. UF1 was annotated and combined using Rapid Annotation Server (RAST), KEGG Orthology And Links Annotation (KOALA), and Rapid Prokaryotic Genome Annotation (PROKKA). The subsystem categories were summarized and visualized by using RAST.

Bacterial Strains and their Products

Escherichia coli DH5a was used for plasmid construction and grown in Luria-Bertani (LB) medium at 37° C. The P. UF1 and its genetically modified strains were grown at 30° C. in MRS medium (Difco Laboratories, Detroit, Mich.) supplemented with 1% (w/v) sodium lactate (Thermo Fisher Scientific, Rockford, USA) in an anaerobic chamber (Model AS-580, Anaerobe Systems, Morgan, Hill, Calif.). The transformations of the P. UF1 were performed. The Listeria monocytogenes (L. m) strains and Citrobacter rodentium were grown in Brain Heart Infusion (BHI) (Difco Laboratories, Detroit, Mich.) medium at 37° C. Antibiotics were added at the following final concentrations: 5 μg/mL chloramphenicol and 1 mg/mL hygromycin B for P. UF1 strains; 200 μg/mL streptomycin and 50 μg/mL kanamycin for L. m. Surface layer proteins (Slp) were extracted from P. UF1 cultures by resuspension in guanidine hydrochloride (4 M) at 37° C. for 30 min. The supernatants containing Slp were dialyzed using a 10 kDa cutoff Slide-A-Lyer® Dialysis Cassette (Thermo Fisher Scientific, Rockford, USA). The brushed off bacteria obtained during this process was assigned as S-layer⁻ P. UF1.

Peptide Identification

The S-layer of P. UF1 and S-layer⁻ P. UF1 were separated by SDS-PAGE and analyzed by mass-spectroscopy (MS). Dihydrolipoamide acyltransferase (DlaT) was one of the major S-layer proteins from which three 15-mer amino acid peptides, including DlaT 208-222 (VGSSVPAAPAAAPAA, SEQ ID NO: 1), DlaT 245-259 (VAPPAPVAPSAPVAA, SEQ ID NO: 2), and DlaT 109-123 (AAAPVAPPAPPAPAA, SEQ ID NO: 3), were selected and synthesized (Thermo Fisher, Ulm, Germany).

Generation of P. UF1 Strains

The insertional inactivation of the dlaT gene in P. UF1 was performed by a single crossover strategy. Briefly, a 516-bp internal fragment of dlaT was amplified from the P. UF1 genome using primers DlaT-F/DlaT-R (Table 2). The chloramphenicol resistance gene (cmR, 1,512-bp) was synthesized by GenScript Corporation (Piscataway, N.J.). The resulting two fragments were cloned into a pUC18 plasmid, yielding suicide plasmid, pUCC-dlaT. Following transformation into P. UF1, the chloramphenicol resistant colonies were selected, and the ΔdlaT P. UF1 was confirmed by sequencing the dlaT locus amplified with primers P1/P2. The stability of the mutants was ascertained under antibiotic-free conditions. For gene complementation, a novel shuttle vector (pYMZ) between E. coli and P. UF1 was first constructed. Briefly, a hygromycin B (hygB) resistance gene (1128 bp) and a pLME108 replicon (2,063 bp) were synthesized and cloned into a pUC18 plasmid, resulting in the pYMZ plasmid. A fragment (2,145 bp) containing the intact dlaT gene and its native promoter (P_{dlaT}) was amplified from the P. UF1 chromosome using CdlaT-F/CdlaT-R primers, digested with XbaI/BamHI, and cloned into the pYMZ plasmid to generate pYMZ-dlaT. To complement the three DlaT peptides (3pep), a fragment (507 bp) encoding the 3pep was amplified with 3Pep-lap-F/3Pep-R primers, and the dlaT promoter region (361 bp) was amplified with CdlaT-F/3Pep-lap-R primers. The P_{dlaT}-3pep fragment was generated by overlap PCR, digested with XbaI/KpnI, and cloned into the pYMZ plasmid yielding pYMZ-3pep, which overexpressed the 3pep under control of the native dlaT promoter. The dlaTΔ3pep fragment was generated by several overlap extension PCRs. CdlaT-F/Pep1-lap-R and Pep1-lap-F/CdlaT-R primers were used to amplify the fragments using overlap extension PCR to generate fragment, dlaTΔpep1. PCR amplifications utilizing the CdlaT-F/Pep2-lap-R and Pep2-lap-F/CdlaT-R primers and the template, dlaTΔpep1, resulted in the fragment, dlaTΔpep1pep2. Finally, dlaTΔ3pep was generated by overlap PCR using CdlaT-F/Pep3-lap-R and Pep3-lap-F/CdlaT-R primers, and the dlaTΔpep1pep2 template. The purified dlaTΔ3pep fragment was digested with XbaI/BamHI and cloned into the pYMZ plasmid, generating pYMZ-dlaTΔ3pep. Plasmids, including pYMZ-dlaT, pYMZ-3pep, and pYMZ-dlaTΔ3pep, were electroporated into the ΔdlaT P. UF1 strain, resulting in transgenic strains, P. UF1-1, P. UF1-2, and P. UF1-3, respectively. All of the complementation strains were confirmed by restriction digestion and DNA sequencing of the plasmids isolated from corresponding recombinant P. UF1 strains.

TABLE 2
Oligonucleotides used in this study.

| Oligonucleotide | Sequence (5′ to 3′) | SEQ ID NO: |
|---|---|---|
| DlaT-F | CCCAAGCTTGAGGTGGCTGAAGGAAGTTG | 5 |
| DlaT-R | CCCGGATCCTCCTCCTTGACGTGGATCTC | 6 |
| CdlaT-F | CCCTCTAGACCAGCTTCTCGTGACACTCATTC | 7 |
| CdlaT-R | CCCGGATCCCGCGATCCCCGTTACGG | 8 |
| 3Pep-lap-R | CGGCTGCGGGAATCGGCATGGTTCTACGTGACT | 9 |
| | CCTTTG | |
| 3Pep-lap-F | CAAAGGAGTCACGTAGAACCATGCCGATTCCCG | 10 |
| | CAGCCG | |
| 3Pep-R | CCCGGATCCCTAAGCCGGTGGGTTCACCG | 11 |
| Pep1-lap-F | CCCCGATTCCCGCAGCCGCGGCACCTGCCGGC | 12 |
| Pep1-lap-R | GCCGGCAGGTGCCGCGGCTGCGGGAATCGGGG | 13 |
| Pep2-lap-F | CCGTACTCGGCGTCGTCCCTGCGGCACCCGCC | 14 |
| Pep2-lap-R | GGCGGGTGCCGCAGGGACGACGCCGAGTACGG | 15 |
| Pep3-lap-F | GCGGCTCCGGCCCCCCCGCCCGCGGCGCCG | 16 |
| Pep3-lap-R | CGGCGCCGCGGGCGGGGGGGCCGGAGCCGC | 17 |
| 3Pep-F2 | CAACAAACTGAAGCAAAGGATGTTGGTAGTAGT | 18 |
| | GTCCCA | |
| 3Pep-R2 | ATATGTCGACCTATGCAGCAACTGGTGCA | 19 |
| HlySS-F | ATATCCATGGCCAAAAAAATAATGCTAGTTTTT | 20 |
| | ATTA | |
| HlySS-R | TGGGACACTACTACCAACATCCTTTGCTTCAGTT | 21 |
| | TGTTG | |
| Signrlexp-F | CCGGAATTCGCAAGTCTCCAAAACCCCA | 22 |
| Signrlexp-R | CATGCCATGGGGCCTTCAGTGCATGGGGTTGC | 23 |
| C. rodentium-F | AAGTCTGTCAATACCGCCTC | 24 |
| C. rodentium-R | AATGTGCCAACTGTCTCATC | 25 |
| P1 | GTCTTCTGCCCCATACGCTAA | 26 |
| P2 | GATGTCCTCGGAGTCGTGGTA | 27 |
| P3 | CACCCTGACGGAGATCCAC | 28 |
| P4 | CCGACGACGCCGAGTAC | 29 |
| GroL2-RT-F | CAATGTCGTGTTGGAGAAG | 30 |
| GroL2-RT-R | CGCCGATCTTGTGGTAGG | 31 |
| Gapdh-RT-F | GGTGAAGGTCGGTGTGAACG | 32 |
| Gapdh-RT-R | CTCGCTCCTGGAAGATGGTG | 33 |
| Signr1-RT-F | TTGATGGTCAGCGGCAGCAGG | 34 |
| Signr1-RT-R | TCAGCAGGAGCCCAGCCAAGA | 35 |
| Signr3-RT-F | GGGCCCAACTGGTCATCATA | 36 |
| Signr3-RT-R | AGCGTGTAAAGCTGGGTGAC | 37 |
| rDNA-F | AGGATTAGATACCCTGGTA | 38 |
| rDNA-R | CRRCACGAGCTGACGAC | 39 |

RNA Isolation, Reverse-Transcription PCR (RT-PCR), and Quantitative PCR (qPCR)

Total RNA was extracted from P. UF1 bacterial culture (1 mL, OD₆₀₀=0.8) or ˜0.5 cm length of distal colons using the RNA isolation kit (Roche, Indianapolis, Ind.), according to the manufacturer's instructions. DNA contaminants were completely removed by an additional digestion with DNase I (Roche, Indianapolis, Ind.), and the cDNA was synthesized using an RT kit (Roche, Indianapolis, Ind.) with random primers (Roche, Indianapolis, Ind.). For measuring the relative expression of dlaT in P. UF1 and of Signr1 in the colonic tissues, qRT-PCR was performed using groL2 (for P. UF1) or Gapdh (for colonic tissue) as the reference genes. Briefly, cDNA samples (2 μL) were added to SYBR green master reaction mixtures (18 μL) (Bio-Rad, Hercules, Calif.) containing primers (0.5 μM of each) GroL2-RT-F/GroL2-RT-R (for groL2), primers P3/P4 (for dlaT or 3pep), Signr1-RT-F/Signr1-RT-R (for Signr1), Signr3-RT-F/Signr3-RT-R (for Signr3), or Gapdh-RT-F/Gapdh-RT-R (for Gapdh). For each sample, qRT-PCR was assayed in technical triplicate, and for each reaction, the calculated C_{T} value was normalized to the C_{T} of groL2 or Gapdh amplified from the corresponding sample. The relative mRNA levels were calculated using the 2^{-ΔΔCT} comparative method, and the results were obtained from the analyses of at least three samples in triplicate.

Generation of L. m Secreting the Three DlaT-Derived Peptides

The nucleotide sequence corresponding to the three DlaT peptides was synthesized and amplified with primers, 3Pep-F2/3Pep-R2. The L. m secretion signal sequence of the hly gene (encodes Listeriolysin 0) was amplified with primers, HlySS-F/HlySS-R. The resulting two amplicons were used as templates in a splicing by an overlap extension PCR reaction to generate a DNA fragment containing the secretion signal sequence of the hly gene directly upstream of the 3pep sequence. This fragment was then cloned into the L. m integration vector, pIMK2, which drives gene expression under the control of the highly active P_{HELP} promoter, generating plasmid, pIMK2-3pep. This plasmid was then electroporated into L. m 10403S harboring two amino acid substitutions in the bacterial invasion protein, Internalin A (InlA^{m}-S192N, Y363S), thereby rendering the bacterium more efficient at invading murine gut epithelial cells. For experiments using ΔactA L. m (renders the bacterium incapable of cell-to-cell spread by preventing bacterial-mediated host actin polymerization), standard allelic replacement methods were used to generate murinized InlA^{m} in the background of a ΔactA mutant, followed by introduction of pIMK2-3pep by electroporation. To confirm that L. m strains 10403S InlA^{m} and 10403S InlA^{m}ΔactA, harboring a stably integrated pIMK2-3pep, were capable of expressing and secreting the recombinant DlaT peptides, an additional pIMK2-3pep construct containing a 6×-Histidine tag at the C-terminus (plasmid pIMK2-3pep-6×His) was generated for immunodetection. The supernatants of the bacterial cultures were collected, precipitated with 10% trichloroacetic acid, washed with 1 mL of 100% ethanol, and dissolved in SDS sample buffer. The protein samples were run on an 18% SDS-PAGE gel, transferred to a PVDF membrane, and detected by Western blot using an anti-6×His mouse monoclonal antibody (Abcam, Cambridge, Mass.).

Gavaging Mice with P. UF1 Strains

Mice were gavaged with P. UF1 strains four times (4×) every 3 days, and the immune responses analyzed on day 14. Mice received P. UF1 (10⁹ CFU/100 μL PBS), or its derivative strains (10⁹ CFU/100 μL PBS), including ΔdlaT P. UF1, P. UF1-1, P. UF1-2, P. UF1-3, or S-layer⁻ P. UF1.

Bacterial Infections

Mice were gavaged with either P. UF1, ΔdlaT P. UF1, or PBS on days −1, 2, 5, and 8 for Citrobacter rodentium infection and on days −7, −4, −1, and +2 for L. m relative to the infection. C. rodentium infection: mice were orally infected after 4 hr of fasting with C. rodentium (10⁹ CFU/mouse) on day 0 and then sacrificed on day 14. The presence of C. rodentium in the fecal samples of C57BL/6 mice was determined by qRT-PCR using C. rodentium-specific primers. L. m infection: mice were denied food, but given unrestricted access to water 8 hr prior to infection. The L. m inoculum was suspended in 100 μL PBS with CaCO₃ (50 mg/ml). Mice were orally infected with L. m (10⁹ CFU/mouse) on day 0. Mice were then monitored daily and sacrificed on day 7. Fecal samples were collected on days 2, 4, and 7. Livers were also collected, weighed, and suspended in sterile PBS on day 7. Subsequently, the supernatants were diluted and plated on BHI agar (200 μg/mL streptomycin). Colonies were counted at 37° C. after 24-36 hr of growth.

Cell Suspensions

Colonic lamina propria cells were isolated. Enriched splenic cells were prepared by depleting B cells using plates coated with anti-mouse IgG (H+L) (Jackson ImmunoResearch Laboratories, Inc., West Grove, Pa.) at 37° C. for 30 min. The non-adherent cells were subjected to CD4⁺ T cell Isolation Kit II (MACS, Miltenyi Biotec, San Diego, Calif.). The purity of the obtained cells was analyzed by flow cytometry and determined to be >98%, from which cells (1×10⁶) were injected intraperitoneally (i.p.) into the H2-Ab1^{-/-} recipient mice.

Flow Cytometry

For cytokine analyses, cells were stimulated with 50 ng/mL phorbol 12-myristate 13-acetate (PMA) and 2.5 μg/mL ionomycin in the presence of brefeldin A (Sigma-Aldrich, St. Louis, Mo.) for 2.5 hr for the colons and 4 hr for the spleens and mesenteric lymph nodes (MLNs) at 37° C. Cell surface staining was carried out in PBS with 1% BSA, 2 mM EDTA, and 0.1% sodium azide. For live cell analyses, dead cells were excluded by staining with LIVE/DEAD® Fixable Blue Dead Cell Stain Kit (Molecular Probes, Thermo Fisher Scientific, Waltham, Mass.). Cells were incubated with Mouse Fc Blocking Reagent (Miltenyi Biotec, Auburn, Calif.) prior to staining. Cells were first stained for cell-surface markers and then resuspended in fixation/permeabilization solution [Cytofix/Cytoperm kit (BD Biosciences, San Jose, Calif.) or FOXP3 Fix/Perm buffer set (Biolegend, San Diego, Calif.) for FoxP3 intracellular staining)]. Intracellular staining was carried out in accordance with the manufacturer's instructions. Data were collected by a LSR Fortessa (BD Biosciences, San Jose, Calif.) and data were analyzed with FlowJo software (TreeStar, Ashland, Oreg.). Antibodies for staining were purchased from BD Bioscience (San Jose, Calif.), Biolegend, eBioscience (San Diego, Calif.) or R&D Systems (Minneapolis, Minn.). The following antibodies or their corresponding isotype controls were used: CD45 (30-F11), CD11c (N418), CD11b (M1/70), F4/80 (BM8), GR1 (RB6-8C5), I-A/I-E MHCII (2G9), CD3 (145-2C11), CD4 (RM4-5), CD8 (53-607), Pro-IL-1β (NJTEN3)/Rat IgG1, κ, IFNγ (XMG1.2)/Rat IgG1, κ, IL-17A (TC11-18H10.1)/Rat IgG1, κ, IL-10 (JES5-16E3)/Rat IgG2b, κ, FoxP3 (FJK-16A)/Rat IgG2a, κ, RORγt (AFKJS-9)/Rat IgG2a, κ, IL-17A (eBio187)/Rat IgG1 K, TGF□ (TW7-16B4)/Rat IgG1, κ, IL-12 (C15.6)/Rat IgG1, κ, IL-6 (MP5-20F3)/Rat IgG1, κ, SIGNR1 (22D 1)/Hamster IgG.

DC and T Cell Co-Cultures

Bone marrow DCs (BMDCs) were cultured, as previously described. BMDCs were pulsed with specific DlaT peptides (20 μg/mL), or Slp (10 jag/ml), as a control, for 12 hr. For proliferation assays, CD4⁺ T cells were labeled with 10 μM carboxyfluorescein succinimidyl ester (CFSE), according to the manufacturer's instructions (Molecular Probes, Waltham, Mass.), and co-cultured with peptide pulsed BMDCs. Each in vitro experiment was conducted in triplicate with splenic CD4⁺ T cells pooled from three C57BL/6 mice that were gavaged four times with P. UF1. BMDCs (5×10⁴) were co-cultured with CD4⁺ T cells at the ratio of 1:10 for 5 days. Cells were stained with a panel of antibodies and analyzed by LSR Fortessa flow cytometry.

Tetramer Analyses

MHC II tetramers presenting DlaT and control peptides were custom prepared. The following PE-labeled tetramers were generated: I-A(b) DlaT 208-222 (SEQ ID NO: 1), I-A(b) DlaT 245-259 (SEQ ID NO: 2), I-A(b) DlaT 109-123 (SEQ ID NO: 3), and control tetramer (PVSKMRMATPLLMQA, SEQ ID ON: 4). MLN, splenic, and colonic cells from mice gavaged with P. UF1 were stained with I-A(b) DlaT 245-259 (SEQ ID NO: 2) (Tetramer DlaT²⁴⁵ showed significant binding to MHC II molecules) for 1 hr at 4° C. For MLN and splenic cells, a magnetic enrichment step was performed. Cells were labeled with extracellular antibodies and then treated with fixation/permeabilization buffer (Biolegend, San Diego, Calif.) for intracellular staining.

SIGNR1-hFC Binding Assay

SIGNR1, SIGNR3, and a control peptide were fused to the human Fc (hFc) part of human IgG1 (SIGNR1-hFc; SIGNR3-hFc; Ctrl-hFc). Briefly, the cDNA encoding for the extracellular part of SIGNR1 and SIGNR3 was amplified using Signr1exp-F/Signr1exp-R primers. The product was fused in frame to the Fc region of human IgG1 by cloning into EcoRI and NcoI sites of the plasmid, pINFUSE-hIg1-Fc2 (InvivoGen, San Diego, Calif.). Chinese hamster ovary (CHO) cells were transfected by a plasmid containing SIGNR1, and supernatants were collected two days later. The fusion proteins were purified using protein G columns (Thermo Fisher Scientific, Waltham, Mass.), according to the manufacturer's protocol. Purified SIGNR1-hFc and SIGNR3-hFc fusion proteins were confirmed by Western blot using anti-SIGNR1 and anti-SIGNR3 antibodies (R&D Systems, Minneapolis, Minn.). Protein G column purified proteins were used for binding to P. UF1.

Gut Transient Colonization of P. UF1

C57BL/6 mice were gavaged with P. UF1 (10⁹ CFU/mouse) and fecal samples were collected every day. Every day, mice (n=2) were sacrificed to collect cecal and fecal samples. Bacterial genomic DNA was isolated from the fecal samples and cecal contents using a Zymo Fecal DNA MiniPrep kit (Zymo Research, Irvine, Calif.). Fecal DNA (10 ng) was added to qPCR mixtures containing either P. UF1-specific primers, P3/P4, or 16S rDNA universal primers, rDNA-F/rDNA-R. The percentage of P. UF1 in the total bacteria was expressed as the percentage of dlaT copies normalized to the copies of 16S rDNA.

Bacterial Transcriptome Analyses

Total RNA was extracted from P. UF1 and ΔdlaT P. UF1 strains (3 replicates of each strain), and DNA contaminants were removed by an Ambion TURBO DNA-free kit (Thermo Fisher Scientific, Waltham, Mass.). The ScriptSeq Complete Gold Kit (Illumina, Inc., San Diego, Calif.) was used for rRNA depletion and the cDNA library generation. Subsequently, cDNA libraries were evaluated, pooled, and sequenced on the Illumina MiSeq machine, which generates 7-8 million paired-end reads/sample (˜75 nt). The sequence reads first underwent data preprocessing and quality evaluation, including trimming off the low quality ends of reads. Rockhopper was used to map sequence reads to the annotated P. UF1 genome, to normalize the counts values by upper quartile normalization, and to perform differential expression analyses using the negative binomial (NB) statistical model. Principle component analyses (PCA) were performed based on normalized counts to visualize the separation between groups using python. Selected genes were visualized in a heatmap after Z-score transformation upon normalized counts across all samples in each gene (filtered by p<0.05, NB model; Fold change >1.5). Diagrams of gene expression associated with the TCA cycle and pyruvate metabolism were presented.

Ultra-High Resolution Metabolomic Analyses

Mouse fecal samples and P. UF1 bacterial cultures were collected and frozen to be analyzed. The samples were processed by acetonitrile (2:1, v/v), followed by centrifugation, before being analyzed by a high-resolution mass spectrometer coupled with liquid chromatography (LC-MS). The fecal samples were analyzed on a LTQ-FTICR mass spectrometer and the bacterial samples on an Orbitrap Fusion Tribrid Mass Spectrometer (Thermo Fisher, San Diego, Calif.). Each sample was added spike-in controls and run in triplicates; standard reference samples were run in the beginning and end of each batch. An in-house informatics pipeline was used to perform peak detection, noise filtering, mass to charge (m/z) ratio and retention time alignment, and feature quantification. Subsequently, the feature table was subjected to quality assessment, including exclusion of data for technical replicates with overall Pearson correlation (r)<0.70. Feature values (MS peak intensities) of each sample were summarized by the average of filtered non-zero technical replicates, normalized by total ion intensity, and log 2 transformed. For each experiment, significant metabolite features were selected by t-test or ANOVA, and subjected to pathway analyses with the Mummichog software. In pathway analyses of fecal samples from control GF treated with PBS versus P. UF1-monoassociated GF mice, 363 significant metabolite features (P<0.05, t-test) were used as Mummichog input. In the analyses of bacterial samples from P. UF1 versus ΔdlaT P. UF1, 389 significant metabolite features (p<0.005, t-test), were used as Mummichog input. Only adduct ions with [M+H]⁺ and [M+Na]⁺ were chosen for visualization in a heatmap with Z-score transformed values presented. Among the metabolites, arginine, citrulline, betaine/valine, tryptophan, phenylalanine, methionine lysine, aspartate, tyrosine and creatine were confirmed by chemical standards, while others were based on high-confidence database matches (<10 ppm).

Statistical Analyses

Most statistical analyses were performed using GraphPad Prism (Version 6.0 for Mac OS X, La Jolla, Calif.). Mean and S.E.M. values and statistical significance between two variables were determined by a two-tailed unpaired t-tests. Where appropriate, one-way ANOVA followed by Dunnett (PBS group used as control), Turkey or Bonferroni post-tests was performed. Differences were considered to be significant at *p<0.05, **p<0.01, ***p<0.001, **** p<0.0001. For microbiota analyses, the Kruskal-Wallis test was used in LEfSe to perform statistical testing for microbiota compositions. Differences were considered to be significant at p values <0.05 and only features with an LDA score >2 were shown. For tests in differential gene expression analyses, the negative bionomial (NB) model was applied as the statistical model to compute p values, followed by the Benjamini-Hochberg procedure to calculate q-values. For metabolomics analyses, the differential expression of fecal metabolites among different groups was determined with t-tests or ANOVA tests using Python; and pathway and network analyses were performed using Mummichog, a set of algorithms specifically designed for high-throughput metabolomics.

All patents, patent applications, provisional applications, and publications referred to or cited herein are incorporated by reference in their entirety, including all figures and tables, to the extent they are not inconsistent with the explicit teachings of this specification.

Following are examples which illustrate procedures for practicing the invention. These examples should not be construed as limiting. All percentages are by weight and all solvent mixture proportions are by volume unless otherwise noted.

### Example 1—Propionibacterium is Enriched in the Intestinal Microbiota of Breast Milk-Fed Infants

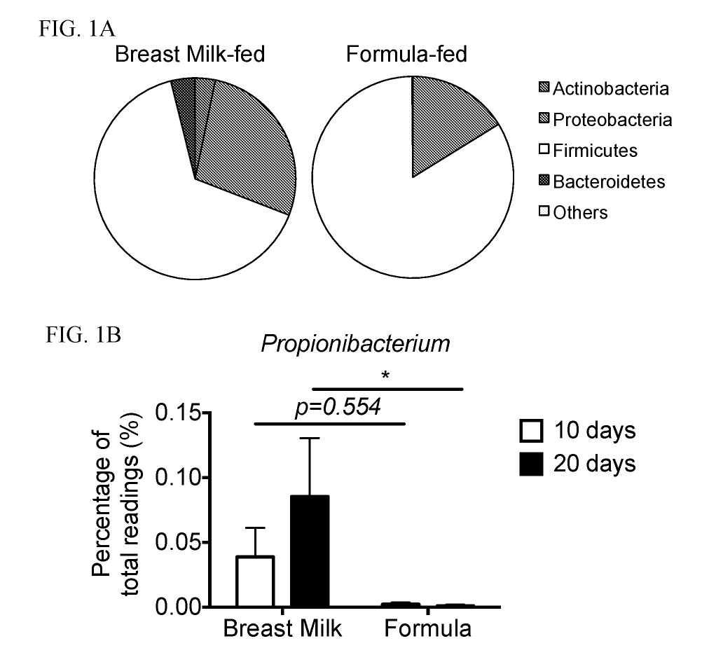

To identify potential probiotic bacterial strains that could be used for the supplementation of pasteurized human donor milk, the intestinal microbiota composition of breast milk-fed and formula-fed infants were investigated. Propionibacterium was found to be enriched in breast milk-fed infants when compared to formula fed-infants (FIG. 1). Therefore, a probiotic bacterial strain belonging to this Propionibacterium, for example, P. freudenreichii, can be used to supplement human donor milk.

### Example 2—P. Freudenreichii Induces Regulatory and Protective Antigen-Dependent Immune Responses in Healthy Hosts

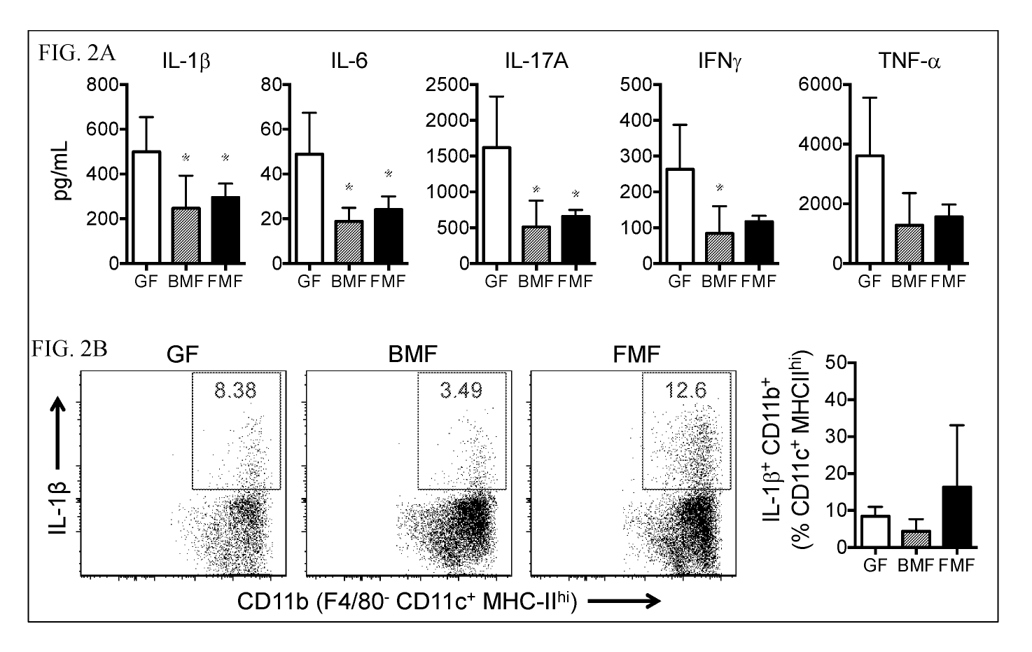

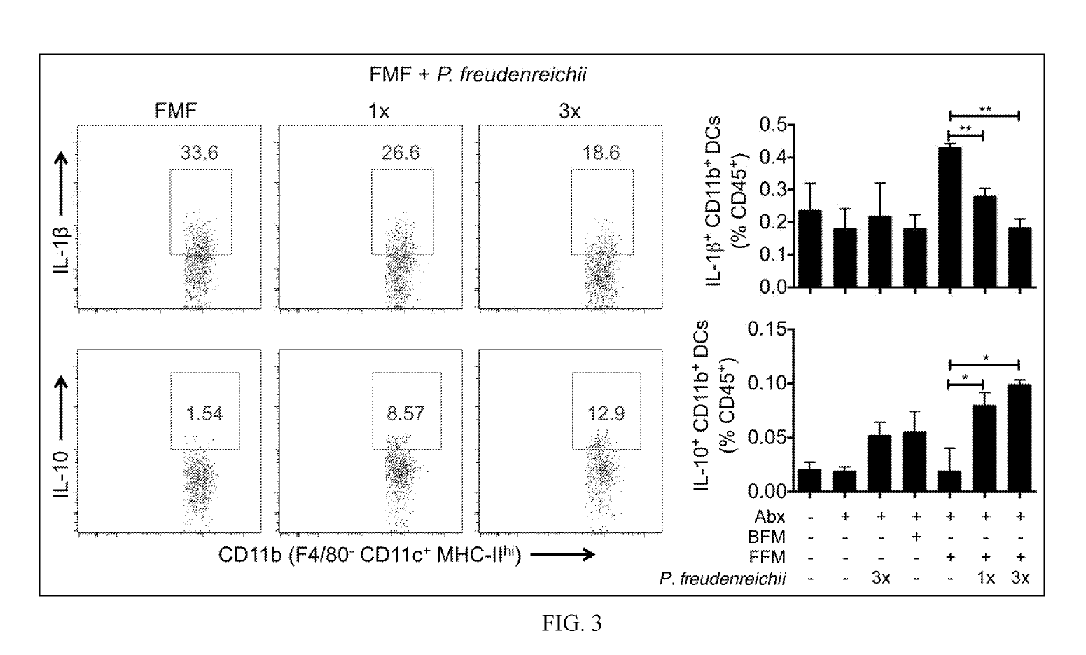

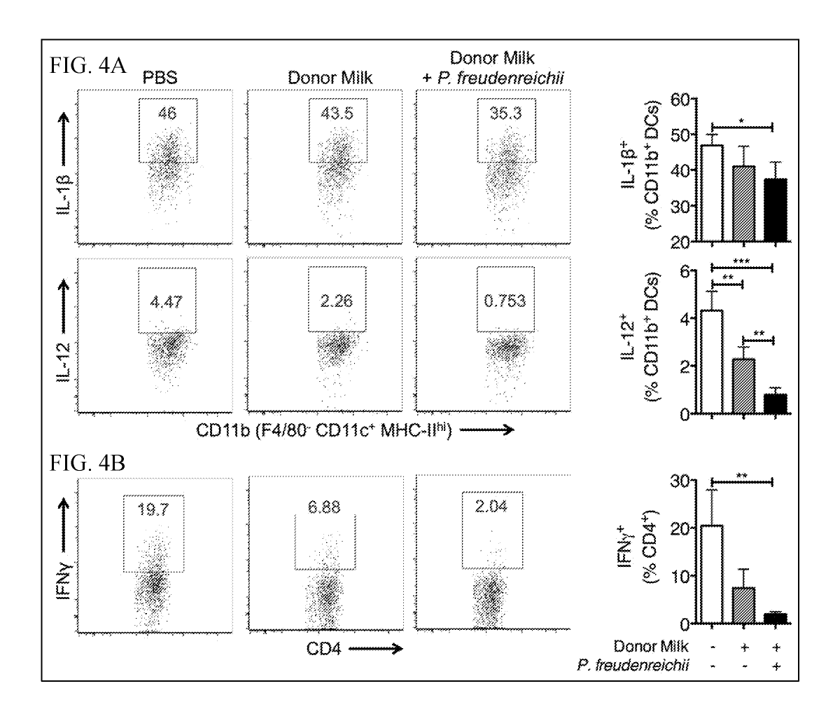

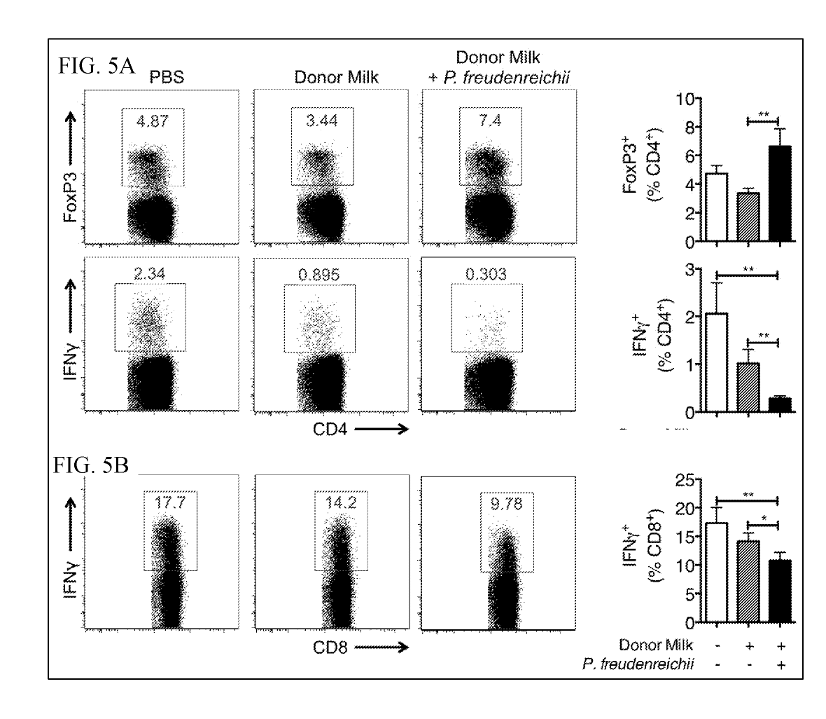

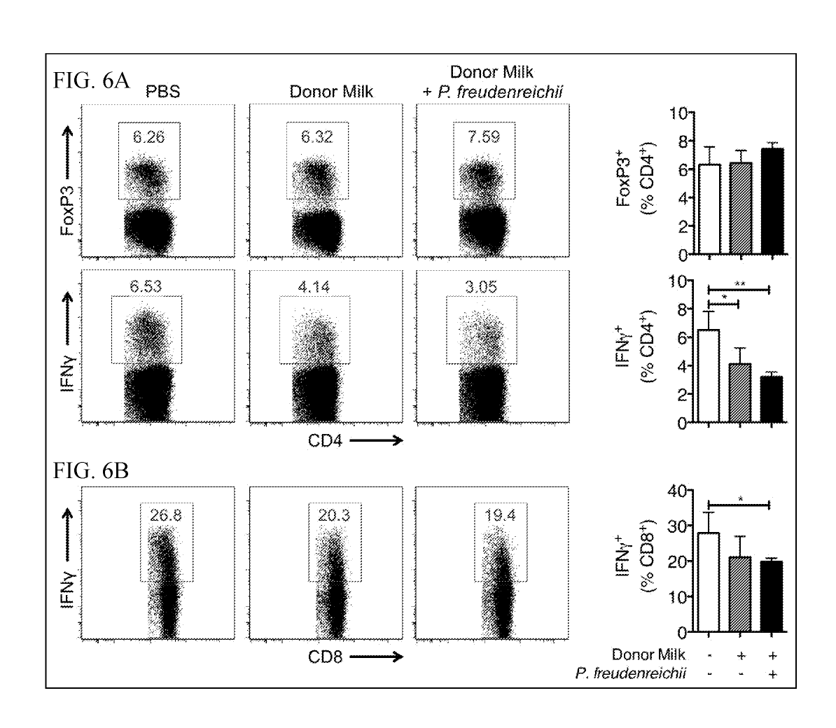

The immune responses induced by P. freudenreichii in steady-state using different mouse models and strains were investigated. GF C57BL/6 (B6) mice were transplanted with microbiota derived from fecal samples from breast milk-fed or formula-fed infants. Only GF mice transplanted with breast milk-fed microbiota demonstrated reduced levels of IL-1β, a pro-inflammatory cytokine, in colonic dendritic cells (DCs) (FIG. 2B). To test whether P. freudenreichii supplementation could recapitulate the results observed with breast milk-fed microbiota transplantation in formula-fed microbiota transplanted mice, conventional B6 mice were depleted of their commensal microbiota with antibiotic treatment. Oral gavage with 10⁹ colony-forming units (CFU) of P. freudenreichii was shown to decrease the frequency of IL-1β⁺ DCs in these mice, while also increasing the number of IL-10⁺ DCs (FIG. 3). IL-1β is considered to be proinflammatory and IL-10 a regulatory cytokine. Subsequently, the beneficial effects of supplementation of human donor milk with P. freudenreichii over feeding with human donor milk alone was tested. Although indications of immune regulation were observed with donor milk alone, supplementation with 10⁹ CFU of P. freudenreichii promoted immune regulation in the colons (lower IL-1β and IL-12 in DCs, and reduced IFNγ in T cells), in the MLNs (increased regulatory T cells (Tregs) and reduced IFNγ in T cells), and in the spleens (reduced IFNγ in T cells) of P. freudenreichii-treated mice (FIGS. 4-6). Nonetheless, it is sometimes beneficial for protective immune responses, which could help clear potential invading pathogens, to be induced in the host.

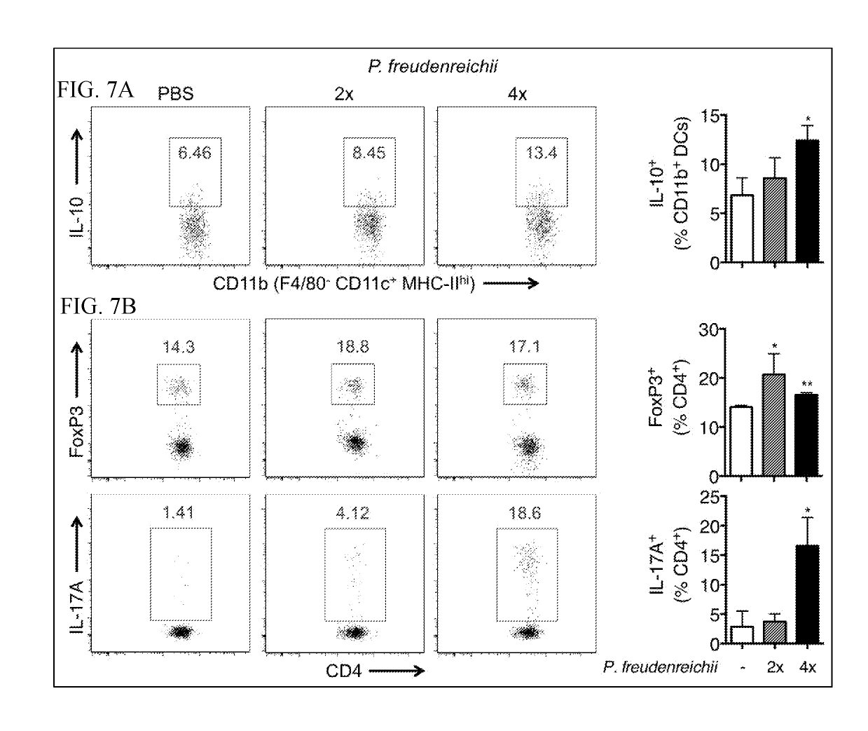

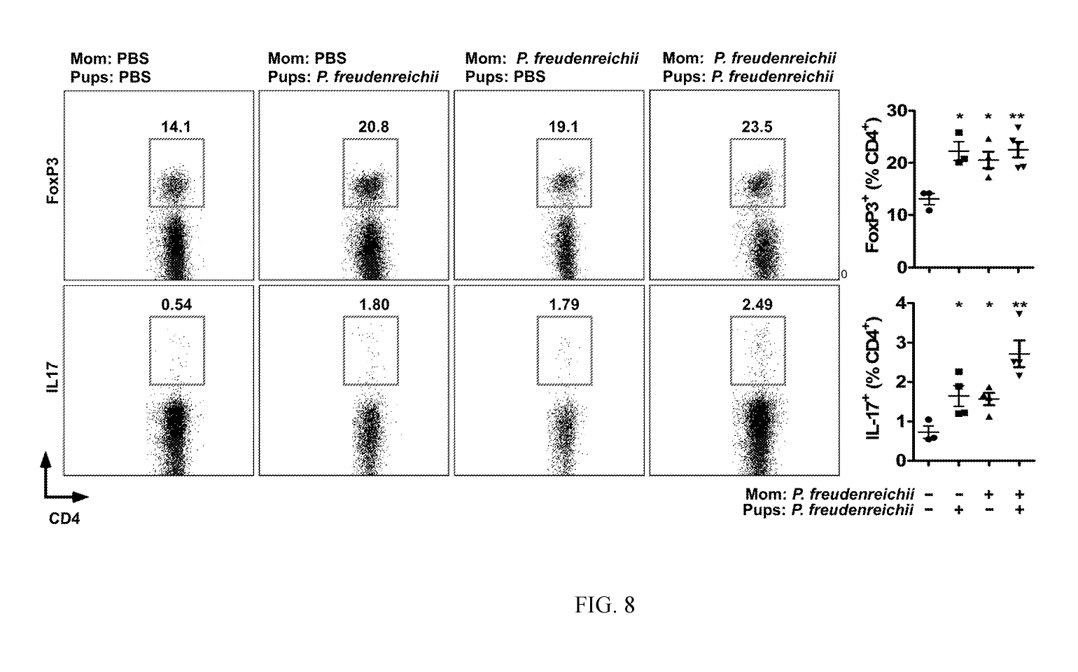

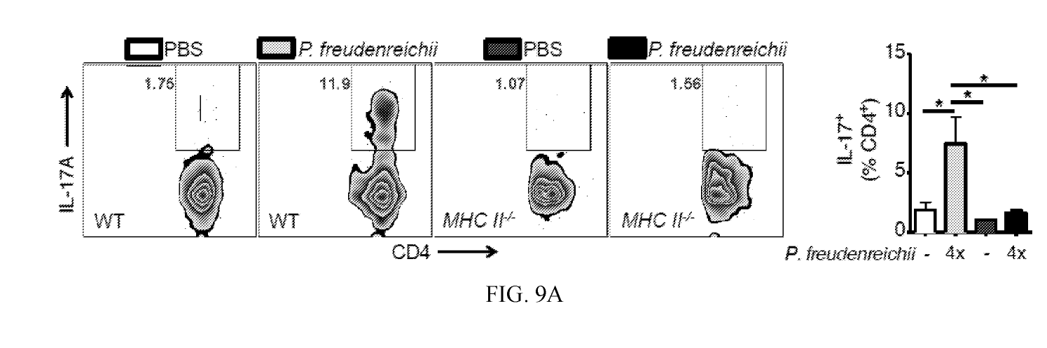

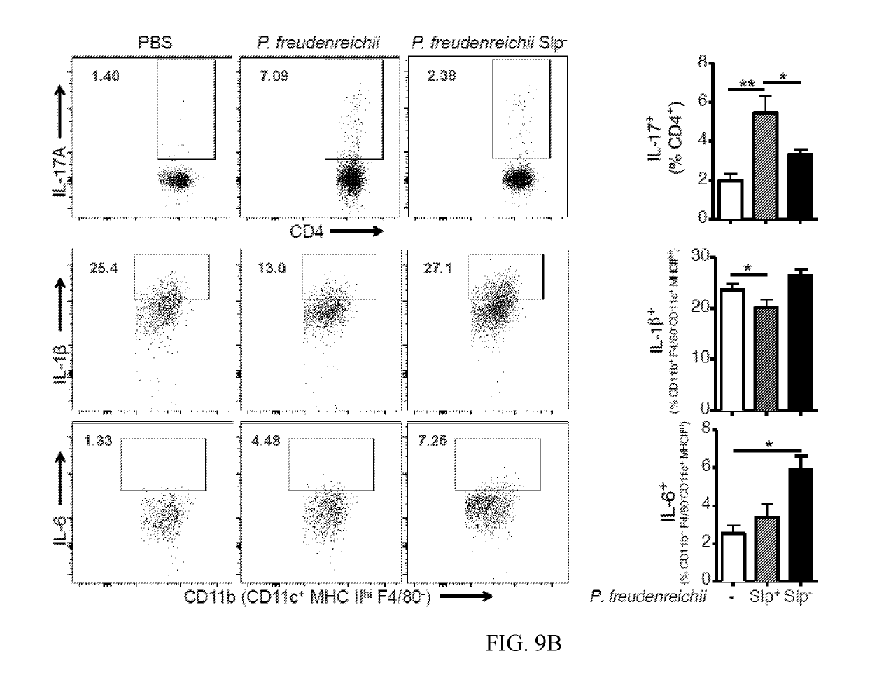

Whether multiple gavages with P. freudenreichii would not induce sustained immune suppression and thus render the host unprotected was also checked. Healthy B6 mice were treated four times with 10⁹ CFU of P. freudenreichii and immune responses were investigated. In addition to increased IL-10⁺ DCs and FoxP3⁺ Tregs, multiple gavages with P. freudenreichii also induced IL-17⁺ T cells (FIG. 7). Th17 responses, characterized by IL-17 production, are very important for pathogen clearance. However, if left unchecked, these responses could result in pathogenic inflammation. Thus, it is especially important that well-functioning Tregs are also present to maintain a healthy immunologic balance that is both regulatory and protective. As such, it was also demonstrated that regulatory and protective Th17 responses were induced in mice that received P. freudenreichii from their mother's milk or starting at a very young age, with the best results obtained when P. freudenreichii feeding continued after weaning (FIG. 8). This Th17 cell formation was significantly abrogated in histocompatibility 2, class II (MHC-II) antigen, beta 1 knock-out mice (H2-Ab1^{-/-} mice), suggesting that these Th17 cells exhibited S-layer dependency (FIG. 9), as DCs deficient in MHC-II are unable to present P. freudenreichii antigens to T cells and promote protective Th17 immunity.

### Example 3—P. Freudenreichii is Protective in Experimental Models of Colitis

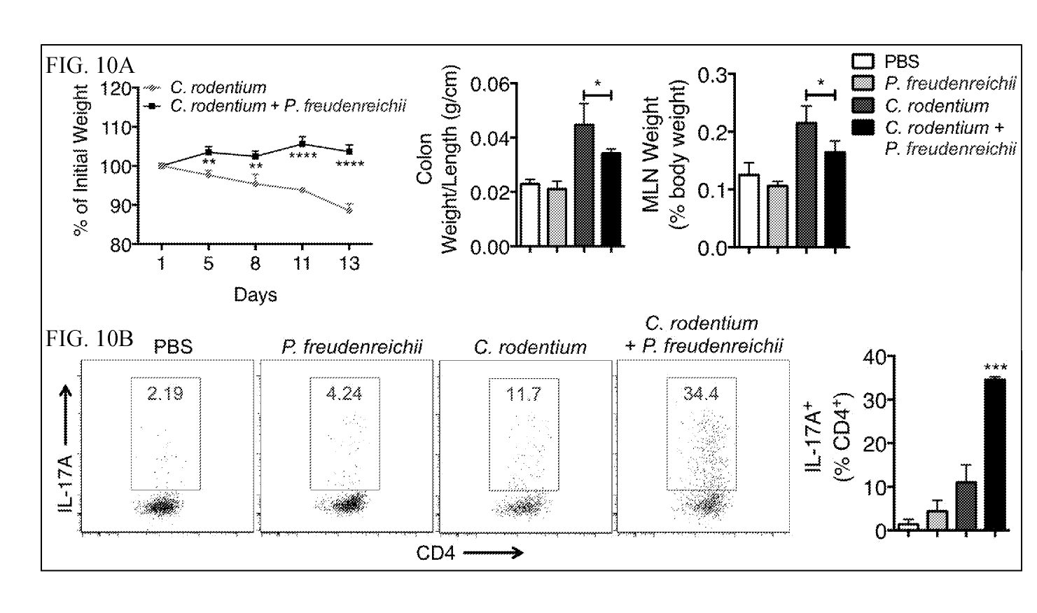

Protective effects of P. freudenreichii in an infectious model of colitis were examined. B6 mice were infected with 10⁹ CFU of Citrobacter rodentium, and either left untreated or treated with 10⁹ CFU of P. freudenreichii at the specified time-points (FIG. 10). Protection from colitis in this model would require immune responses that could help clear the infection while also dampening the destructive inflammation induced by the pathogen itself. P. freudenreichii-treated mice were better protected from C. rodentium-induced colitis as measured by reduced weight loss and decreased intestinal inflammation (FIG. 10A). Moreover, higher Th17 immunity was observed in infected mice treated with P. freudenreichii (FIG. 10B), which may have contributed to the protection observed.

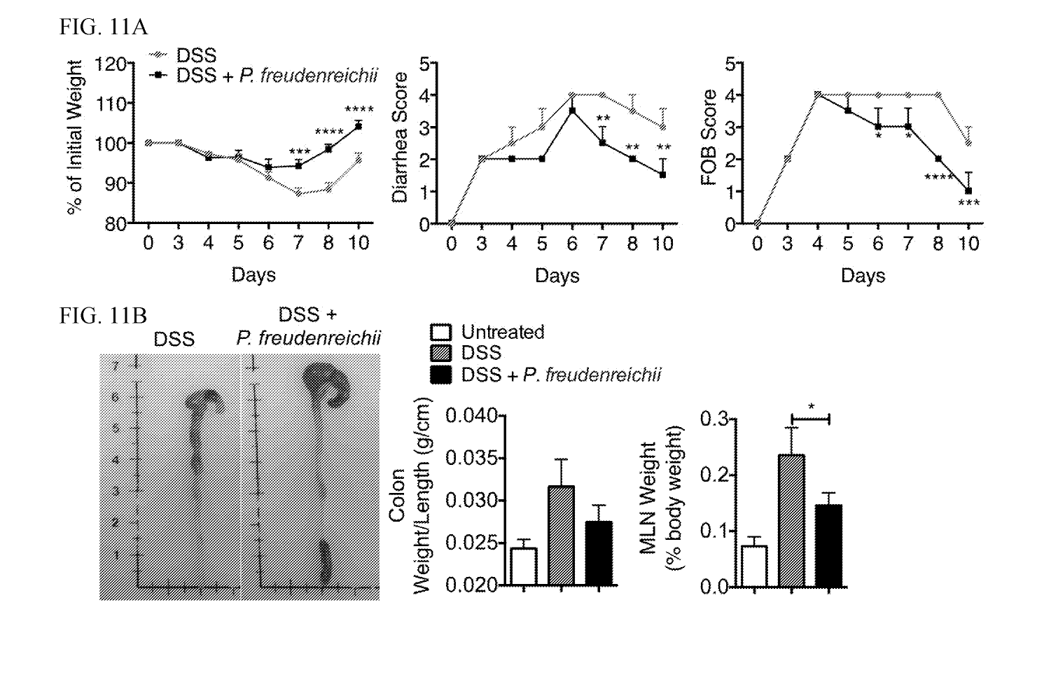

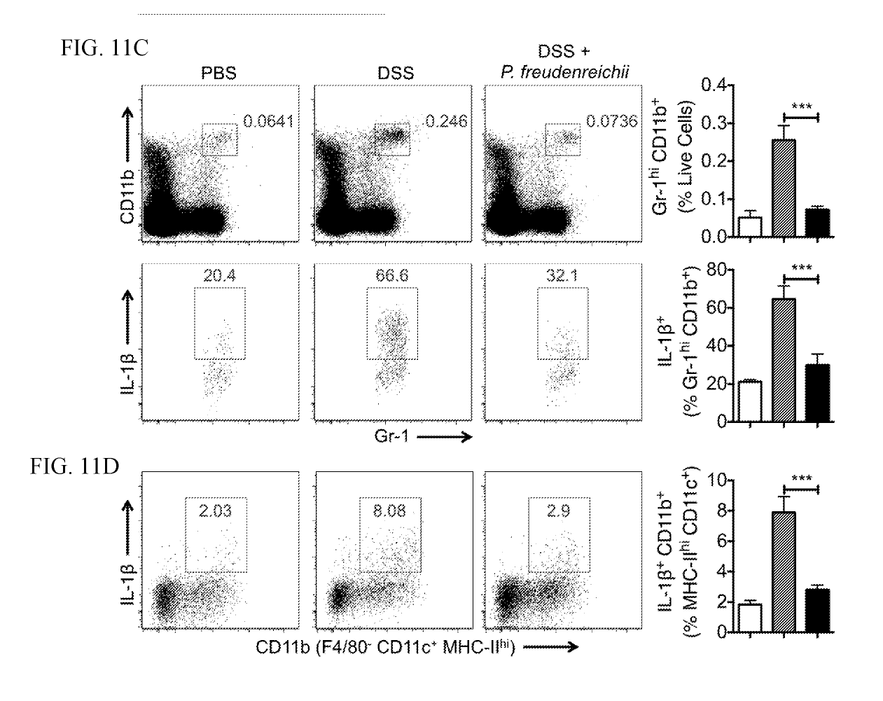

Additionally, another murine model of colitis whereby intestinal damage is induced with dextran sulfate sodium (DSS) was tested. Mice were given 3% DSS in the drinking water for 5 days. Mice treated with P. freudenreichii lost less weight and had improved diarrhea scores with lower fecal occult blood (FOB) scores (FIG. 11A). P. freudenreichii treatment also reduced gross colonic inflammation (FIG. 11B), and decreased immune infiltration and activation in the MLNs (FIGS. 11C and 11D).

### Example 4—Mechanism of Bacterial-Dependent Protective Response Mediated by P. Freudenreichii

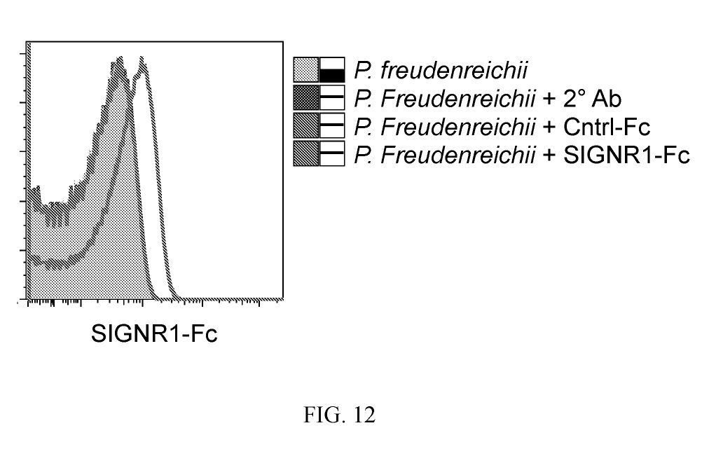

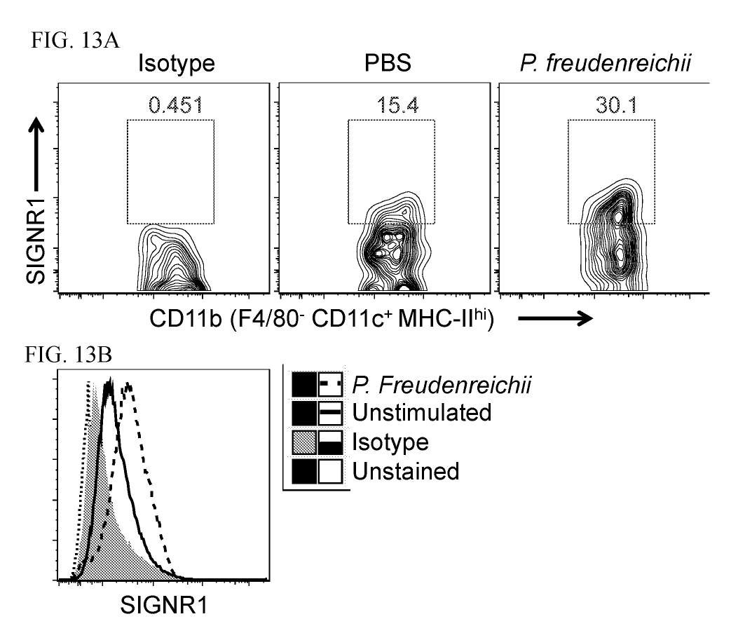

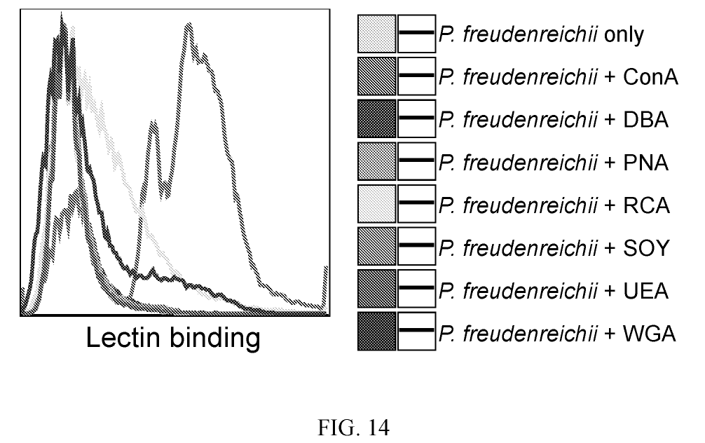

Propionibacterium metabolically produces propionic acid, all 20 amino acids, and many vitamins (e.g., vitamin B₁₂), all of which play vital roles in intestinal homeostasis, metabolism, and regulated immune protection. P. freudenreichii was found to bind to C-type lectin specific intercellular adhesion molecule-3-grabbing nonintegrin-related 1 (SIGNR1) and this in turn may regulate receptor-specific endocytosis of P. freudenreichii S-layer protein for antigen processing and presentation (FIG. 12). This C-type lectin is constitutively expressed by colonic DCs and can be upregulated in P. freudenreichii-treated mice (FIG. 13A) and in vitro (FIG. 13B). SIGNR1 may internalize P. freundenreichii S-layer in an MHC-II restricted manner in innate cells (e.g., DCs) to polarize T cells toward antigen-specific Th17 responses, inducing protective immunity against gastrointestinal microbial infection. P. freudenreichii S-layer consists of a polysaccharide characterized by galactose, mannose and rhamnose, causing this bacterium to bind selectively to lectins, including concanavalin A (ConA) (FIG. 14).

As such, the invention provides the connection between a beneficial gut microbe, its metabolites, and the host cells expressing critical sensing receptor such as SIGNR1 to initiate and control protective intestinal immunity. Development of less costly and non-invasive therapies for pathogenic inflammation-induced intestinal disorders, by providing human milk supplemented with this beneficial bacterium to newborn infants, are also provided.

It should be understood that the examples and embodiments described herein are for illustrative purposes only and that various modifications or changes in light thereof will be suggested to persons skilled in the art and are to be included within the spirit and purview of this application and the scope of the appended claims. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

Isolation of P. freundenreichii from the Infant Feces

The feces from breast fed babies obtained from the repository were used to isolate the bacteria Propionibacterium freundenreichii. 100 milligrams of feces was dissolved in sterile lectin-binding buffer (PBS, 20 mM CaCl2 and 20 mM MgCl2), and 100 micrograms of biotinylated Concanavalin A (ConA) was added to the suspension. The contents were incubated at 4° C. along with mild shaking for three hours. The fecal contents were then washed with the lectin-binding buffer three times. The Streptavidin-ferrous beads were introduced to the washed fecal contents to allow the ConA bound bacteria to bind to the beads. The ferrous beads were then washed six times with lectin-binding buffer in the presence of a magnet. The washed contents were spread over several MRS-lactate agar plates in order to grow P. freundenreichii. Several colonies were obtained from the plate after three days. DNA were subsequently isolated from these individual colonies, and their identity were determined by the amplification of 16sRNA and its sequencing. We designated this isolate of the bacteria Propionibacteria freundenreichii UF-1 (P.UF-1).

First Draft of the Genome Sequence Analysis of P. freundenreichii

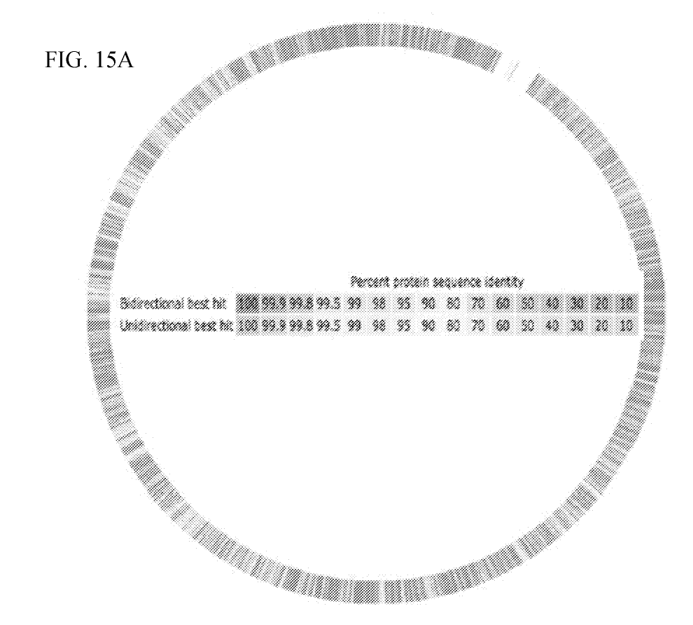

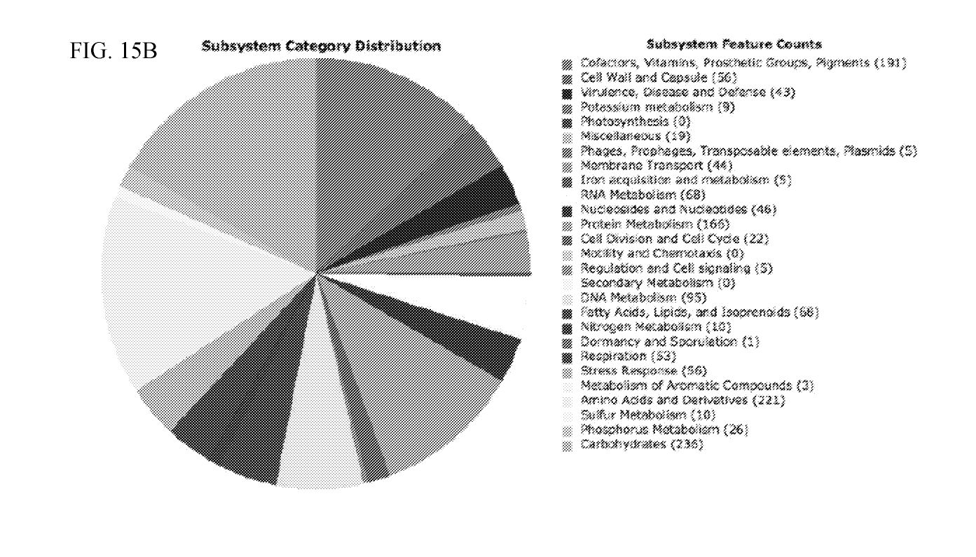

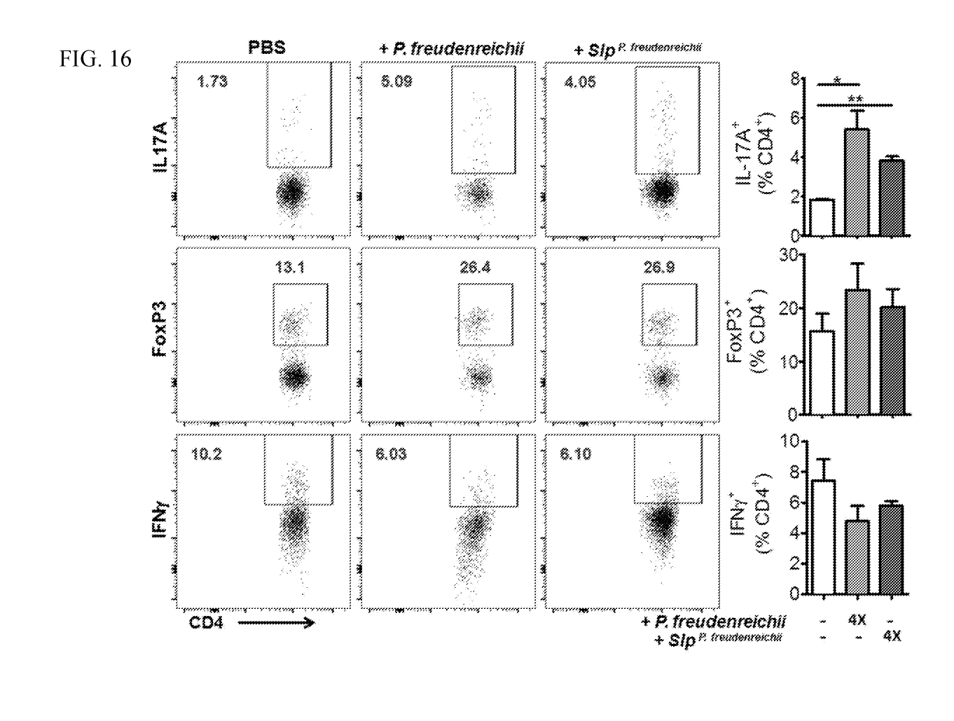

To characterize the genome of the Propionibacterium freundenreichii, DNA was isolated and then sequenced on the Pacific Bio platform at ICBR. The genome was assembled on the RAST platform and compared with the known sequence of Propionibacterium to evaluate the similarity and dissimilarity between the two sequences (FIGS. 15A and 15B).

Protective Effect of P. freudenreichii is Located on its Surface Layer Proteins

Since, an antigen dependent increase in Th17 cells in the colon was observed upon treatment with P.UF-1, we sought to elucidate whether these antigens are present on the surface of the bacteria, and if the surface proteins are capable of inducing such a response alone, without the presence of the bacteria. To test this hypothesis we gavaged mice either with P.UF-1 or isolated surface proteins from P.UF-1, and the subsequent induction of Th1, Th17, and Tregs were compared against PBS treated mice. The data clearly demonstrated that the mice groups treated with intact bacteria or those surface proteins isolated from the bacteria showed an significant increase in Th17 cells within the gut, which is essential for the protective effects of P.UF-1 to be observed (FIG. 16). This data also raises the possibility of utilizing a bacteria free therapeutic and/or preventive approach for addressing the needs of colitis prone individuals.

### Example 5—Induction of dlaT-Specific Th17 Cell Differentiation by the P. UF1 Bacterium and its Dependency on Signr1

NEC, an intractable cause of mortality in preterm infants, is attenuated by feeding human breast milk (H-BM). This attenuation correlates with alterations in the gut microbiota, particularly enrichment of Propionibacterium species. Transfaunation of microbiota from HBM-fed preterm infants, or a Propionibacterium strain, P. UF1, cultured therefrom, to mice conferred protection against pathogens and correlated with profoundly increased intestinal Th17 cells, sustained functional regulatory T cells, and reduced inflammasome-associated IL-1β. Metabolomic studies, followed by use of genetically engineered mice and bacteria revealed that immunomodulation by P. UF1 was mediated by bacterial dihydrolipoamide acyltransferase, resulting in SIGNR1 activation on intestinal phagocytes. Together, these results mechanistically elucidate the protective effects of HBM and P. UF1-induced immunoregulation that safeguard against inflammatory diseases, including NEC.

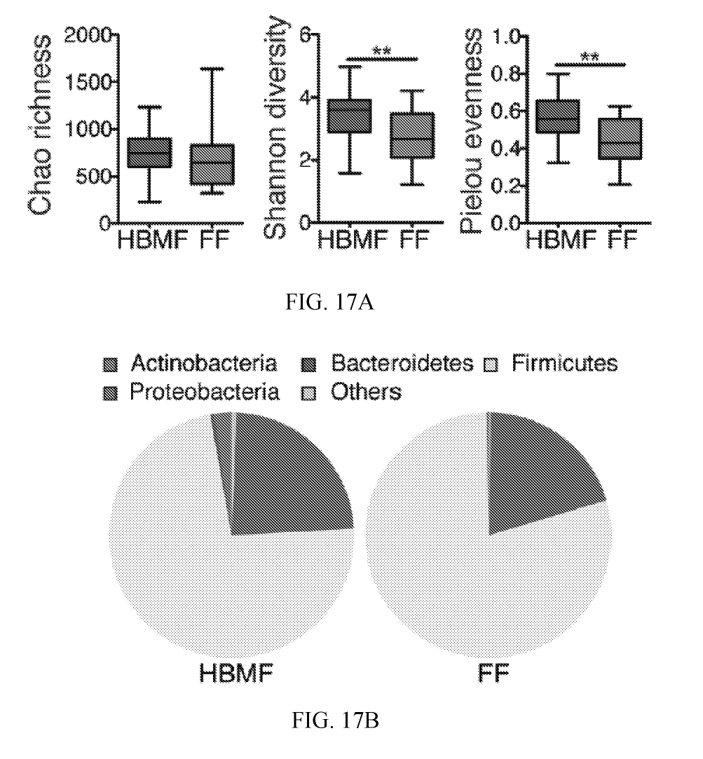

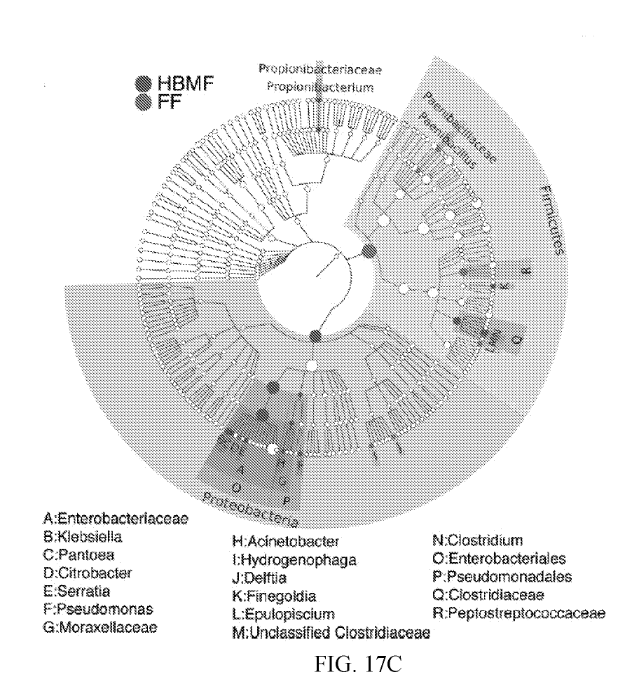

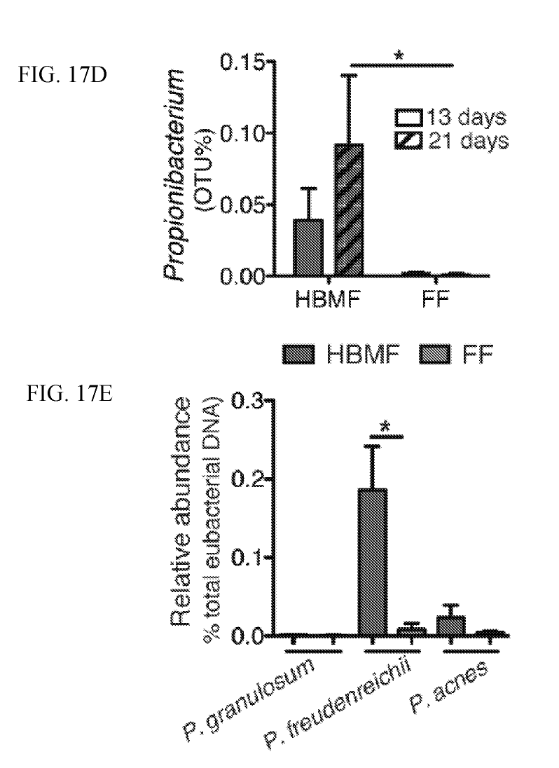

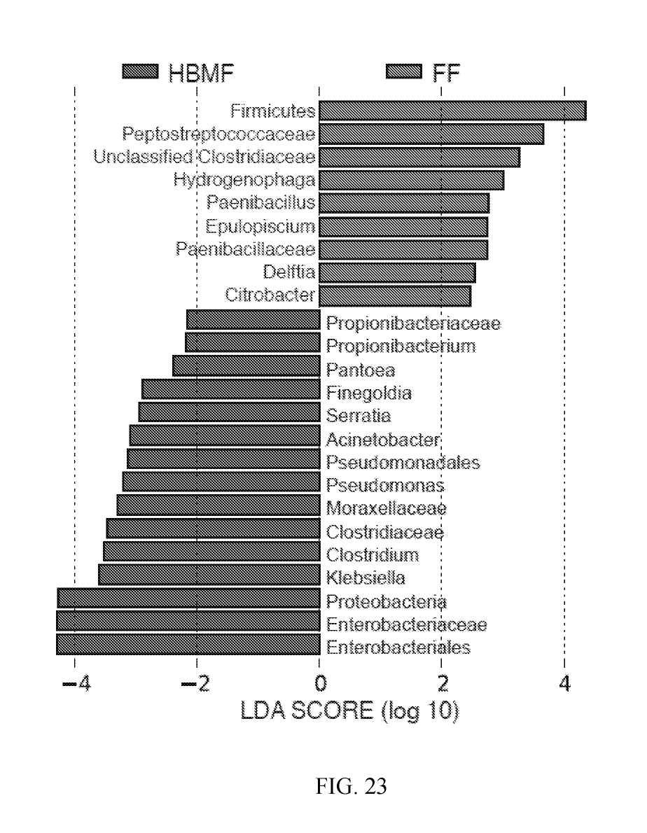

The composition and diversity of the gut microbiota of human breast milk-fed (HBMF) preterm infants (n=20) is distinct from that of formula-fed (FF) preterm infants (n=20). Microbiota analyses of these two cohorts revealed major differences in the Shannon diversity and the Pielou evenness index (p<0.01) (FIG. 17A and FIG. 23). Moreover, at 21 days after birth, microbiota compositions between the HBMF and FF preterm infants were principally different in the Actinobacteria phylum, among other bacteria (FIG. 17B). Thus, the focus was drawn to key species of this bacterial phylum, particularly Propionibacterium, which heavily contributed to these differences (FIGS. 17C-17D). Importantly, the proportion of the Propionibacterium genus, including P. freudenreichii, in the microbiota of the HBMF preterm infants was significantly increased throughout days 13 to 21 after birth, while these bacteria were poorly detected in the feces of the FF preterm infants (FIG. 17E). Using specific bacteria culturing broth and a carbohydrate separation approach, Propionibacterium strains were isolated from the feces of HBMF preterm infants for their characterization.

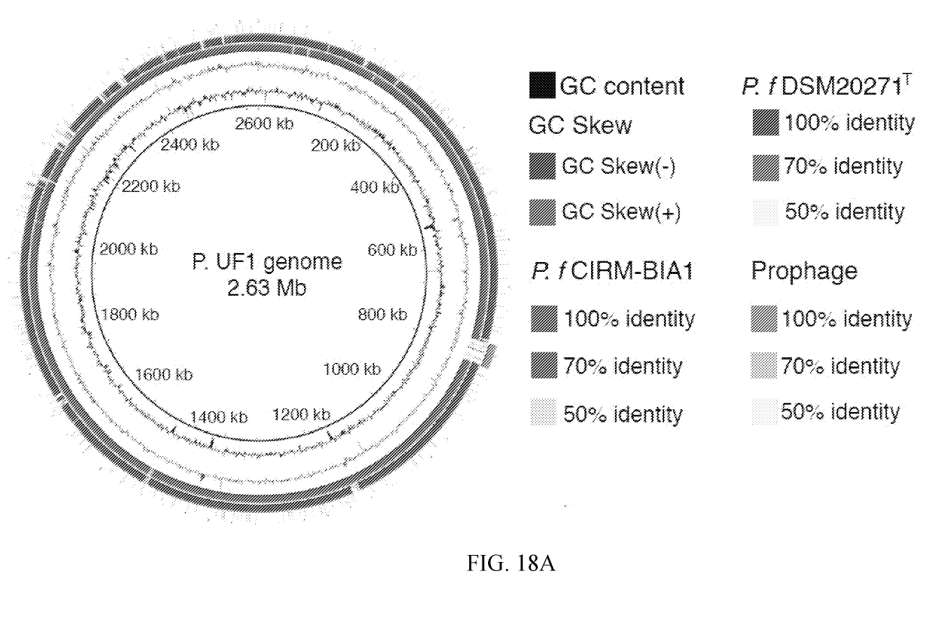

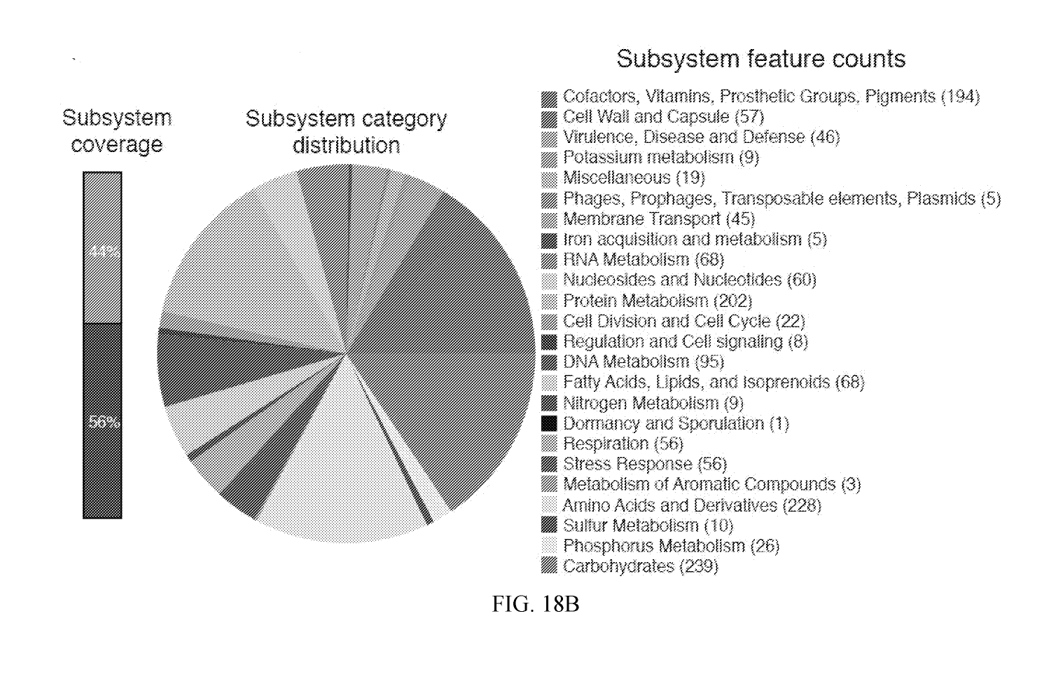

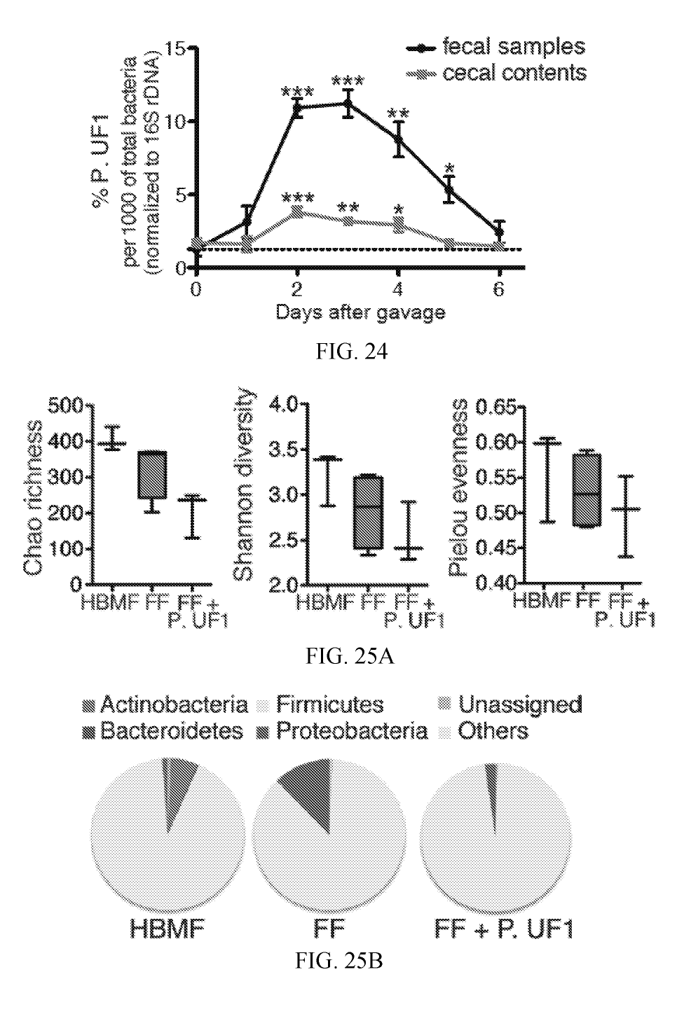

The first draft genome-sequence analyses of one of these newly identified Propionibacterium species, designated, P. UF1, exhibited only 90% identity to known Propionibacterium species, including P. freudenreichii subsp. freudenreichii DSM20271^{T} and subsp. shermanii CIRM-BIA1 (FIG. 18A). The genome of P. UF1 (2.63 Mb) encodes for critical enzymes involved in the bacterial respiratory chain, fermentation, catabolism, and biosynthetic pathways for all amino acids (FIG. 18B). Oral gavaging of C57BL/6 mice resulted in transient gut colonization with P. UF1, whereby the P. UF1 strain was no longer detectable in the fecal samples or cecal contents of orally treated mice after 5-6 days of its administration (FIG. 24).

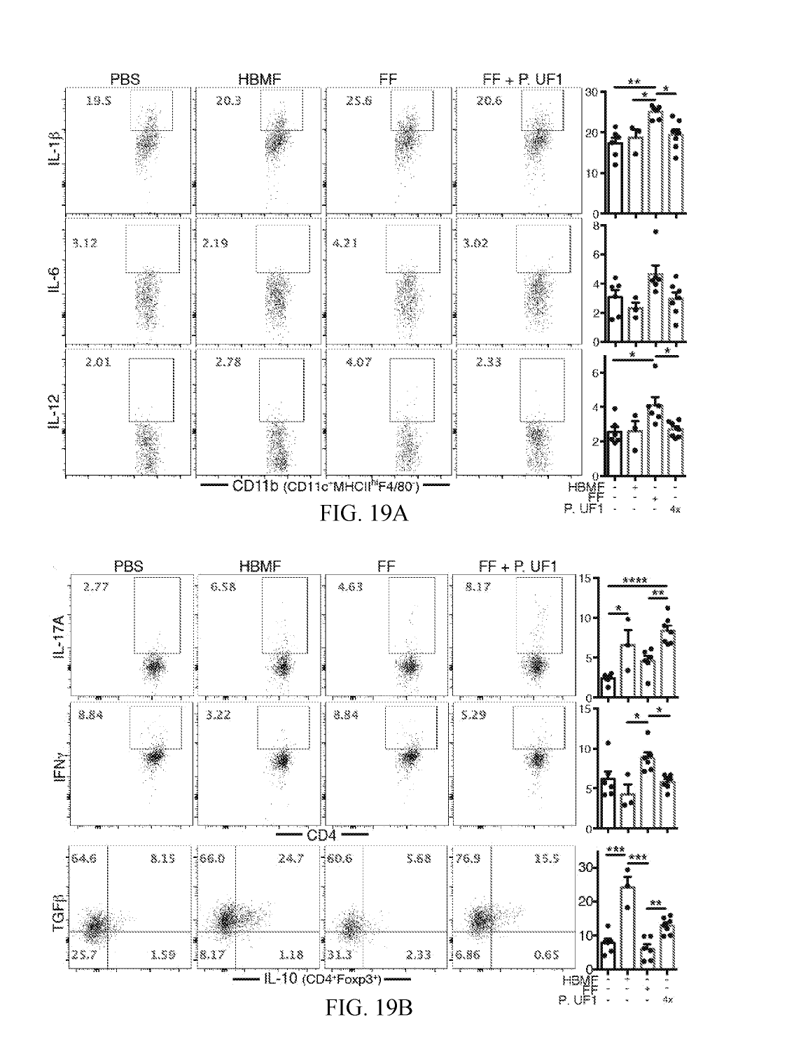

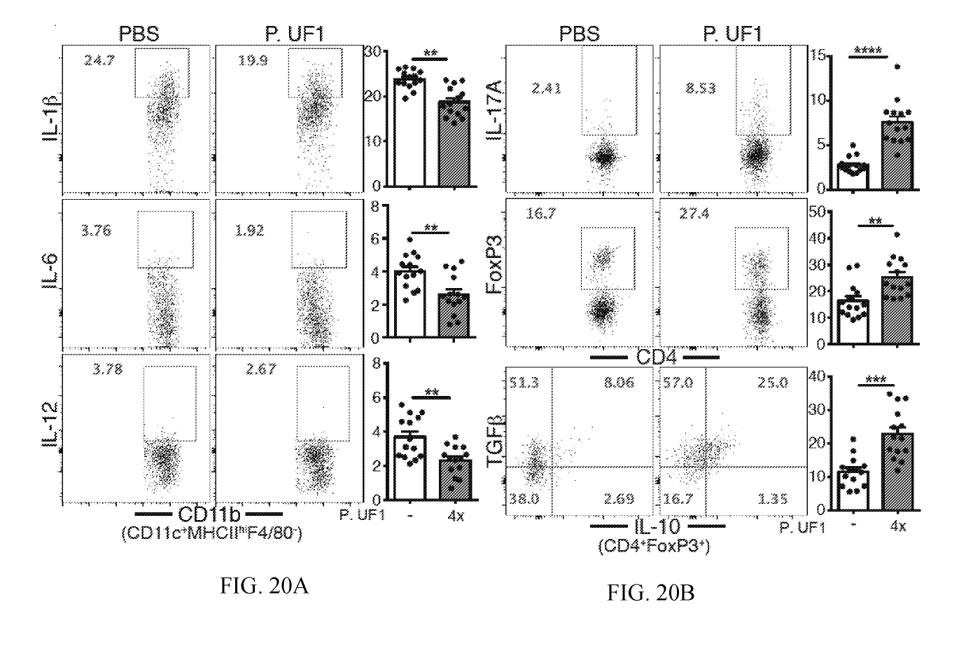

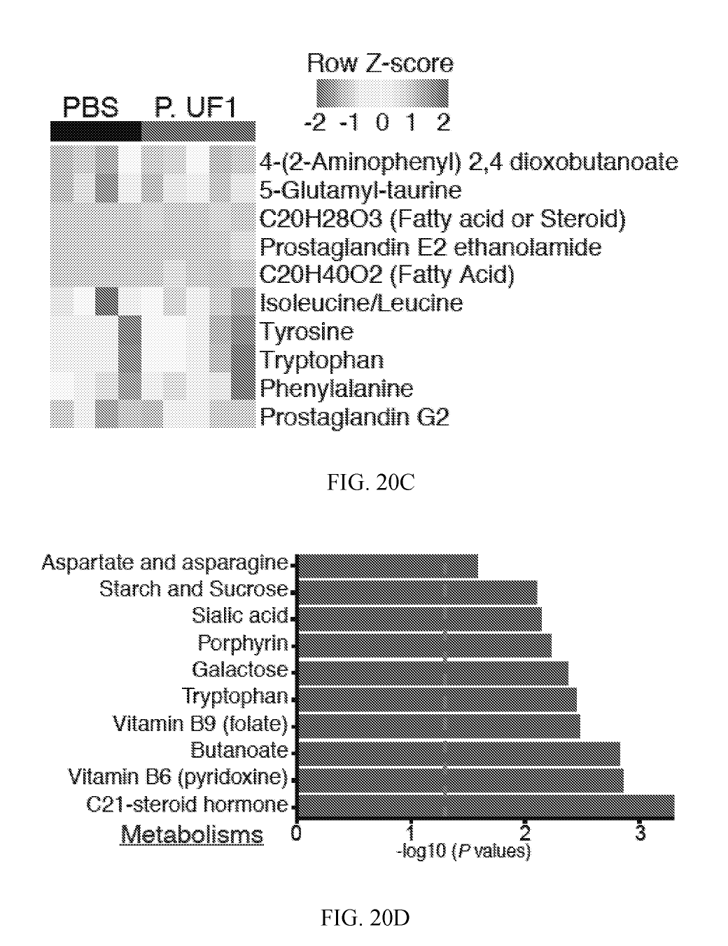

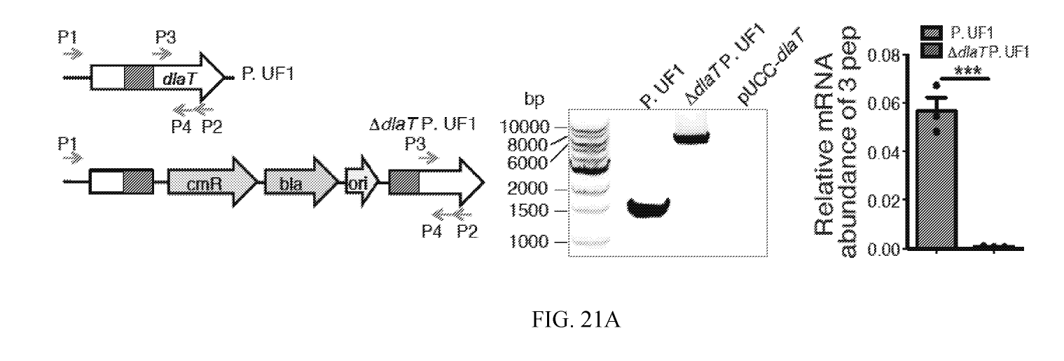

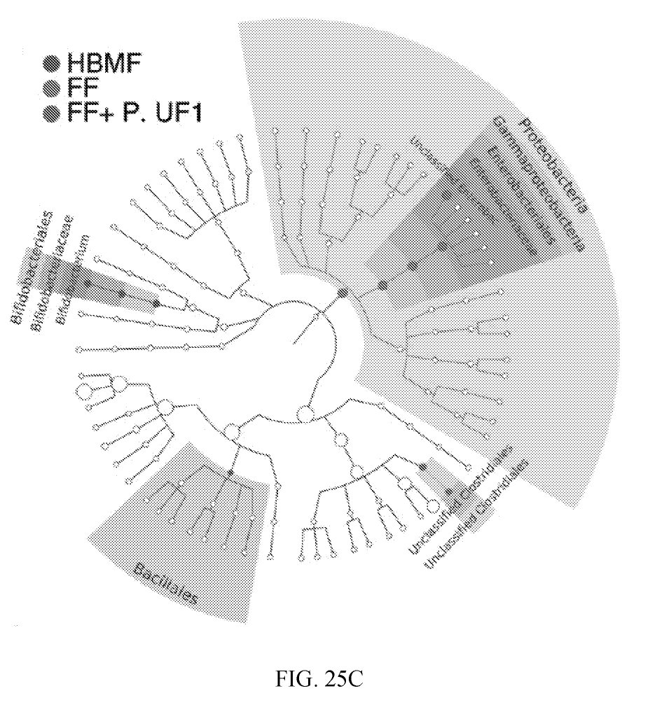

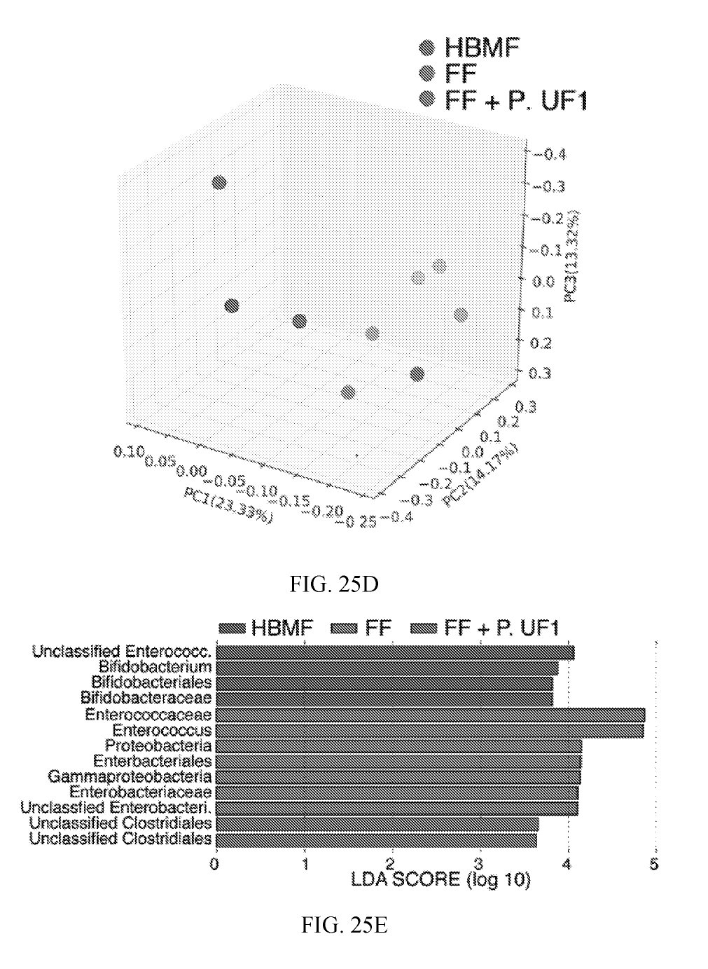

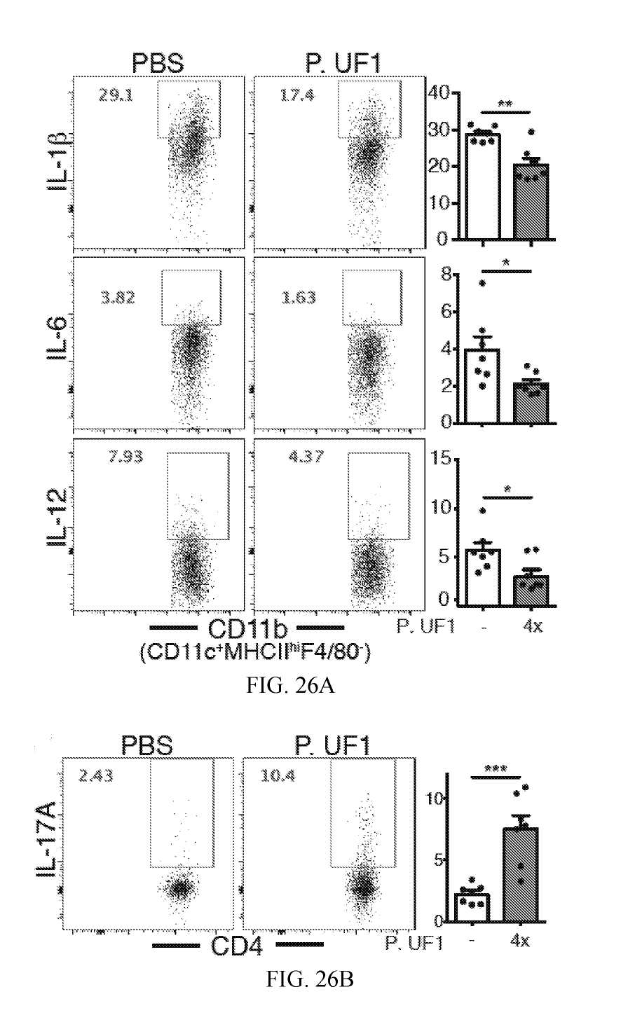

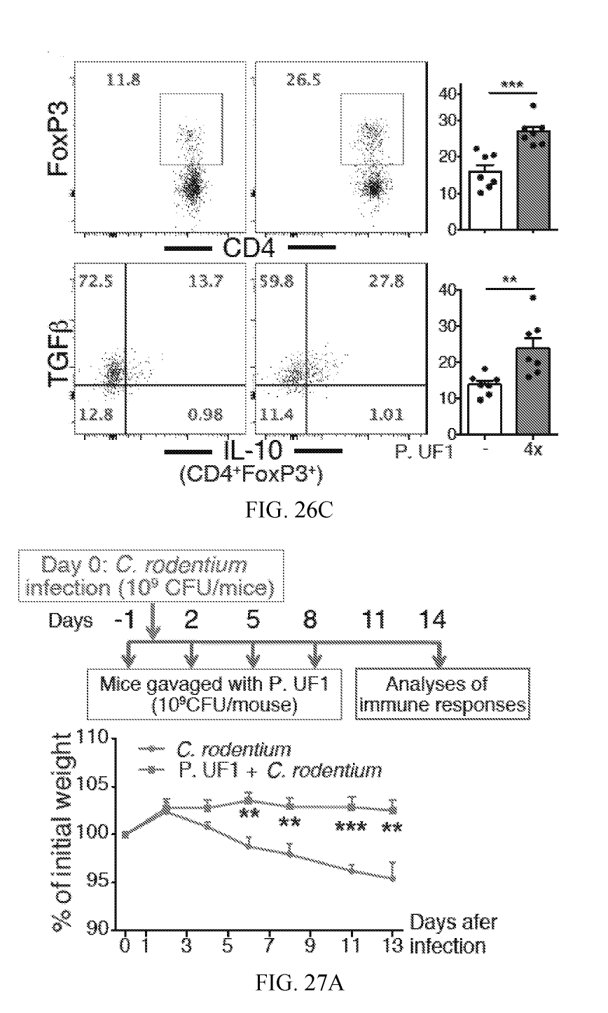

Fecal microbes derived from HBMF and FF preterm infants were transfaunated into C57BL/6 germfree (GF) mice. GF mice receiving the microbiota from HBMF preterm infants did not exhibit the enhanced proinflammation (e.g., IL-1β) in colonic dendritic cells (DCs), which was observed in the GF mice that were transfaunated with microbiota from the feces of FF preterm infants (FIG. 19A). Moreover, transfaunating GF mice with HBMF preterm infants' microbiota resulted in increased frequencies of Th17 cells and sustained function of regulatory T cells (Tregs); a trend that was less evident in GF mice transfaunated with microbiota from FF preterm infants (FIG. 19B). Not only was proinflammatory DC activation diminished upon P. UF1 gavage of GF mice that were transfaunated with microbiota from FF preterm infants, but the frequencies of Th17 cells and Tregs were also significantly increased to the levels seen in GF mice that received microbiota from HBMF preterm infants. Also, IFNγ⁺ Th1 cell responses were decreased (FIGS. 19A-19B). Additionally, the composition of the gut microbiota in GF mice that were transfaunated with FF microbiota and subsequently fed P. UF1 showed changes in bacterial organization, including decreased levels of Proteobacteria when compared to the FF microbial community, indicating the potential influence of P. UF1 on the FF microbiota (FIG. 25). To further evaluate the effect(s) of P. UF1 on colonic and peripheral immunity, naive conventional C57BL/6 mice were gavaged with P. UF1. P. UF1 regulated proinflammation (e.g., IL-1β) in DCs and significantly induced the formation of colonic Th17 cells and Tregs in these mice (FIG. 26). To specifically investigate the P. UF1-induced differentiation of Th17 cells, GF mice were monoassociated with P. UF1, and DC and T cell responses were analyzed two weeks later. The levels of proinflammatory IL-10, IL-6 and IL-12 were significantly downregulated in colonic DCs, while the generation of Th17 cells and Tregs in P. UF1-treated mice was greatly increased when compared to PBS-treated GF mice suggesting that this bacterium not only critically regulates immune responses, but also specifically induces the differentiation of Th17 cells (FIGS. 20A-20B). Furthermore, liquid chromatography-mass spectroscopy (LC-MS) analyses of the induced metabolites in the fecal samples of these GF mice monoassociated with P. UF1 showed increased levels of key metabolites, including tryptophan, tyrosine and phenylalanine (FIG. 20C), which potentially regulate Th17 cells. Pathway analyses also revealed that these enriched gut metabolites (FIG. 20D) are highly involved in several immune regulatory pathways, including C21-steroid hormone biosynthesis, tryptophan metabolism, multiple vitamin biosynthetic pathways (e.g., vitamin B₁₂), and porphyrin biosynthesis.

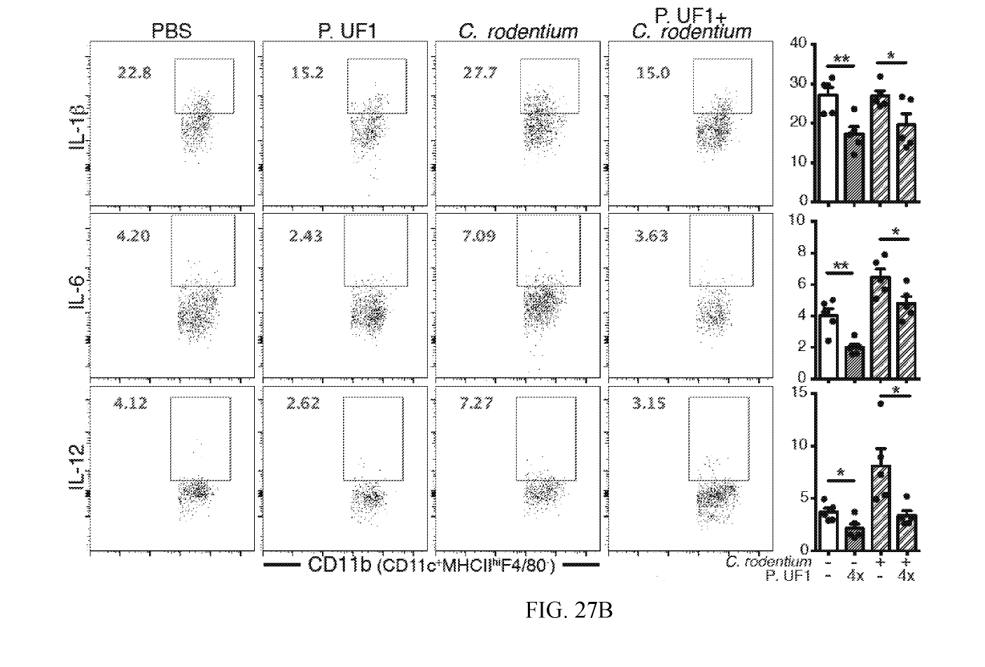

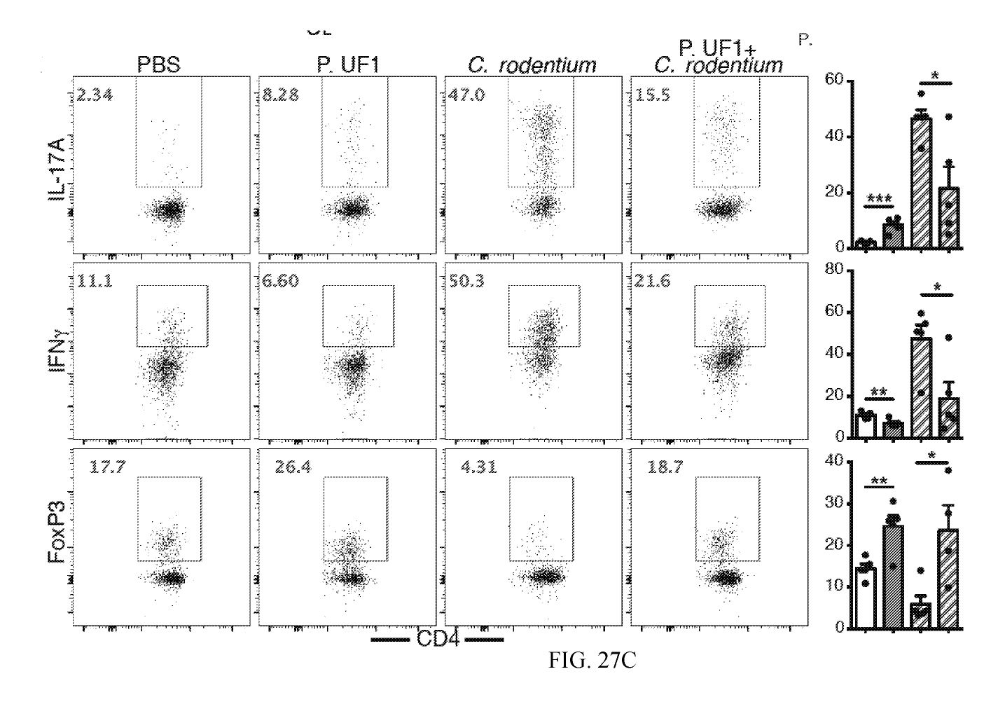

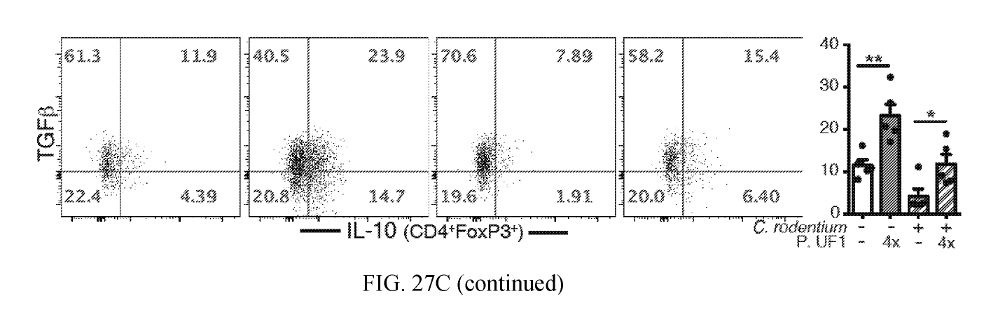

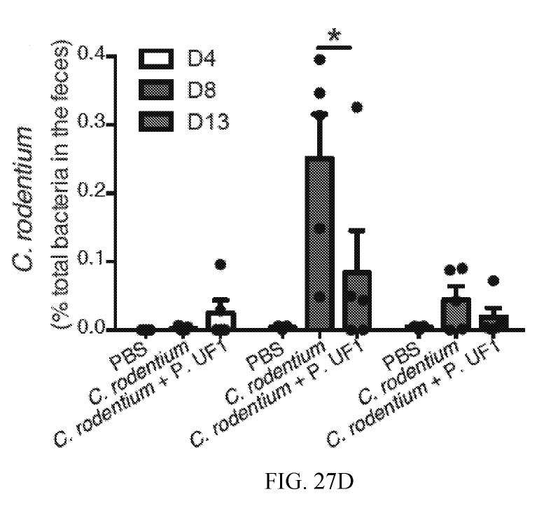

To gain additional information regarding induced immune regulation, particularly Th17 cell responses exerted by P. UF1, the C. rodentium-induced colitis model was employed. In this infectious model, P. UF1 not only significantly mitigated induced inflammation (e.g., IL-1β) that results in weight loss, and regulated Th1 and Th17 cell responses, but also maintained the frequency of induced Tregs upon C. rodentium infection (FIGS. 27A-27C). This resulted in rapid resolution of C. rodentium infection in these mice when compared to C. rodentium-infected mice without treatment, highlighting the regulatory potency of P. UF1 upon pathogen infection (FIG. 27D).

### Example 6—the Role of Surface Layer Proteins in Immune Response

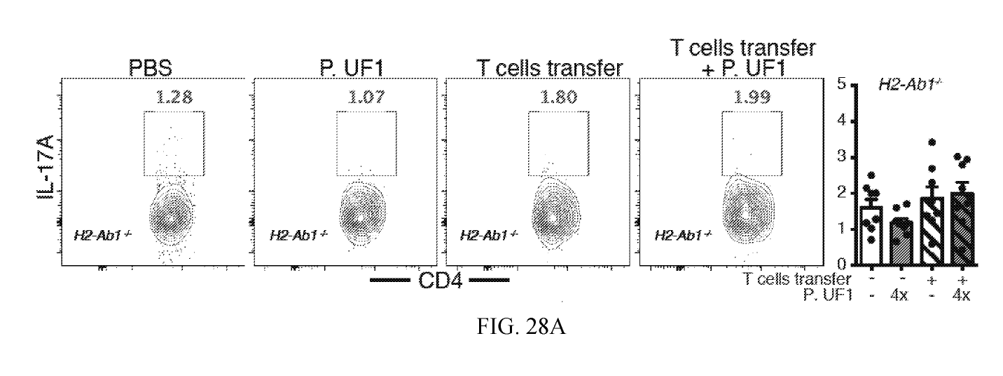

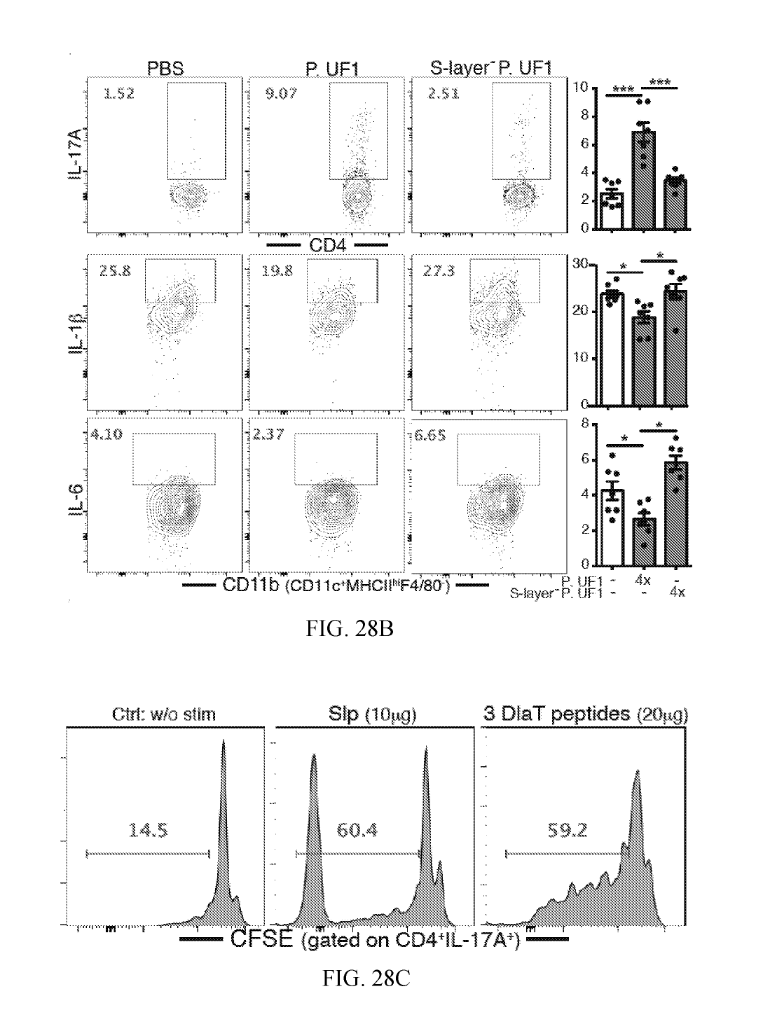

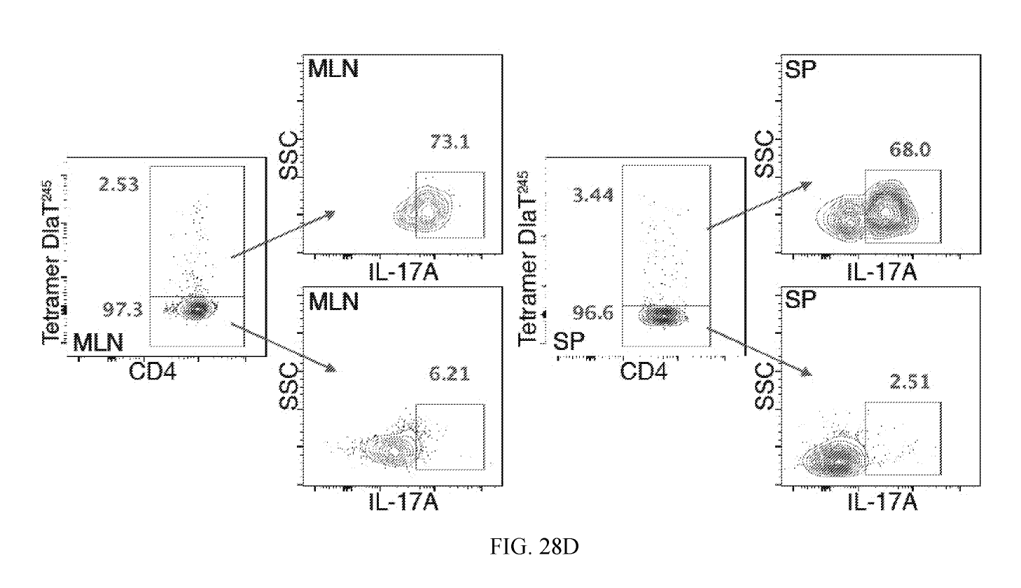

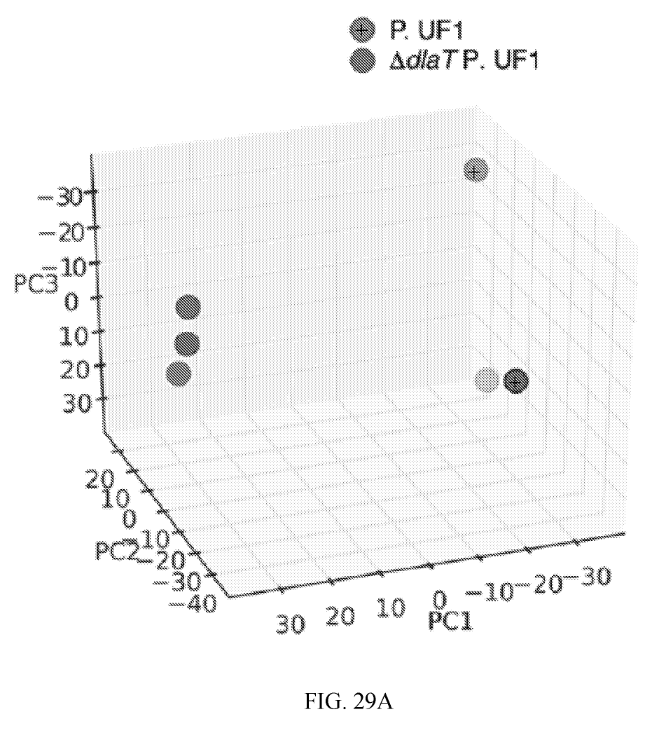

To investigate the potential role of the P. UF1 S-layer in Th17 cell differentiation, this bacterial S-layer was isolated by guanidinium hydrochloride (GdnHCl) and characterized by mass spectrometry. Dihydrolipoamide acyltransferase (DlaT) is the major protein in the P. UF1 S-layer fraction. Three 15-mer peptides from DlaT that exhibit high affinity binding to MHC II were deduced. DlaT is involved in pyruvate decarboxylation that links glycolysis to the citric acid cycle; however, this protein could also be expressed by other, yet to be identified mechanism(s) on the bacterial S-layer to potentially exert immune activation. P. UF1 S-layer-induced Th17 cells depend on the S-layer's presentation via MHC II, as deficiency of MHC II in H2-Ab1^{-/-} mice resulted in the ablation of Th17 cell formation (FIG. 28A). The differentiation of these Th17 cells was not strictly cytokine dependent (e.g., IL-1β, IL-6), since no Th17 cell expansion was observed, even though these cytokines were significantly induced in mice that were orally treated with the P. UF1 whose S-layer was deleted by GdnHCl (S-layer P. UF1), denoting that the S-layer is essential in Th17 cell differentiation (FIG. 28B). C57BL/6 mice were orally gavaged with P. UF1 and splenic CD4⁺ T cells were sorted and co-cultured with bone marrow-derived DCs pulsed with S-layer proteins or with the selected three DlaT peptides. Generation of DlaT-specific Th17 cell proliferation was observed, whose specificity was then confirmed by DlaT-specific tetramer²⁴⁵ for Th17 cells isolated from mice that were fed P. UF1 (FIGS. 28C-28D).

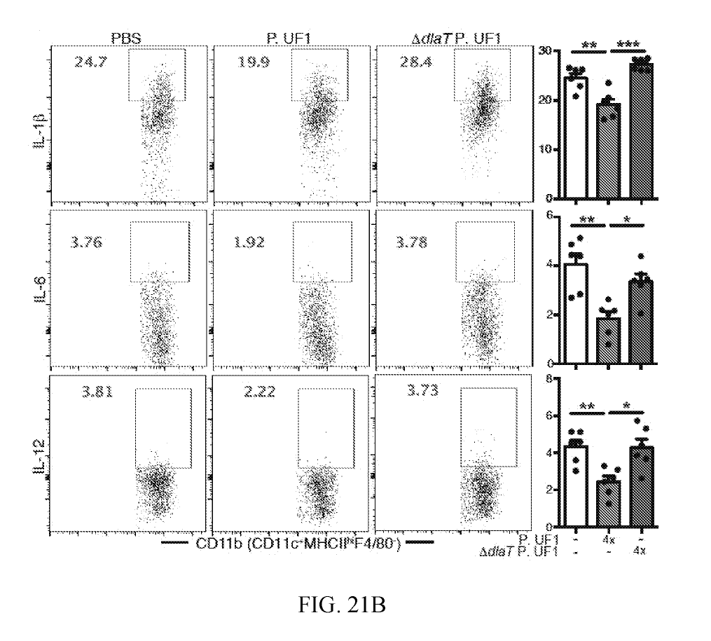

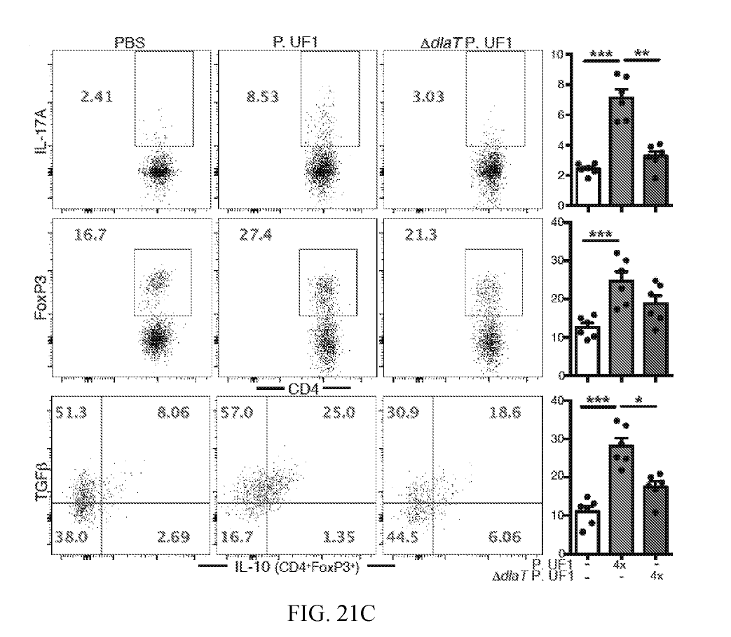

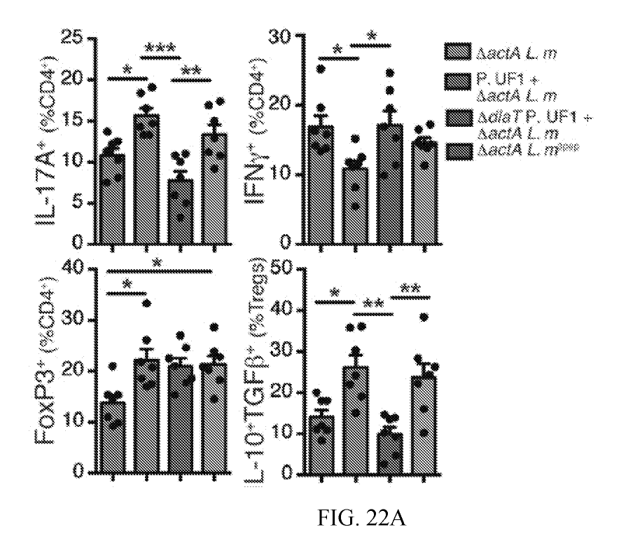

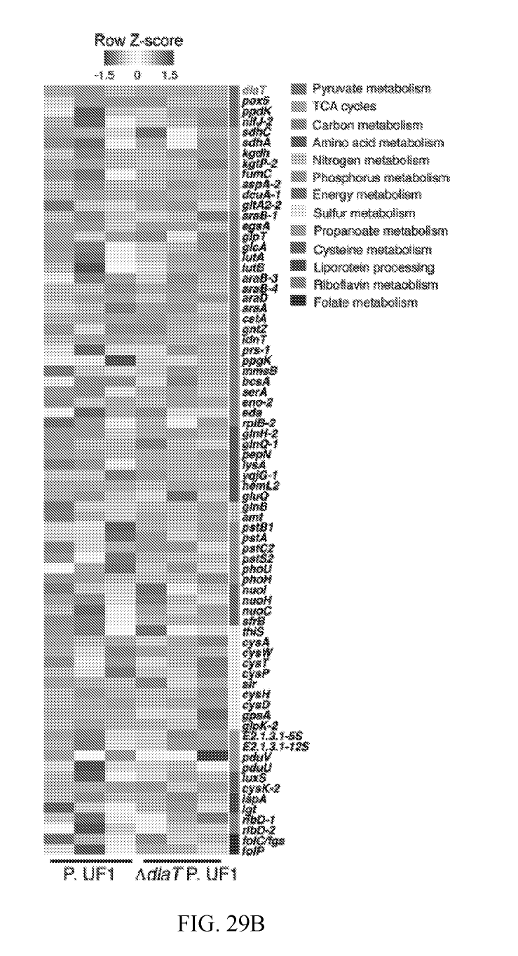

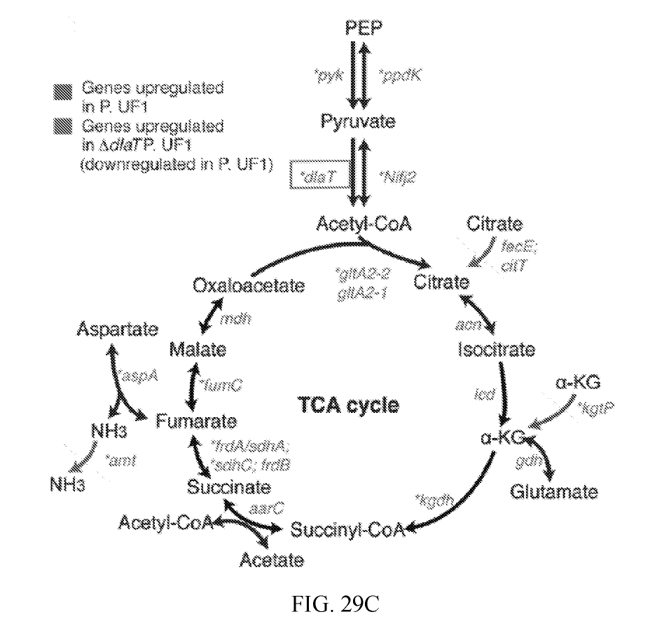

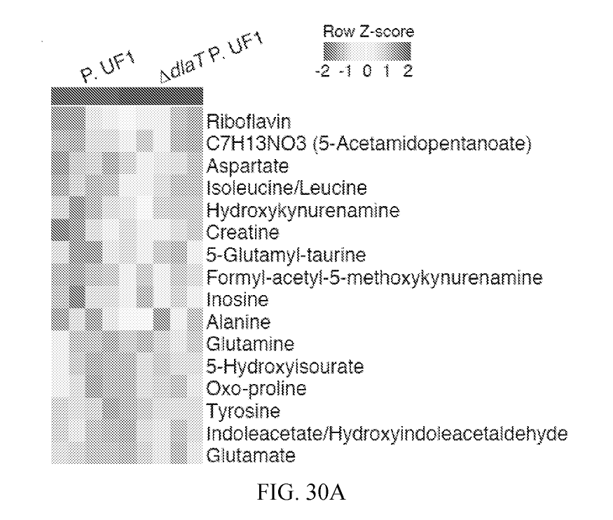

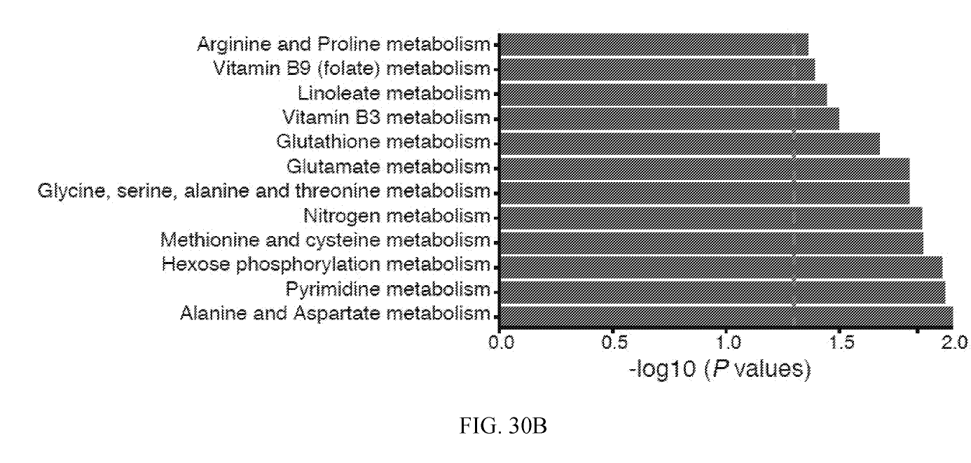

Induction of antigen-specific T cell responses, in general, and Th17 cell responses, in particular, are tightly controlled by microbial products along with critical cytokines (e.g., IL-6, TGFβ, IL-1β) that support the commitment and stabilization of T cell lineages against infection. To address the mechanistic paradigm associated with DlaT-specific Th17 cell responses, the dlaT gene was deleted from the bacterial chromosome by homologous recombination with a single crossover event resulting in the ΔdlaT P. UF1 strain (FIG. 21A). Subsequently, when compared to P. UF1 treatment of GF mice, GF mice monoassociated with ΔdlaT P. UF1 exhibited significantly increased levels of proinflammatory IL-1β, IL-6, and IL-12 in colonic DCs (FIG. 21B). The differentiation of Th17 cells was also abrogated, suggesting a crucial role for DlaT in the regulation of innate cells and Th17 cell formation (FIG. 21C). The mRNA transcriptomes and metabolites of P. UF1 and ΔdlaT P. UF1 strains were analyzed. Deletion of dlaT in P. UF1 resulted in significant changes in the expression of genes involved in the glycolytic and multiple metabolic pathways (e.g., the metabolism of carbohydrates, nitrogen, folate, and cysteine) (FIG. 29). Multiple tryptophan-derived metabolites (e.g., hydroxykynurenamine and formyl-acetyl-5-methoxykynurenamine) were significantly induced by P. UF1 when compared to ΔdlaT P. UF1 (FIG. 30), potentially influencing DC and T cell regulatory responses.

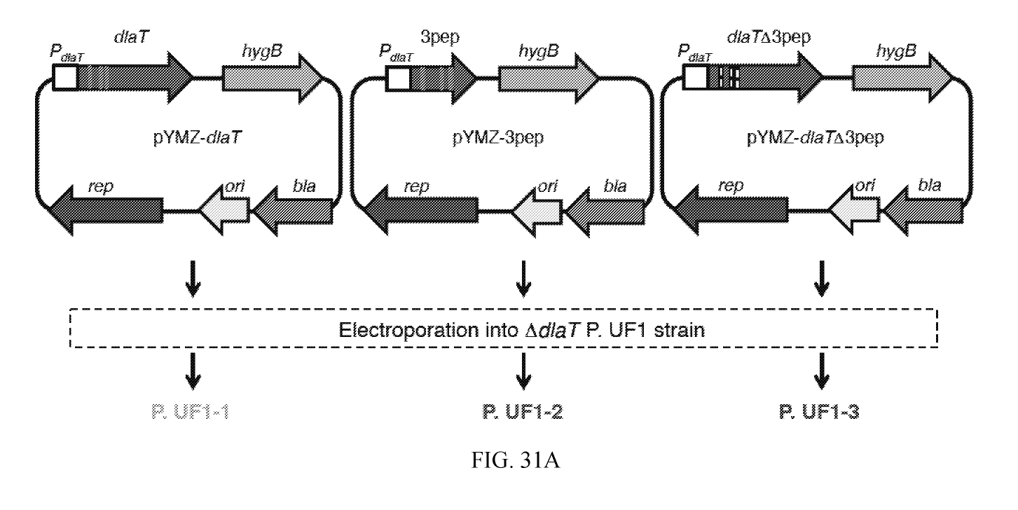

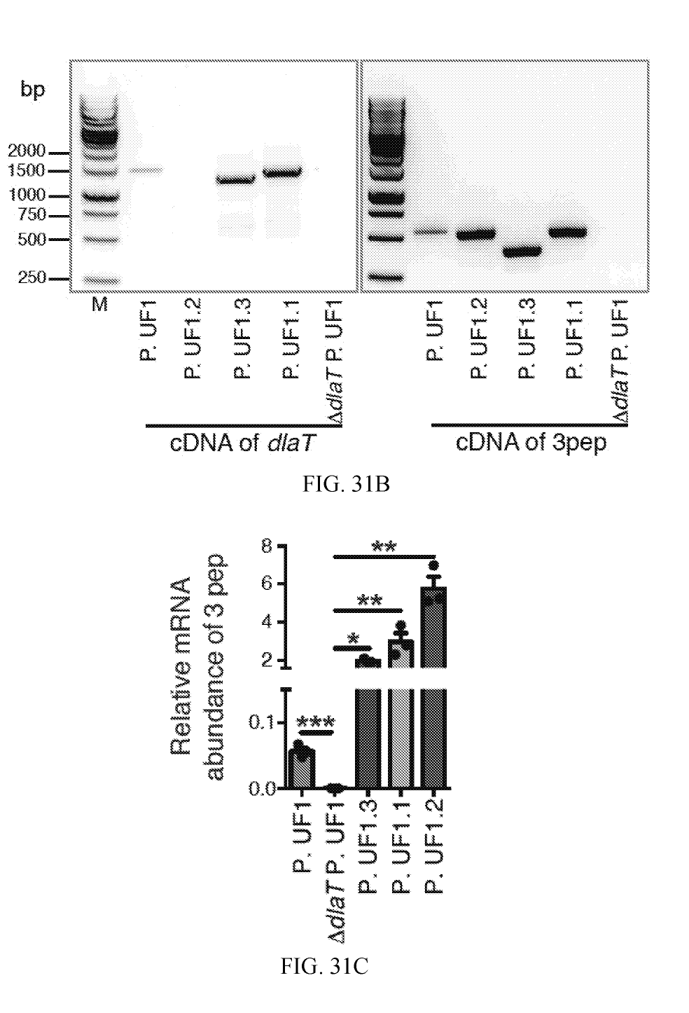

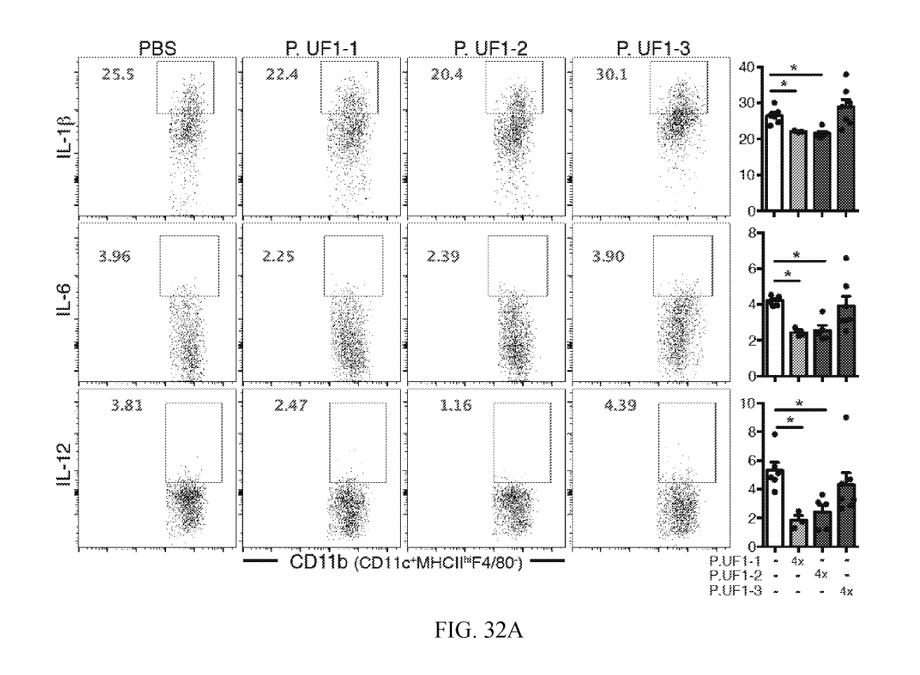

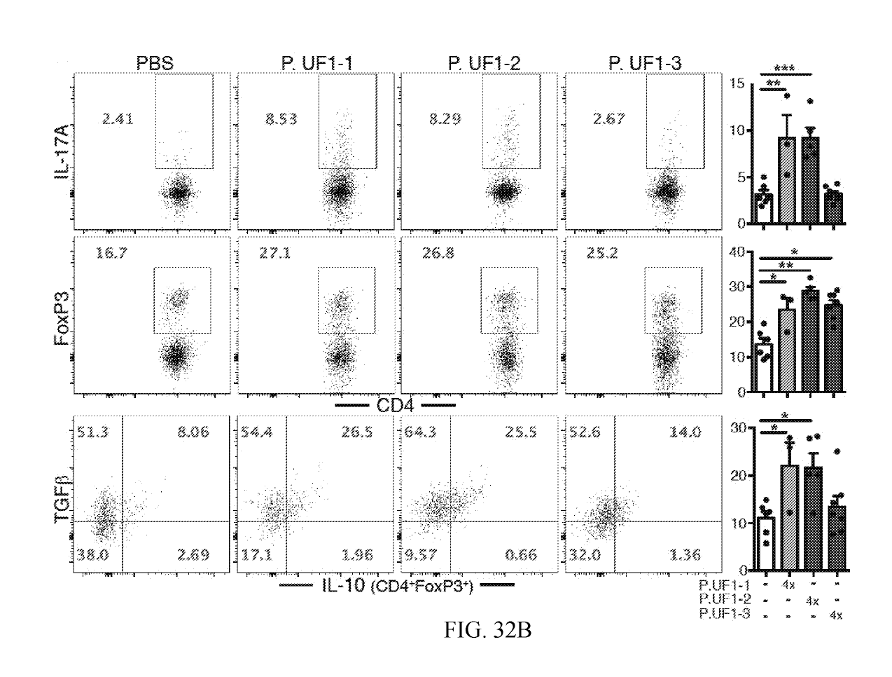

The dlaT gene, the three DlaT peptide sequences, or the dlaT gene without these three peptides were complemented in the ΔdlaT P. UF1 strain. The corresponding recombinant strains were designated as P. UF1-1, P. UF1-2, and P. UF1-3, respectively (FIGS. 31A-31C). GF mice were then monoassociated with P. UF1-1, P. UF1-2, or P. UF1-3. The mice receiving P. UF1-1 or P. UF1-2, but not P. UF1-3 with active dlaT minus the three complemented peptides, exhibited diminished proinflammatory responses by DCs and the induction of Th17 cell differentiation and functional IL-10⁺ Tregs (FIGS. 32A-32B) indicating that DlaT and the three peptides embedded within this bacterial protein are involved in the immune response.

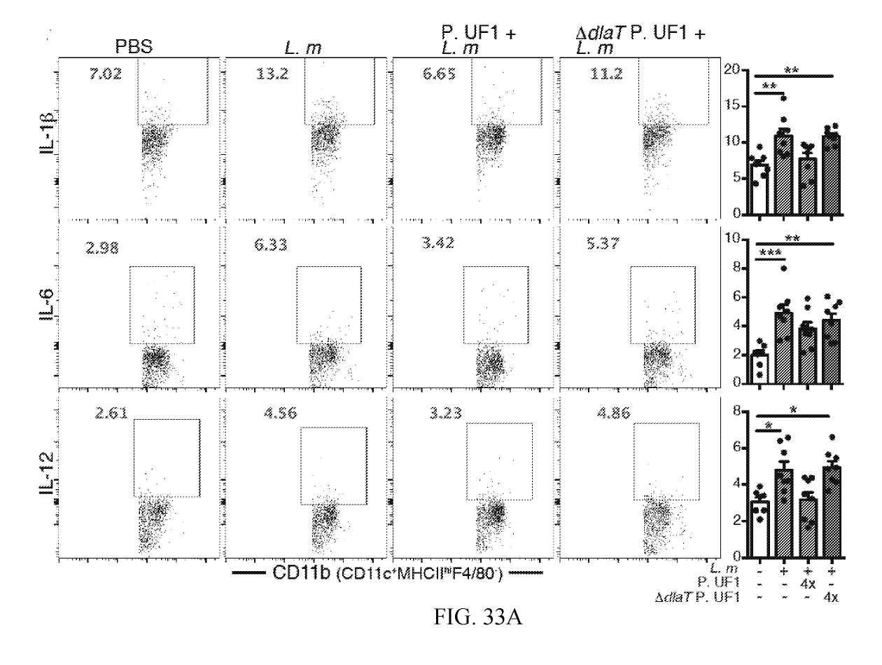

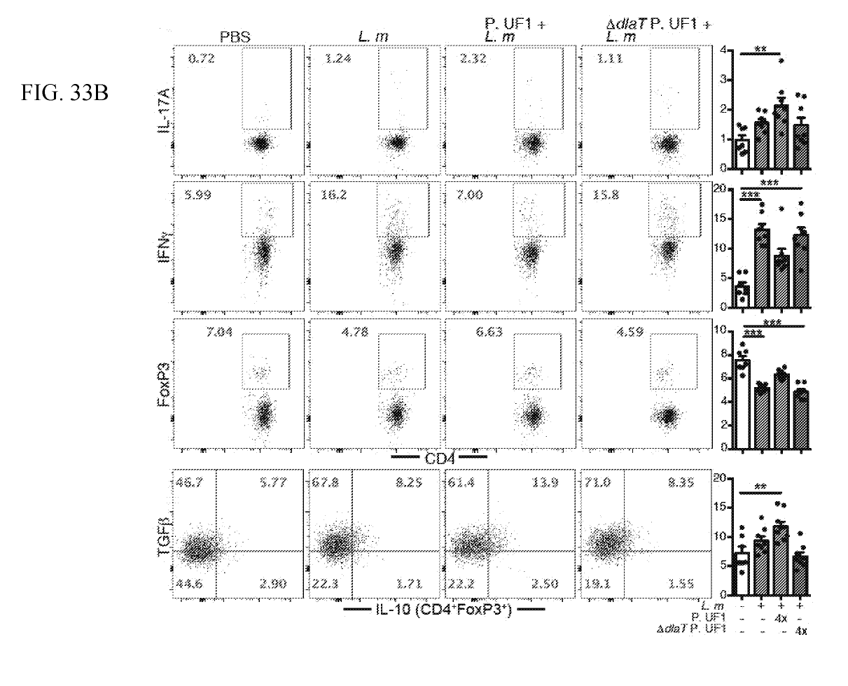

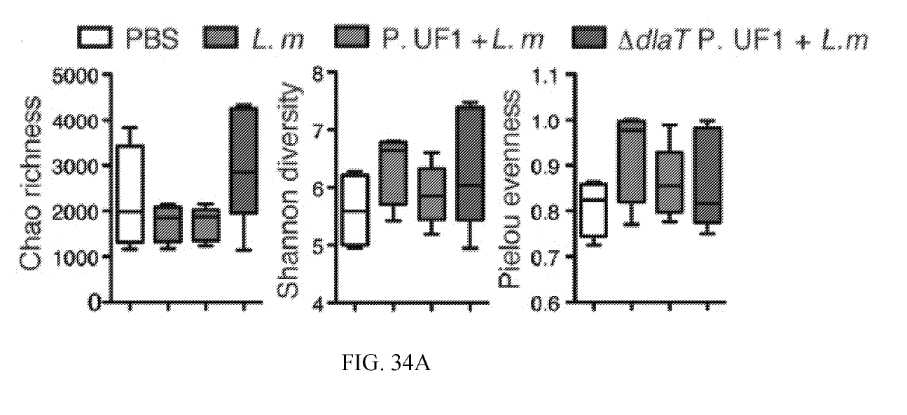

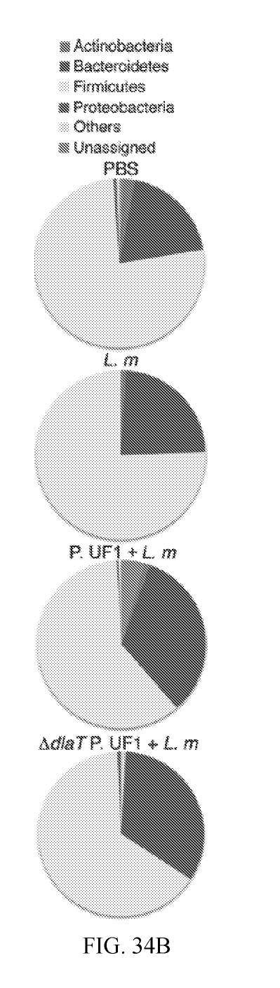

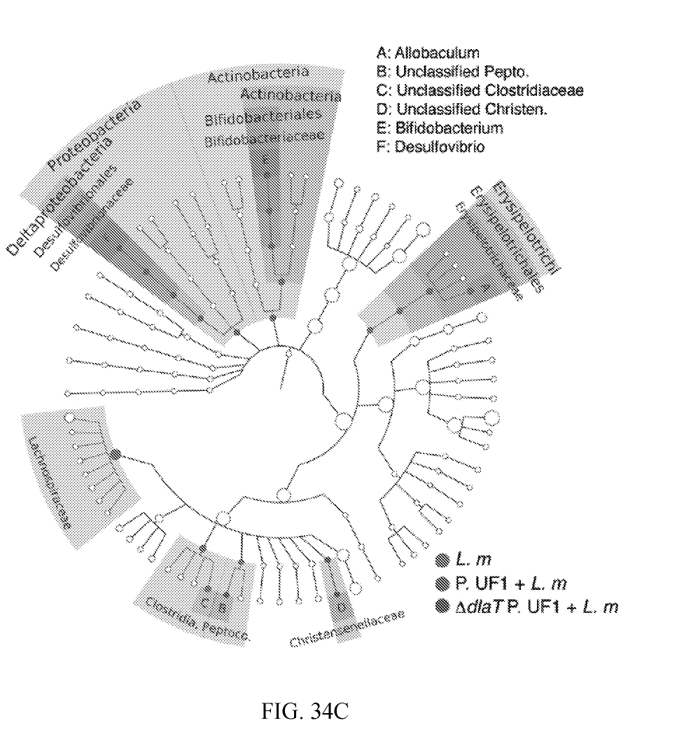

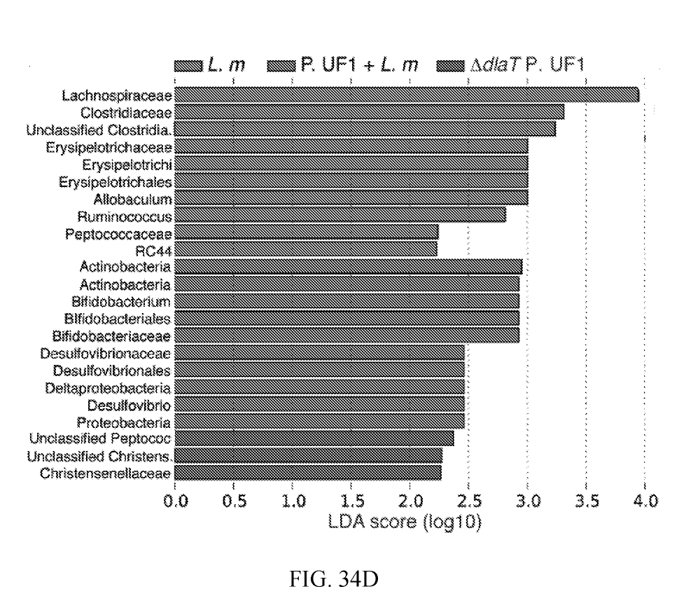

Listeria monocytogenes (L. m) expressing SFB peptides elicited Th1, but not Th17 cell responses. Lingering doubt about the role of IL-17 with L. m infection may reflect unrevealed facts about the Th17 cell responses to this pathogen. Mice were orally gavaged with P. UF1 and ΔdlaT P. UF1, and thereafter, infected with the murinized L. m strain 10403S containing targeted mutations in the major internalization protein, Internalin A (InlA)^{(S192N, Y363S)}, permitting optimal murine infection kinetics. Indeed, mice fed P. UF1 and then orally infected with L. m, when compared to mice gavaged with ΔdlaT P. UF1 or L. m-infected mice with no treatment, exhibited significantly reduced IL-1β, IL-6, and IL-12 by DCs (FIG. 33A), resulting in decreased IFNγ+Th1 cell responses, as well as increased induction of Th17 cells (FIG. 33B). Importantly, Tregs contract upon L. m infection; however, these cells were not only functionally sustained, but their numbers were also markedly increased in mice fed P. UF1 and infected with L. m, potentially contributing to protective T cell regulation (e.g., Th1 and Th17 cells) to attenuate inflammatory signals (FIG. 33B). The composition of the gut microbiota of mice fed P. UF1 and infected with L. m compared to the other groups demonstrated enrichment of bifidogenic bacteria (FIGS. 34A-34D), potentially positively influencing immune homeostasis during L. m infection.

### Example 7—the Role of Signr1 in Limiting Pathogen-Induced Inflammation

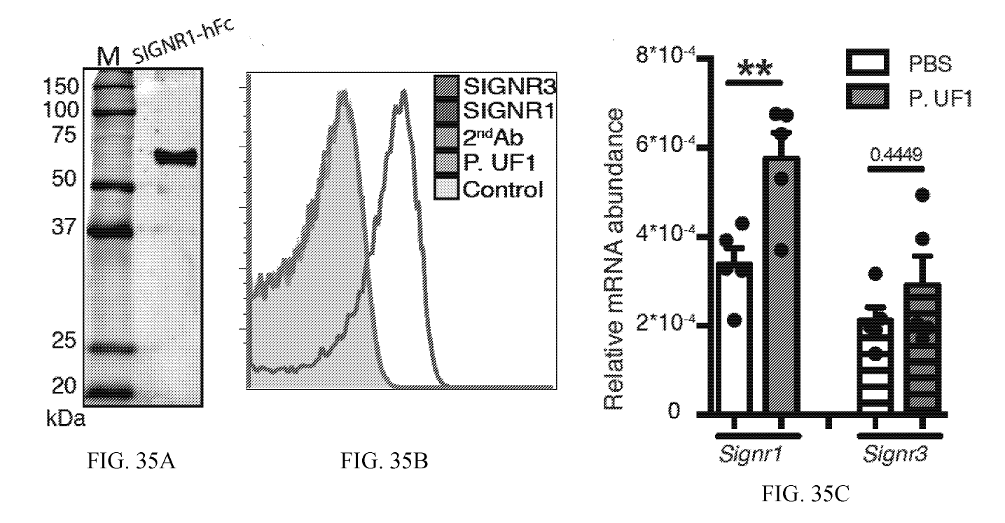

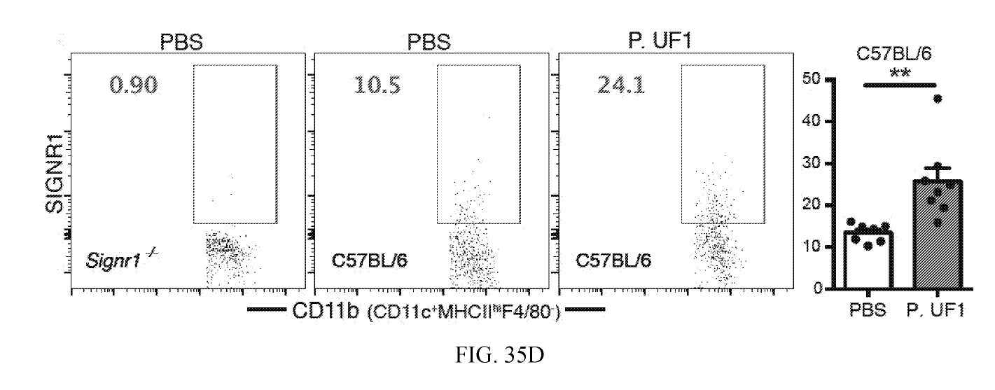

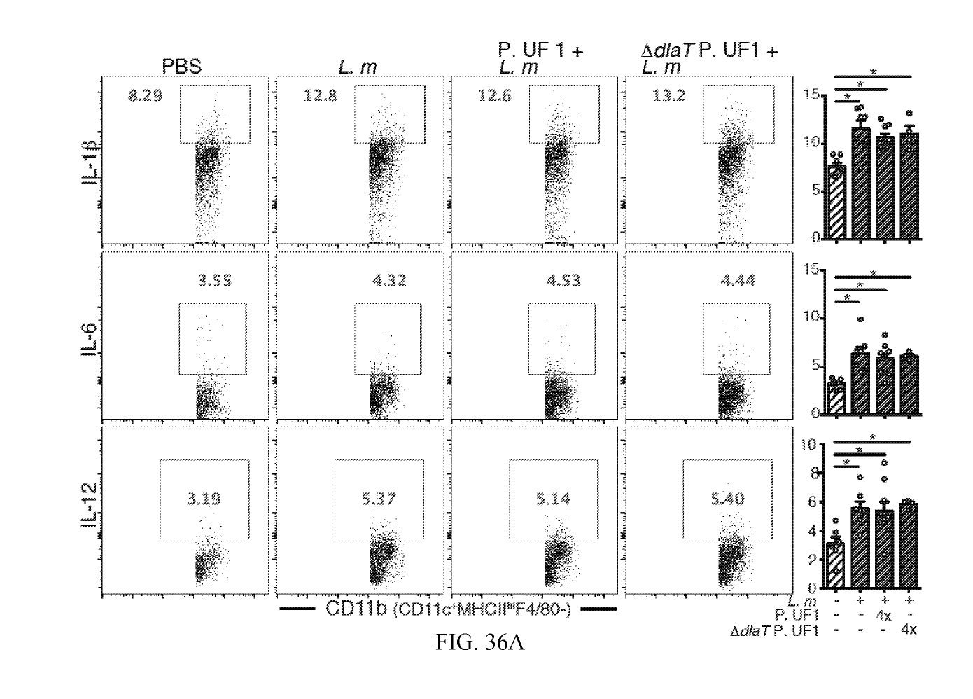

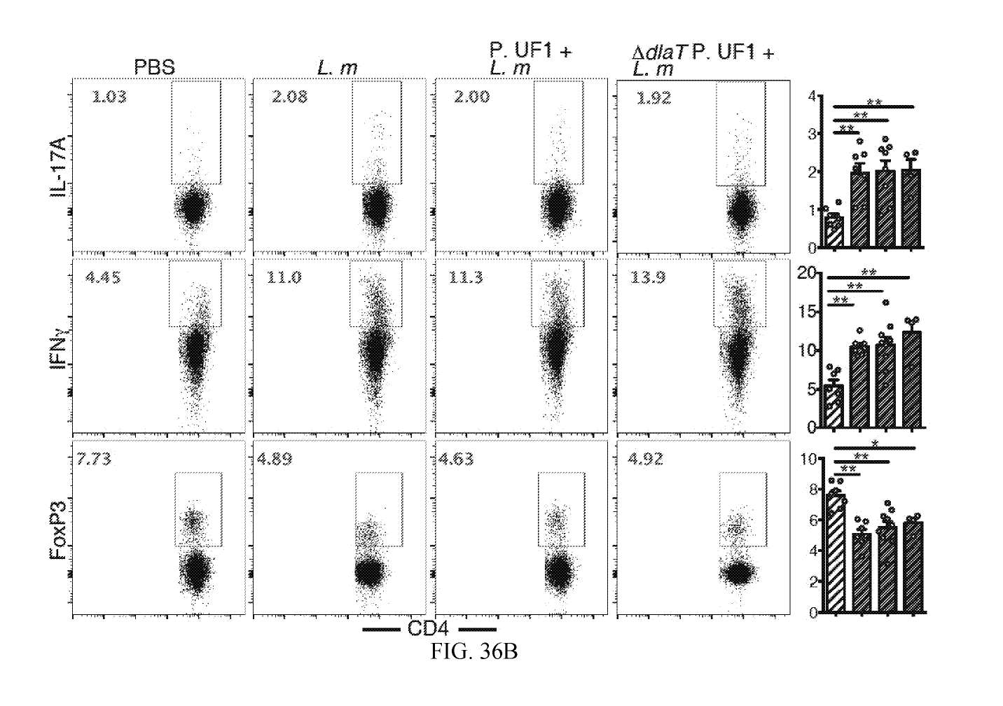

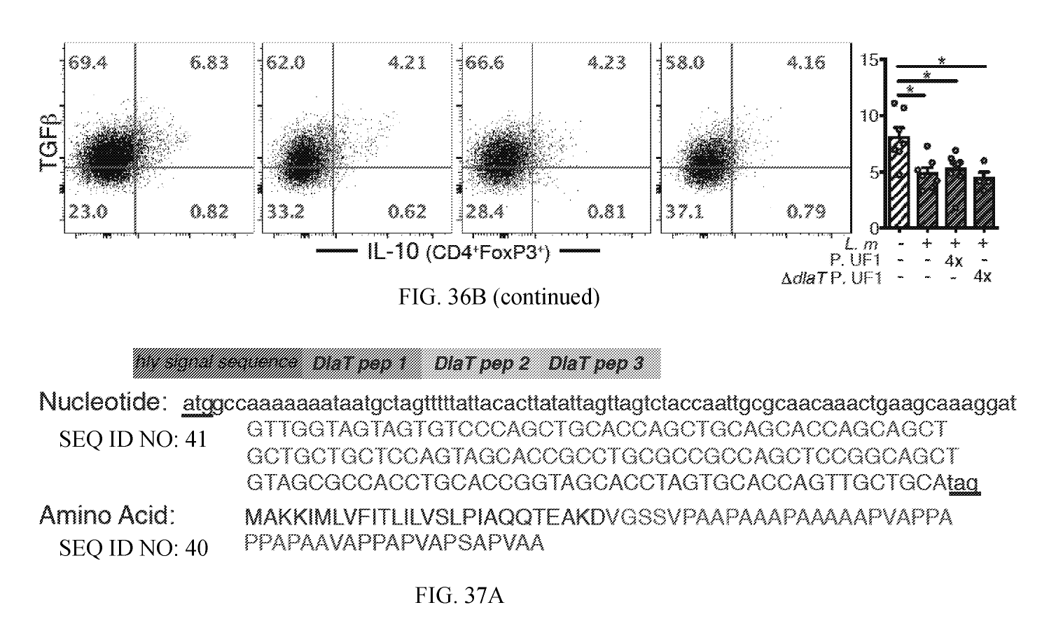

C-type lectin receptors (CLRs), particularly SIGNR1, recognize and internalize microbial products into their cellular compartments via glycan moieties to induce T cell differentiation. SIGNR1, but not SIGNR3, was identified as the binding receptor for P. UF1 when expressed by Chinese hamster ovary (CHO) cells in the form of a SIGNR1-hFc fusion protein (FIGS. 35A-35B). Additionally, SIGNR1 was constitutively expressed by colonic DCs, and was upregulated in mice orally treated with P. UF1 (FIGS. 35C-35D). To assess SIGNR1 involvement in limiting pathogen-induced inflammation, Signr1^{-/-} mice were gavaged with P. UF1, ΔdlaT P. UF1, or PBS, and then orally infected with L. m. SIGNR1 in assuages L. m-induced inflammation seen in wild type (wt) mice treated with P. UF1 (FIG. 33), which was not evident in P. UF1-treated Signr1^{-/-} mice (FIGS. 36A-36B). Thus, SIGNR1 has a regulatory role in pathogen infection.

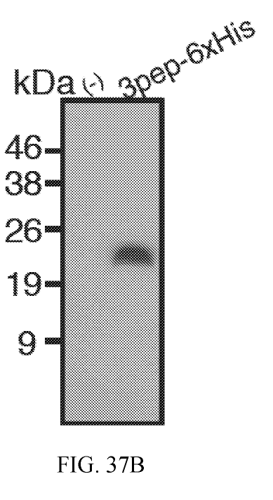

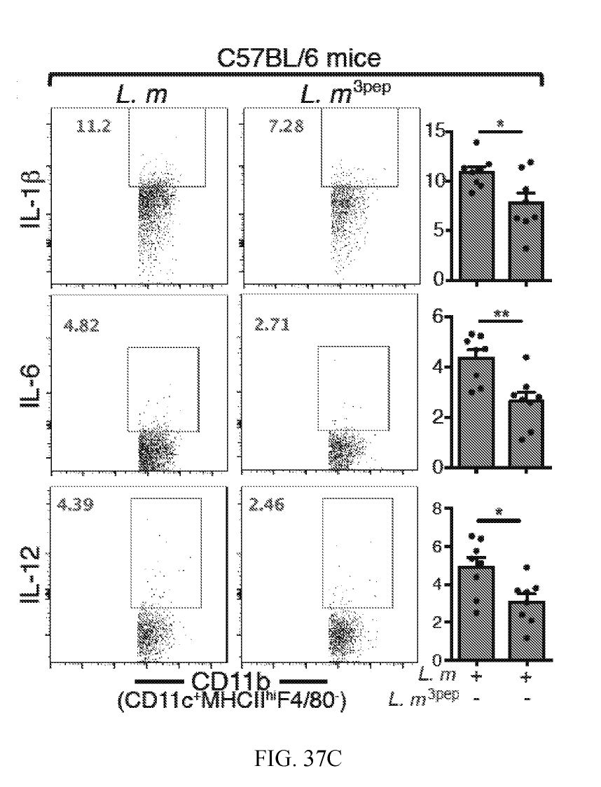

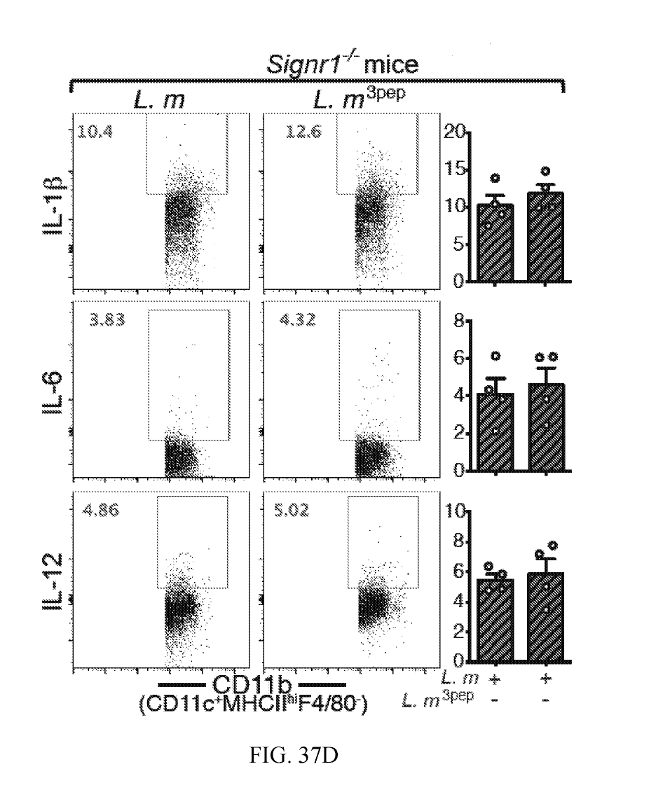

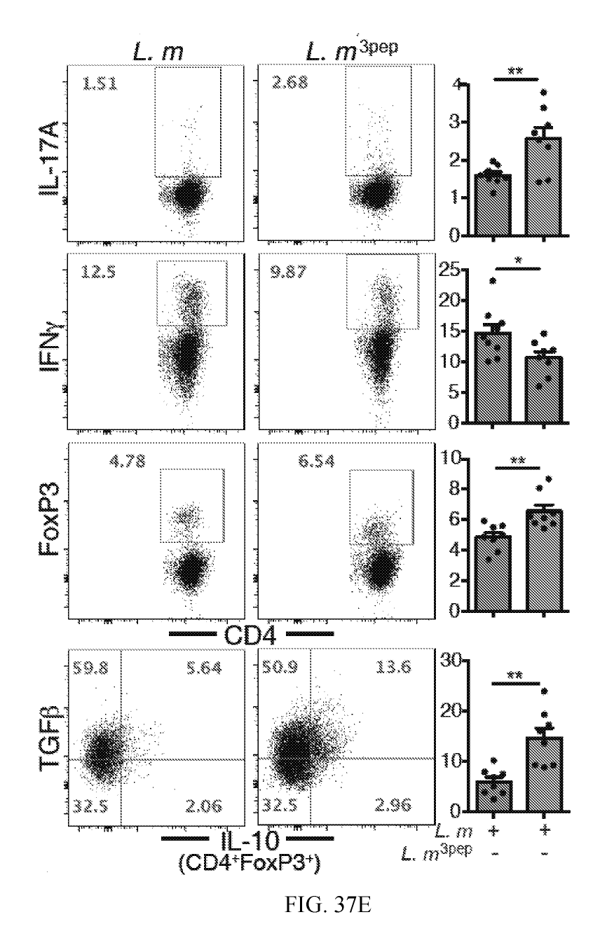

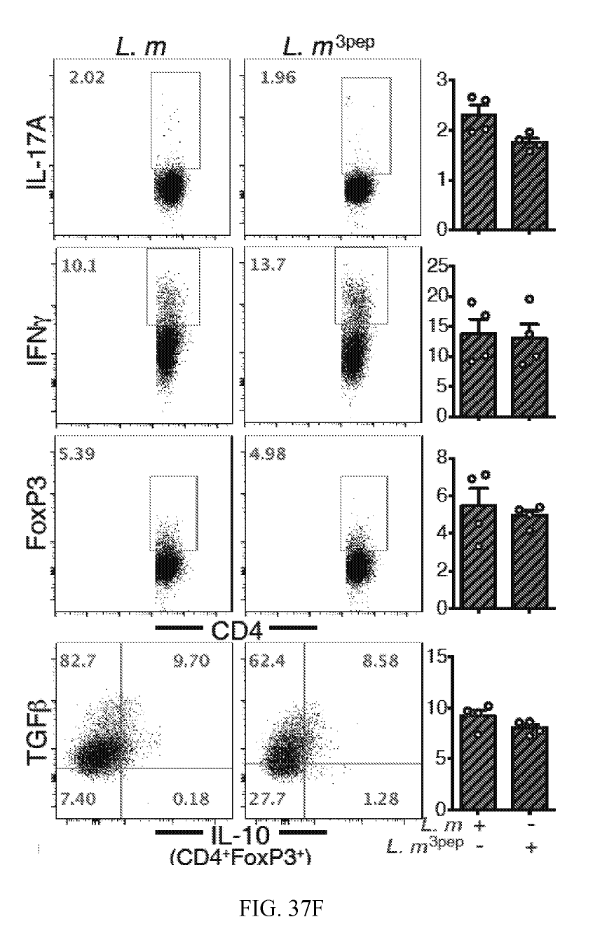

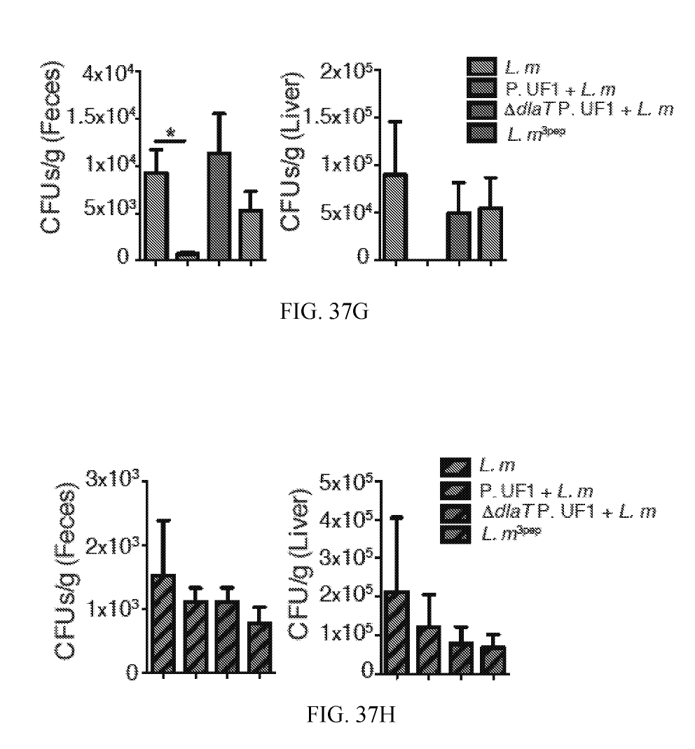

The three DlaT specific peptides were stably integrated into the L. m chromosome using the insertional expression vector, pIMK2, resulting in L. m^{3pep} (FIGS. 37A-37B). When compared to L. m infected mice, oral infection of wt mice with L. m^{3pep} resulted in dampened L. m-induced inflammation via reduction of proinflammatory cytokine levels (e.g., IL-1β), and decreased Th1 cell responses and generation of Th17 cells and Tregs (FIGS. 37C-37E). In contrast, this controlled immune protection was not observed in Signr1^{-/-} mice infected with L. m^{3pep} (FIGS. 37D-37F). Furthermore, L. m^{3pep} was rapidly disseminated, as shown by CFU counts in the fecal samples and tissues (e.g., liver) of wt mice when compared with L. m infected mice; however L. m^{3pep} burden was not abated in Signr1^{-/-} mice (FIGS. 37G-37H).

### Example 8—Impact of Intestinal L. M Infection on the Differentiation of Colonic Th17 Cells

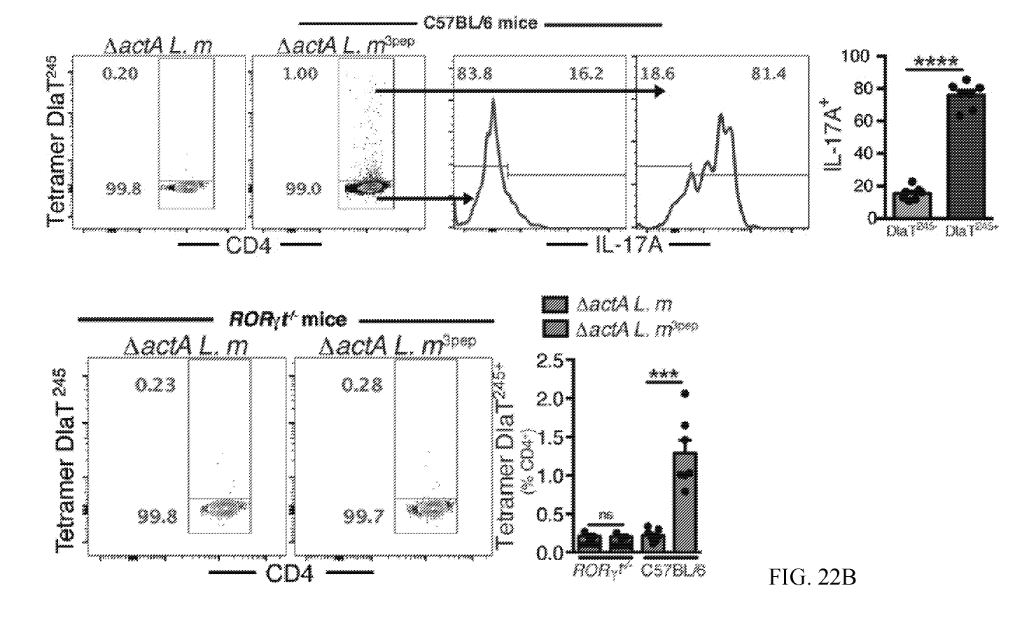

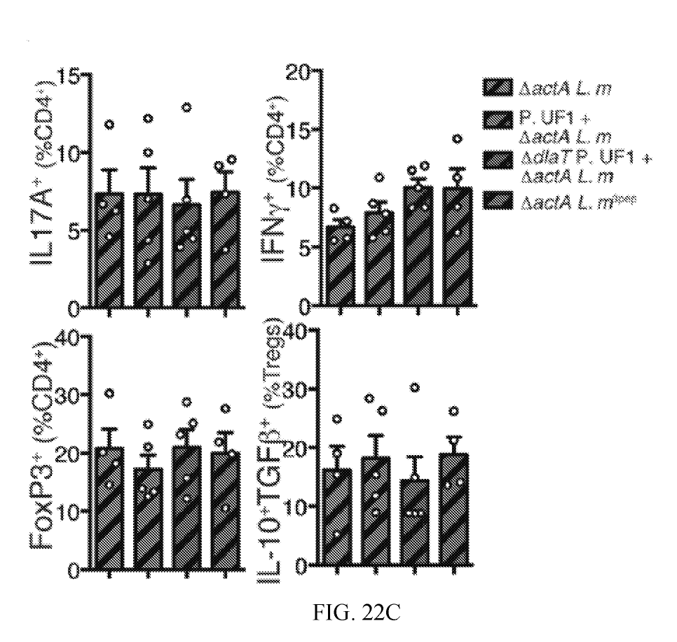

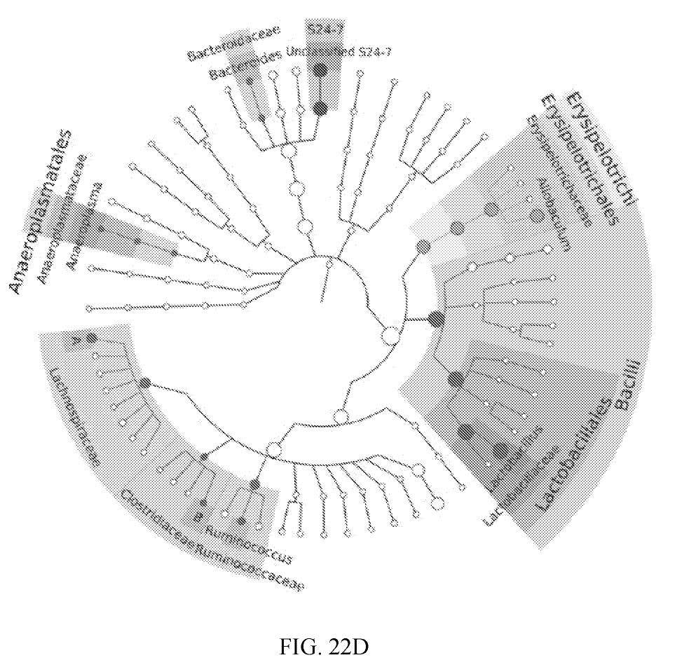

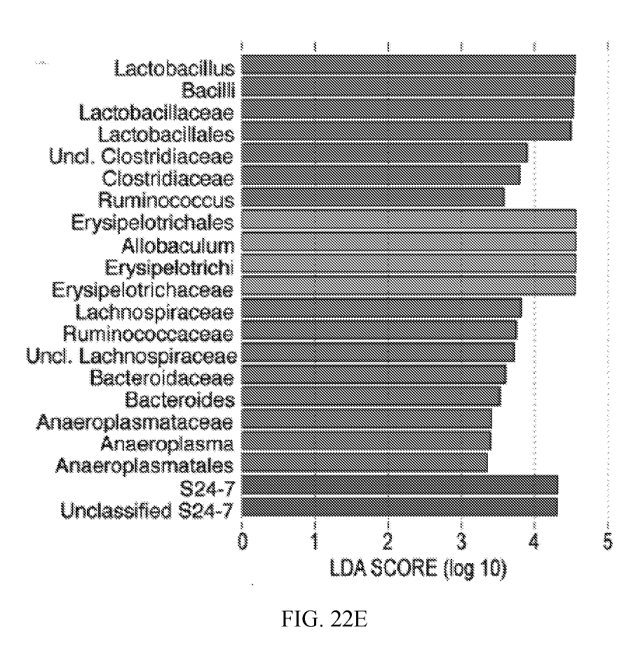

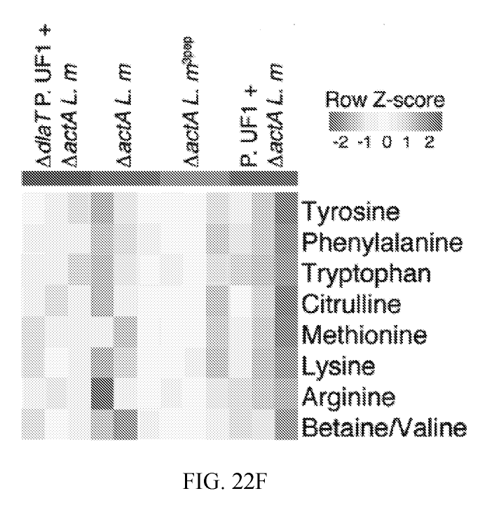

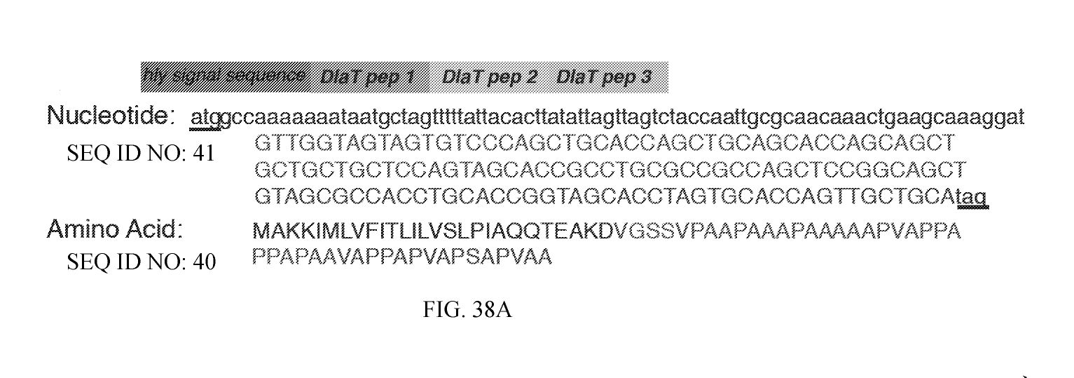

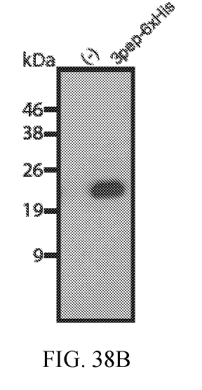

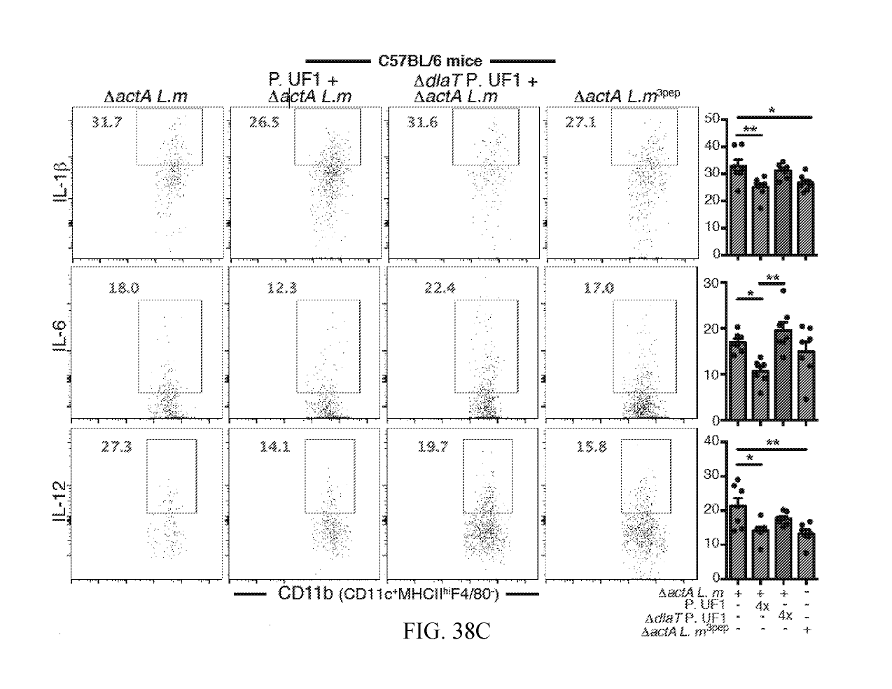

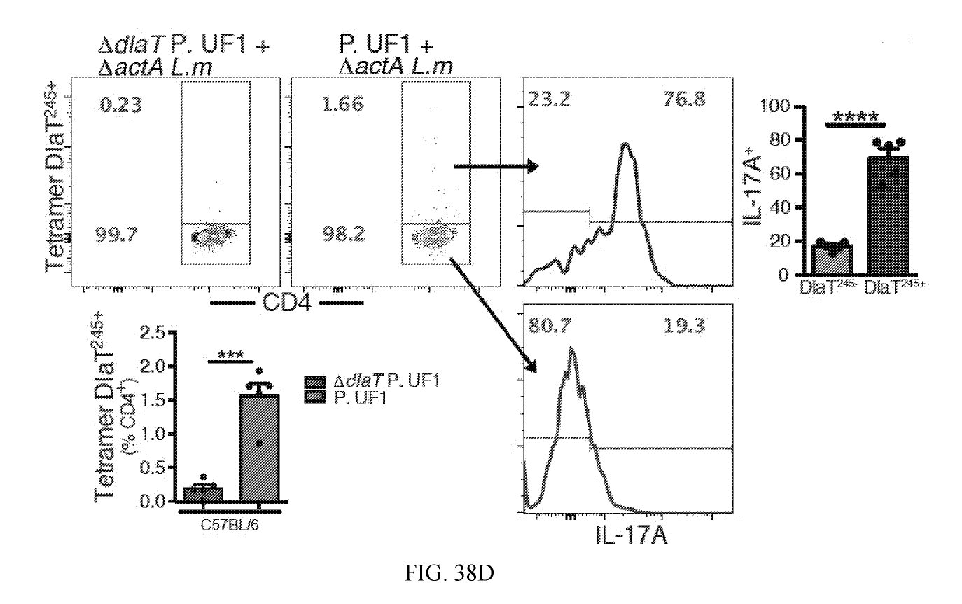

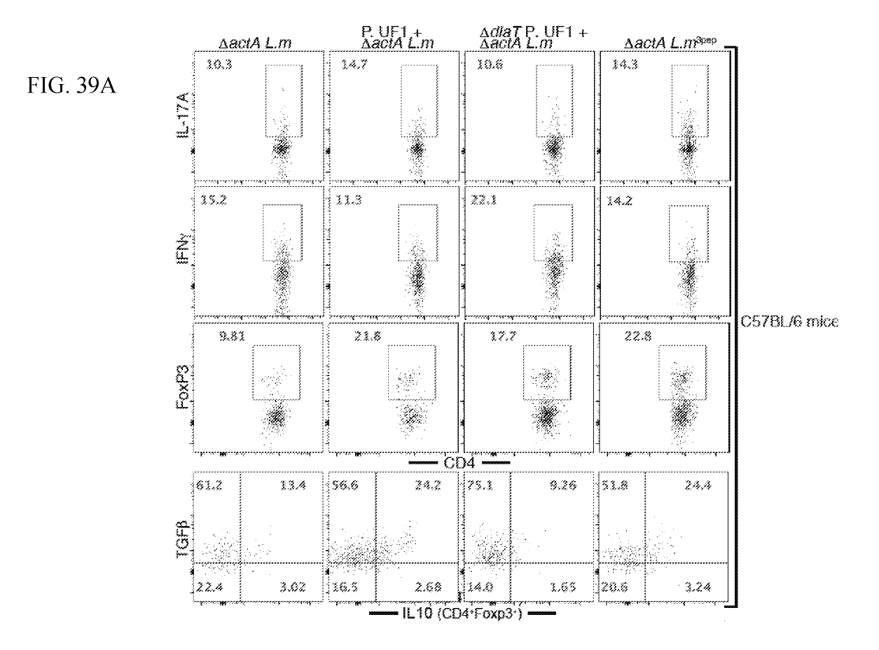

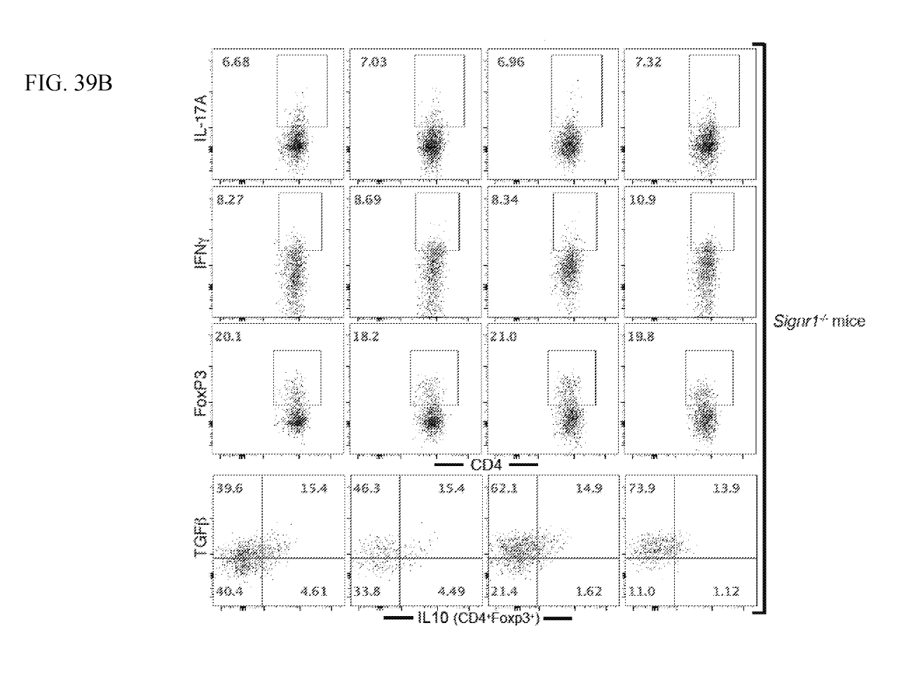

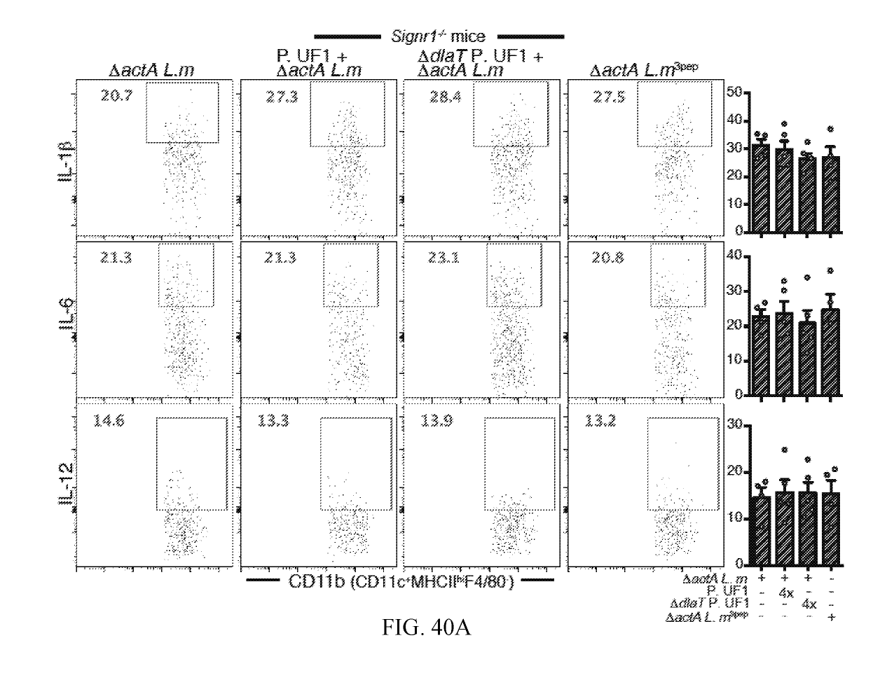

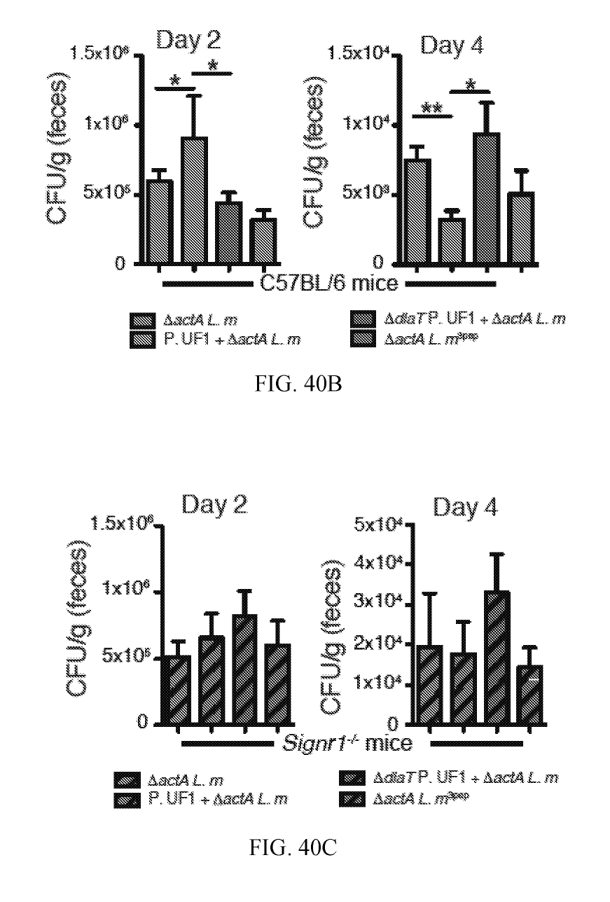

ΔactA L. m^{3pep} was generated by introducing pIMK2 harboring the three DlaT specific peptides into murinized 10403S ΔactA L. m (FIGS. 38A-38B). ΔactA L. m^{3pep}, like P. UF1, exerted control over DC activation (FIG. 34C), the induction of DlaT tetramer²⁴⁵⁺ Th17 cell formation, and the maintenance of functional IL-10⁺ Tregs when compared to other groups infected with ΔactA L. m or orally treated with ΔdlaT P. UF1 and infected with ΔactA L. m (FIGS. 22A-2B and 38D, 39A). These DlaT tetramer²⁴⁵⁺ Th17 cells were diminished in RORγt^{-/-} mice infected with ΔactA L. m^{3pep} (FIG. 22B). This highlights the involvement of DlaT in colonic Th17 cell differentiation, whereby its deletion resulted in a significant decrease of these cells upon ΔactA L. m infection (FIGS. 22A and 38D). Importantly, this trend was not observed in Signr1^{-/-} mice that were infected with ΔactA L. m^{3pep} or gavaged with P. UF1 or ΔdlaT P. UF1, and subsequently infected with ΔactA L. m (FIGS. 22C, 39B and 40A). Moreover, ΔactA L. m infection was significantly reduced in wt mice fed P. UF1 on day 4 when compared to other mouse groups (FIG. 40B). In contrast, reduction of L. m burden was not observed in any group of ΔactA L. m-infected Signr1^{-/-} mice (FIG. 40C). The composition of the gut microbiota of mice gavaged with P. UF1 and infected with ΔactA L. m when compared to that of other mouse groups demonstrates the enrichment of Lactobacillus and Ruminococcus species (FIGS. 22D-22E). This suggests that regulation of pathogen-induced inflammation by P. UF1 may not only play a role in reshaping the microbiota upon intestinal infection, but may also critically affect the pattern of induced metabolites (e.g., amino acids), including citrulline involved in nitric oxide production and innate signaling, ultimately controlling protective immune responses to ΔactA L. m infection. Such regulatory responses were not observed in ΔactA L. m or ΔdlaT P. UF1-treated mice (FIG. 22F).

### REFERENCES

1. Daniels, M. C. & Adair, L. S. Breast-feeding influences cognitive development in Filipino children. The Journal of nutrition 135, 2589-2595 (2005).
2. German, J. B., Dillard, C. J. & Ward, R. E. Bioactive components in milk. Curr Opin Clin Nutr Metab Care 5, 653-658 (2002).
3. Zivkovic, A. M., German, J. B., Lebrilla, C. B. & Mills, D. A. Human milk glycobiome and its impact on the infant gastrointestinal microbiota. Proceedings of the National Academy of Sciences of the United States of America 108 Suppl 1, 4653-4658 (2011).
4. Cerf-Bensussan, N. & Gaboriau-Routhiau, V. The immune system and the gut microbiota: friends or foes? Nature reviews. Immunology 10, 735-744 (2010).
5. Hooper, L. V. & Macpherson, A. J. Immune adaptations that maintain homeostasis with the intestinal microbiota. Nature reviews. Immunology 10, 159-169 (2010).
6. Garrett, W. S., Gordon, J. I. & Glimcher, L. H. Homeostasis and inflammation in the intestine. Cell 140, 859-870 (2010).
7. Kau, A. L., Ahern, P. P., Griffin, N. W., Goodman, A. L. & Gordon, J. I. Human nutrition, the gut microbiome and the immune system. Nature 474, 327-336 (2011).
8. Yang, T., Owen, J. L., Lightfoot, Y. L., Kladde, M. P. & Mohamadzadeh, M. Microbiota impact on the epigenetic regulation of colorectal cancer. Trends in molecular medicine 19, 714-725 (2013).
9. Goto, Y., et al. Segmented filamentous bacteria antigens presented by intestinal dendritic cells drive mucosal Th17 cell differentiation. Immunity 40, 594-607 (2014).
10. Yang, Y., et al. Focused specificity of intestinal TH17 cells towards commensal bacterial antigens. Nature 510, 152-156 (2014).
11. Mohamadzadeh, M., et al. Regulation of induced colonic inflammation by Lactobacillus acidophilus deficient in lipoteichoic acid. Proceedings of the National Academy of Sciences of the United States of America 108 Suppl 1, 4623-4630 (2011).
12. Lightfoot, Y. L. & Mohamadzadeh, M. Tailoring gut immune responses with lipoteichoic acid-deficient Lactobacillus acidophilus. Front Immunol 4, 25 (2013).
13. Kaser, A., Zeissig, S. & Blumberg, R. S. Inflammatory bowel disease. Annu Rev Immunol 28, 573-621 (2010).
14. Rescigno, M. Before they were gut dendritic cells. Immunity 31, 454-456 (2009).
15. Rescigno, M. Gut commensal flora: tolerance and homeostasis. F1000 Biol Rep 1, 9 (2009).
16. Rescigno, M. & Di Sabatino, A. Dendritic cells in intestinal homeostasis and disease. The Journal of clinical investigation 119, 2441-2450 (2009).
17. Ley, R. E., Peterson, D. A. & Gordon, J. I. Ecological and evolutionary forces shaping microbial diversity in the human intestine. Cell 124, 837-848 (2006).
18. Ley, R. E., Turnbaugh, P. J., Klein, S. & Gordon, J. I. Microbial ecology: human gut microbes associated with obesity. Nature 444, 1022-1023 (2006).
19. Lee, Y. K. & Mazmanian, S. K. Has the microbiota played a critical role in the evolution of the adaptive immune system? Science 330, 1768-1773 (2010).
20. Ivanov, I I, et al. Induction of intestinal Th17 cells by segmented filamentous bacteria. Cell 139, 485-498 (2009).
21. Melmed, G. Y. & Targan, S. R. Future biologic targets for IBD: potentials and pitfalls. Nat Rev Gastroenterol Hepatol 7, 110-117 (2010).
22. Lasiglie, D., et al. Role of IL-1 beta in the development of human T(H)17 cells: lesson from NLPR3 mutated patients. PLoS One 6, e20014 (2011).
23. Baumgart, D. C. & Sandborn, W. J. Inflammatory bowel disease: clinical aspects and established and evolving therapies. Lancet 369, 1641-1657 (2007).
24. Baumgart, D. C. & Carding, S. R. Inflammatory bowel disease: cause and immunobiology. Lancet 369, 1627-1640 (2007).
25. Eun, C. S., et al. Induction of bacterial antigen-specific colitis by a simplified human microbiota consortium in gnotobiotic interleukin-10−/− mice. Infection and immunity 82, 2239-2246 (2014).
26. Goldsmith, J. R. & Sartor, R. B. The role of diet on intestinal microbiota metabolism: downstream impacts on host immune function and health, and therapeutic implications. Journal of gastroenterology 49, 785-798 (2014).
27. Sartor, R. B. The intestinal microbiota in inflammatory bowel diseases. Nestle Nutr Inst Workshop Ser 79, 29-39 (2014).
28. David, L. A., et al. Diet rapidly and reproducibly alters the human gut microbiome. Nature 505, 559-563 (2014).
29. Johnson, J. L. & Cummins, C. S. Cell wall composition and deoxyribonucleic acid similarities among the anaerobic coryneforms, classical propionibacteria, and strains of Arachnia propionica. Journal of bacteriology 109, 1047-1066 (1972).
30. Falentin, H., et al. The complete genome of Propionibacterium freudenreichii CIRM-BIA1, a hardy actinobacterium with food and probiotic applications. PLoS One 5, e11748 (2010).
31. Foligne, B., et al. Promising immunomodulatory effects of selected strains of dairy propionibacteria as evidenced in vitro and in vivo. Applied and environmental microbiology 76, 8259-8264 (2010).
32. Urbaniak, C., et al. Effect of chemotherapy on the microbiota and metabolome of human milk, a case report. Microbiome 2, 24 (2014).
33. Gringhuis, S. I., den Dunnen, J., Litjens, M., van der Vlist, M. & Geijtenbeek, T. B. Carbohydrate-specific signaling through the D C-SIGN signalosome tailors immunity to Mycobacterium tuberculosis, HIV-1 and Helicobacter pylori. Nature immunology 10, 1081-1088 (2009).
34. Geijtenbeek, T. B., van Vliet, S. J., Engering, A., t Hart, B. A. & van Kooyk, Y. Self- and nonself-recognition by C-type lectins on dendritic cells. Annual review of immunology 22, 33-54 (2004).
35. Robinson, M. J., Sancho, D., Slack, E. C., LeibundGut-Landmann, S. & Reis e Sousa, C. Myeloid C-type lectins in innate immunity. Nature immunology 7, 1258-1265 (2006).
36. Varol, C., et al. Intestinal lamina propria dendritic cell subsets have different origin and functions. Immunity 31, 502-512 (2009).
37. Prabagar, M. G., et al. SIGN-R1, a C-type lectin, enhances apoptotic cell clearance through the complement deposition pathway by interacting with C1q in the spleen. Cell death and differentiation 20, 535-545 (2013).
38. Kang, Y. S., et al. A dominant complement fixation pathway for pneumococcal polysaccharides initiated by SIGN-R1 interacting with C1q. Cell 125, 47-58 (2006).
39. Belkaid, Y. Paradoxical roles of Foxp3+ T cells during infection: from regulators to regulators. Cell Host Microbe 3, 341-343 (2008).
40. Belkaid, Y. & Oldenhove, G. Tuning microenvironments: induction of regulatory T cells by dendritic cells. Immunity 29, 362-371 (2008).
41. Devkota, S., et al. Dietary-fat-induced taurocholic acid promotes pathobiont expansion and colitis in 110−/− mice. Nature 487, 104-108 (2012).
42. Furusawa, Y., et al. Commensal microbe-derived butyrate induces the differentiation of colonic regulatory T cells. Nature 504, 446-450 (2013).
43. Ivanov, I I & Honda, K. Intestinal commensal microbes as immune modulators. Cell host & microbe 12, 496-508 (2012).
44. Mohamadzadeh, M., et al. Freshly isolated mouse 4F7+ splenic dendritic cells process and present exogenous antigens to T cells. European journal of immunology 24, 3170-3174 (1994).
45. Wang, Z., et al. Regulatory T cells promote a protective Th17-associated immune response to intestinal bacterial infection with C. rodentium. Mucosal immunology 7, 1290-1301 (2014).
46. Coccia, M., et al. IL-1beta mediates chronic intestinal inflammation by promoting the accumulation of IL-17A secreting innate lymphoid cells and CD4(+) Th17 cells. The Journal of experimental medicine 209, 1595-1609 (2012).
47. Murai, M., et al. Interleukin 10 acts on regulatory T cells to maintain expression of the transcription factor Foxp3 and suppressive function in mice with colitis. Nat Immunol 10, 1178-1184 (2009).

1-107. (canceled)

108. A composition comprising an infant food and a pharmaceutically effective amount of a probiotic bacterium, said pharmaceutically effective amount of probiotic bacterium comprising about 10⁴ to about 10¹³ CFU/Kg of infant body weight.

109. The composition according to claim 108, wherein the probiotic bacterium is P. freudenreichii.

110. The composition according to claim 108, wherein the infant food is donor milk, maternal milk, solid infant food or formula.

111. A method of promoting development of intestinal microbiota and/or inducing protective and regulatory immunity comprising administering a composition according to claim 108 to a human or non-human mammalian offspring.

112. A method of promoting development of intestinal microbiota and/or inducing protective and regulatory immunity in human or non-human mammalian offspring comprising administering a composition according to claim 108 to a nursing or lactating female and supplying milk from said nursing or lactating female to human infant or non-human mammalian offspring.

113. A composition comprising an infant food and a pharmaceutically effective amount of one or more peptides selected from SEQ ID NO: 1, SEQ ID NO: 2, SEQ ID NO: 3, SEQ ID NOs: 42-48, fragments of SEQ ID NO: 48 or a combination thereof, wherein the pharmaceutically effective amount of the one or more peptides induce protective and/or regulatory immunity in a human or non-human mammalian offspring.

114. A method of inducing protective and/or regulatory immunity in a human or non-human mammalian offspring, the method comprising administering a composition according to claim 113 the human or non-human mammalian offspring.

115. A composition comprising one or more peptides selected from SEQ ID NO: 1, SEQ ID NO: 2, SEQ ID NO: 3, SEQ ID NOs: NOs: 42-48, and fragments of SEQ ID NO: 48 and buffered aqueous solution, capsule, pill, powder, granule, a solution or a suspension in an aqueous or non-aqueous liquid, an elixir or a syrup.

116. A method of promoting development of intestinal microbiota and/or inducing protective and regulatory immunity comprising administering a composition according to claim 115 to a human or non-human.

117. A method of promoting development of intestinal microbiota and/or inducing protective and regulatory immunity comprising administering a composition comprising a pharmaceutically effective amount of a probiotic bacterium to a human or non-human.

118. A recombinant bacterial cell comprising a vector, said vector comprising DNA operably linked to a promoter, said DNA encoding SEQ ID NO: 1, SEQ ID NO: 2, SEQ ID NO: 3, SEQ ID NOs: 42-47, SEQ ID NO: 48 or fragments of SEQ ID NO: 48.

119. The recombinant bacterial cell according to claim 118, wherein said bacterial cell overexpresses a protein or peptide selected from SEQ ID NO: 1, SEQ ID NO: 2, SEQ ID NO: 3, SEQ ID NOs: 42-47, SEQ ID NO: 48 or fragments of SEQ ID NO: 48.

120. A composition comprising the recombinant bacterial cell according to claim 118.

121. The composition according to claim 120, wherein said composition is a food product.

122. A method of promoting development of intestinal microbiota and/or inducing protective and regulatory immunity comprising administering a composition comprising a recombinant bacterial cell according to claim 118 to a human or non-human.

123. The method according to claim 122, wherein said microbial cell overexpresses a protein or peptide selected from SEQ ID NO: 1, SEQ ID NO: 2, SEQ ID NO: 3, SEQ ID NOs: 42-47, SEQ ID NO: 48 or fragments of SEQ ID NO: 48.

124. The method according to claim 122, wherein said composition is in the form of a food.


SEQUENCE LISTING (48 sequences)

SEQ ID NO: 1 - protein, 15 aa, Artificial Sequence
  DlaT 208-222
VGSSVPAAPAAAPAA

SEQ ID NO: 2 - protein, 15 aa, Artificial Sequence
  DlaT 245-259
VAPPAPVAPSAPVAA

SEQ ID NO: 3 - protein, 15 aa, Artificial Sequence
  DlaT 109-123
AAAPVAPPAPPAPAA

SEQ ID NO: 4 - protein, 15 aa, Artificial Sequence
  control tetramer
PVSKMRMATPLLMQA

SEQ ID NO: 5 - DNA, 29 bp, Artificial Sequence
  DlaT-F
cccaagcttgaggtggctgaaggaagttg

SEQ ID NO: 6 - DNA, 29 bp, Artificial Sequence
  DlaT-R
cccggatcctcctccttgacgtggatctc

SEQ ID NO: 7 - DNA, 32 bp, Artificial Sequence
  CdlaT-F
ccctctagaccagcttctcgtgacactcattc

SEQ ID NO: 8 - DNA, 26 bp, Artificial Sequence
  CdlaT-R
cccggatcccgcgatccccgttacgg

SEQ ID NO: 9 - DNA, 39 bp, Artificial Sequence
  3Pep-lap-R
cggctgcgggaatcggcatggttctacgtgactcctttg

SEQ ID NO: 10 - DNA, 39 bp, Artificial Sequence
  3Pep-lap-F
caaaggagtcacgtagaaccatgccgattcccgcagccg

SEQ ID NO: 11 - DNA, 29 bp, Artificial Sequence
  3Pep-R
cccggatccctaagccggtgggttcaccg

SEQ ID NO: 12 - DNA, 32 bp, Artificial Sequence
  Pep1-lap-F
ccccgattcccgcagccgcggcacctgccggc

SEQ ID NO: 13 - DNA, 32 bp, Artificial Sequence
  Pep1-lap-R
gccggcaggtgccgcggctgcgggaatcgggg

SEQ ID NO: 14 - DNA, 32 bp, Artificial Sequence
  Pep2-lap-F
ccgtactcggcgtcgtccctgcggcacccgcc

SEQ ID NO: 15 - DNA, 32 bp, Artificial Sequence
  Pep2-lap-R
ggcgggtgccgcagggacgacgccgagtacgg

SEQ ID NO: 16 - DNA, 30 bp, Artificial Sequence
  Pep3-lap-F
gcggctccggcccccccgcccgcggcgccg

SEQ ID NO: 17 - DNA, 30 bp, Artificial Sequence
  Pep3-lap-R
cggcgccgcgggcgggggggccggagccgc

SEQ ID NO: 18 - DNA, 39 bp, Artificial Sequence
  3Pep-F2
caacaaactgaagcaaaggatgttggtagtagtgtccca

SEQ ID NO: 19 - DNA, 29 bp, Artificial Sequence
  3Pep-R2
atatgtcgacctatgcagcaactggtgca

SEQ ID NO: 20 - DNA, 37 bp, Artificial Sequence
  HlySS-F
atatccatggccaaaaaaataatgctagtttttatta

SEQ ID NO: 21 - DNA, 39 bp, Artificial Sequence
  HlySS-R
tgggacactactaccaacatcctttgcttcagtttgttg

SEQ ID NO: 22 - DNA, 28 bp, Artificial Sequence
  Signr1exp-F
ccggaattcgcaagtctccaaaacccca

SEQ ID NO: 23 - DNA, 32 bp, Artificial Sequence
  Signr1exp-R
catgccatggggccttcagtgcatggggttgc

SEQ ID NO: 24 - DNA, 20 bp, Artificial Sequence
  C. rodentium-F
aagtctgtcaataccgcctc

SEQ ID NO: 25 - DNA, 20 bp, Artificial Sequence
  C. rodentium-R
aatgtgccaactgtctcatc

SEQ ID NO: 26 - DNA, 21 bp, Artificial Sequence
  P1
gtcttctgccccatacgctaa

SEQ ID NO: 27 - DNA, 21 bp, Artificial Sequence
  P2
gatgtcctcggagtcgtggta

SEQ ID NO: 28 - DNA, 19 bp, Artificial Sequence
  P3
caccctgacggagatccac

SEQ ID NO: 29 - DNA, 17 bp, Artificial Sequence
  P4
ccgacgacgccgagtac

SEQ ID NO: 30 - DNA, 19 bp, Artificial Sequence
  GroL2-RT-F
caatgtcgtgttggagaag

SEQ ID NO: 31 - DNA, 18 bp, Artificial Sequence
  GroL2-RT-R
cgccgatcttgtggtagg

SEQ ID NO: 32 - DNA, 20 bp, Artificial Sequence
  Gapdh-RT-F
ggtgaaggtcggtgtgaacg

SEQ ID NO: 33 - DNA, 20 bp, Artificial Sequence
  Gapdh-RT-R
ctcgctcctggaagatggtg

SEQ ID NO: 34 - DNA, 21 bp, Artificial Sequence
  Signr1-RT-F
ttgatggtcagcggcagcagg

SEQ ID NO: 35 - DNA, 21 bp, Artificial Sequence
  Signr1-RT-R
tcagcaggagcccagccaaga

SEQ ID NO: 36 - DNA, 20 bp, Artificial Sequence
  Signr3-RT-F
gggcccaactggtcatcata

SEQ ID NO: 37 - DNA, 20 bp, Artificial Sequence
  Signr3-RT-R
agcgtgtaaagctgggtgac

SEQ ID NO: 38 - DNA, 19 bp, Artificial Sequence
  rDNA-F
aggattagataccctggta

SEQ ID NO: 39 - DNA, 17 bp, Artificial Sequence
  rDNA-R
  misc_feature (2)..(3): R = A or G
crrcacgagctgacgac

SEQ ID NO: 40 - protein, 72 aa, Artificial Sequence
  Combination of hly signal sequence, DlaT 208- 222, DlaT 245-259 and DlaT 109-123
MAKKIMLVFITLILVSLPIAQQTEAKDVGSSVPAAPAAAPAAAAAPVAPPAPPAPAAVAPPAPVAPSAPVAA

SEQ ID NO: 41 - DNA, 219 bp, Artificial Sequence
  Combination of hly signal sequence, DlaT 208- 222, DlaT 245-259 and DlaT 109-123
atggccaaaaaaataatgctagtttttattacacttatattagttagtctaccaattgcgcaacaaactgaagcaaaggatgttggtagtagtgtcccagctgcaccagctgcagcaccagcagctgctgctgctccagtagcaccgcctgcgccgccagctccggcagctgtagcgccacctgcaccggtagcacctagtgcaccagttgctgcatag

SEQ ID NO: 42 - protein, 45 aa, Artificial Sequence
  Obtained from dlat
VGSSVPAAPAAAPAAVAPPAPVAPSAPVAAAAAPVAPPAPPAPAA

SEQ ID NO: 43 - protein, 45 aa, Artificial Sequence
  Obtained from dlat
VGSSVPAAPAAAPAAAAAPVAPPAPPAPAAVAPPAPVAPSAPVAA

SEQ ID NO: 44 - protein, 45 aa, Artificial Sequence
  Obtained from dlat
VAPPAPVAPSAPVAAVGSSVPAAPAAAPAAAAAPVAPPAPPAPAA

SEQ ID NO: 45 - protein, 45 aa, Artificial Sequence
  Obtained from dlat
VAPPAPVAPSAPVAAAAAPVAPPAPPAPAAVGSSVPAAPAAAPAA

SEQ ID NO: 46 - protein, 45 aa, Artificial Sequence
  Obtained from dlat
AAAPVAPPAPPAPAAVAPPAPVAPSAPVAAVGSSVPAAPAAAPAA

SEQ ID NO: 47 - protein, 45 aa, Artificial Sequence
  Obtained from dlat
AAAPVAPPAPPAPAAVGSSVPAAPAAAPAAVAPPAPVAPSAPVAA

SEQ ID NO: 48 - protein, 589 aa, Propionibacterium freudenreichii
MSTEVTLPELGESVTEATVSRWLKEVGDHVDADEPLLEVSTDKVDTEIPSPVAGTLLEIKFNEDDTAPVGAVLAVVGDPAEAPAGAPAPAAPAPAAAPVPPPPPIPAAAAAPVAPPAPPAPAAAAPAAAPAGQGTPVTLPELGESVTEATVSRWLKEVGDPVEADEPLLEVSTDKVDTEIPSPVAGTLTEIHVKEDETAEVGSVLGVVGSSVPAAPAAAPAAPAAPAAPVAPPAPAVPVAAPAPVAPPAPVAPSAPVAAPPAAPTPAAAVPVNPPAPRATEGAAFTGYVTPLVRKLAQQNDVDLNQVTGTGVGGRIRKQDVLDAAAAKQKAAAAPARPKAAPSPDAGKRGTTEKMSNLRKIVASRMTESLQTSAQLTATVEVDLSAVARVRAKAKDDFRKREGVGLTYLAFITQAATEALRQYPKVNASIDTEAGTITYHDSEDIGIAVDTPKGLMVPVIKGAGDLNVGGVAKSIGDLAARARDGKIGPDELSGATFTITNYGSTGTLFDTPIINLPNAAILGTGAMVKRPVVVSDEYGNDTIAIRPMMYLSMTYDHRLVDGADASRFLSFVKARLEEGDFGAEFGLNQ